(19) United States
(12) Patent Application Publication
(10) Pub. No.: US 2002/0107373 A1
(43) Pub. Date: Aug. 8, 2002

(54) 49937, 49931, and 49933, novel human transporter family members and uses thereof

(76) Inventors: Rory A.J. Curtis, Southborough, MA (US); Miyoung Chun, Belmont, MA (US)

Correspondence Address: LAHIVE & COCKFIELD, 28 STATE STREET, BOSTON, MA, 02109, US

(21) Appl. No.: 09/935,291

(22) Filed: Aug. 21, 2001

Related U.S. Application Data
(60) Provisional application No. 60/226,504, filed on Aug. 21, 2000.
(60) Provisional application No. 60/250,932, filed on Nov. 30, 2000.

(51) Int. Cl. C07H 21/02; C07H 21/04

(52) U.S. Cl. 536/23.1

(57) ABSTRACT

The invention provides isolated nucleic acid molecules, designated HEAT nucleic acid molecules, which encode novel transporter family members. The invention also provides antisense nucleic acid molecules, recombinant expression vectors containing HEAT nucleic acid molecules, host cells into which the expression vectors have been introduced, and nonhuman transgenic animals in which a HEAT gene has been introduced or disrupted. The invention still further provides isolated HEAT proteins, fusion proteins, antigenic peptides and anti-HEAT antibodies. Diagnostic and therapeutic methods utilizing compositions of the invention are also provided.

### RELATED APPLICATIONS

This application claims the benefit of U.S. Provisional Application No. 60/226,504, filed on Aug. 21, 2000, and U.S. Provisional Application No. 60/250,932, filed on Nov. 30, 2000, incorporated herein in their entirety by this reference.

### BACKGROUND OF THE INVENTION

The E1-E2 ATPase family is a large superfamily of cation transport enzymes that contains at least 80 members found in diverse organisms such as bacteria, archaea, and eukaryotes (Palmgren, M. G. and Axelsen, K. B. (1998) Biochim. Biophys. Acta. 1365:37-45). These enzymes are involved in ATP hydrolysis-dependent transmembrane movement of a variety of inorganic cations (e.g., H⁺, Na⁺, K⁺, Ca²⁺, Cu²⁺, Cd⁺, and Mg²⁺ ions) across a concentration gradient, whereby the enzyme converts the free energy of ATP hydrolysis into electrochemical ion gradients. E1-E2 ATPases are also known as “P-type” ATPases, referring to the existence of a covalent high-energy phosphoryl-enzyme intermediate in the chemical reaction pathway of these transporters. The superfamily contains four major groups: Ca²⁺ transporting ATPases; Na⁺/K⁺-and gastric H⁺/K⁺ transporting ATPases; plasma membrane H⁺ transporting ATPases of plants, fungi, and lower eukaryotes; and all bacterial P-type ATPases (Kuhlbrandt et al. (1998) Curr. Opin. Struct. Biol. 8:510-516).

E1-E2 ATPases are phosphorylated at a highly conserved DKTG sequence. Phosphorylation at this site is thought to control the enzyme's substrate affinity. Most E1-E2 ATPases contain ten alpha-helical transmembrane domains, although additional domains may be present. A majority of known gated-pore translocators contain twelve alpha-helices, including Na⁺/H⁺ antiporters (West (1997) Biochim. Biophys. Acta 1331:213-234).

Members of the E1-E2 ATPase superfamily are able to generate electrochemical ion gradients which enable a variety of processes in the cell such as absorption, secretion, transmembrane signaling, nerve impulse transmission, excitation/contraction coupling, and growth and differentiation (Scarborough (1999) Curr. Opin. Cell Biol. 11:517-522). These molecules are thus critical to normal cell function and well-being of the organism.

### SUMMARY OF THE INVENTION

The present invention is based, at least in part, on the discovery of novel calcium transporter family members, referred to interchangeably herein as “P-type ATPase”, “E1-E2 ATPase”, “human E1-E2 ATPase”, or “HEAT” nucleic acid and protein molecules (e.g., HEAT-1, HEAT-2 and HEAT-3). The HEAT nucleic acid and protein molecules of the present invention are useful as modulating agents in regulating a variety of cellular processes, e.g., tone regulation in vascular smooth muscle cells, cellular growth and/or proliferation, and/or angiogenesis. Accordingly, in one aspect, this invention provides isolated nucleic acid molecules encoding HEAT proteins or biologically active portions thereof, as well as nucleic acid fragments suitable as primers or hybridization probes for the detection of HEAT-encoding nucleic acids.

In one embodiment, the invention features an isolated nucleic acid molecule that includes the nucleotide sequence set forth in SEQ ID NO:1, SEQ ID NO:3, SEQ ID NO:5, SEQ ID NO:7, SEQ ID NO:8, or SEQ ID NO:10. In another embodiment, the invention features an isolated nucleic acid molecule that encodes a polypeptide including the amino acid sequence set forth in SEQ ID NO:2, 6, or 9. In another embodiment, the invention features an isolated nucleic acid molecule that includes the nucleotide sequence contained in the plasmid deposited with ATCC® as Accession Number ______, ______, or ______.

In still other embodiments, the invention features isolated nucleic acid molecules including nucleotide sequences that are substantially identical (e.g., 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical) to the nucleotide sequence set forth as SEQ ID NO:1, SEQ ID NO:3, SEQ ID NO:5, SEQ ID NO:7, SEQ ID NO:8, OR SEQ ID NO:10. The invention further features isolated nucleic acid molecules including at least 30 contiguous nucleotides of the nucleotide sequence set forth as SEQ ID NO:1, SEQ ID NO:3, SEQ ID NO:5, SEQ ID NO:7, SEQ ID NO:8, OR SEQ ID NO:10. In another embodiment, the invention features isolated nucleic acid molecules which encode a polypeptide including an amino acid sequence that is substantially identical (e.g., 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical) to the amino acid sequence set forth as SEQ ID NO:2, 6, or 9. Also featured are nucleic acid molecules which encode allelic variants of the polypeptide having the amino acid sequence set forth as SEQ ID NO:2, 6, or 9. In addition to isolated nucleic acid molecules encoding full-length polypeptides, the present invention also features nucleic acid molecules which encode fragments, for example, biologically active or antigenic fragments, of the full-length polypeptides of the present invention (e.g., fragments including at least 10 contiguous amino acid residues of the amino acid sequence of SEQ ID NO:2, 6, or 9). In still other embodiments, the invention features nucleic acid molecules that are complementary to, antisense to, or hybridize under stringent conditions to the isolated nucleic acid molecules described herein.

In a related aspect, the invention provides vectors including the isolated nucleic acid molecules described herein (e.g., HEAT-encoding nucleic acid molecules). Such vectors can optionally include nucleotide sequences encoding heterologous polypeptides. Also featured are host cells including such vectors (e.g., host cells including vectors suitable for producing HEAT nucleic acid molecules and polypeptides).

In another aspect, the invention features isolated HEAT polypeptides and/or biologically active or antigenic fragments thereof. Exemplary embodiments feature a polypeptide including the amino acid sequence set forth as SEQ ID NO:2, 6, or 9, a polypeptide including an amino acid sequence at least 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the amino acid sequence set forth as SEQ ID NO:2, 6, or 9, a polypeptide encoded by a nucleic acid molecule including a nucleotide sequence at least 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the nucleotide sequence set forth as SEQ ID NO:1, SEQ ID NO:3, SEQ ID NO:5, SEQ ID NO:7, SEQ ID NO:8, OR SEQ ID NO:10. Also featured are fragments of the full-length polypeptides described herein (e.g., fragments including at least 10 contiguous amino acid residues of the sequence set forth as SEQ ID NO:2, 6, or 9) as well as allelic variants of the polypeptide having the amino acid sequence set forth as SEQ ID NO:2, 6,or 9.

The HEAT polypeptides and/or biologically active or antigenic fragments thereof, are useful, for example, as reagents or targets in assays applicable to treatment and/or diagnosis of cardiovascular disorders. In one embodiment, a HEAT polypeptide or fragment thereof has a HEAT activity. In another embodiment, a HEAT polypeptide or fragment thereof has at least one or more of the following domains or motifs: a transmembrane domain, an E1-E2 ATPase domain, an E1-E2 ATPases phosphorylation site, an N-terminal large extramembrane domain, a C-terminal large extramembrane domain, a P-type ATPase sequence 1 motif, a P-type ATPase sequence 2 motif, and/or a P-type ATPase sequence 3 motif and, optionally, has a HEAT activity. In a related aspect, the invention features antibodies (e.g., antibodies which specifically bind to any one of the polypeptides, as described herein) as well as fusion polypeptides including all or a fragment of a polypeptide described herein.

The present invention further features methods for detecting HEAT polypeptides and/or HEAT nucleic acid molecules, such methods featuring, for example, a probe, primer or antibody described herein. Also featured are kits for the detection of HEAT polypeptides and/or HEAT nucleic acid molecules. In a related aspect, the invention features methods for identifying compounds which bind to and/or modulate the activity of a HEAT polypeptide or HEAT nucleic acid molecule described herein. Also featured are methods for modulating a HEAT activity.

In other embodiments, the invention provides methods for identifying a subject having a cardiovascular disorder, or at risk for developing a cardiovascular disorder; methods for identifying a compound capable of treating a cardiovascular disorder characterized by aberrant HEAT nucleic acid expression or HEAT polypeptide activity; and methods for treating a subject having a cardiovascular disorder characterized by aberrant HEAT polypeptide activity or aberrant HEAT nucleic acid expression.

Other features and advantages of the invention will be apparent from the following detailed description and claims.

### BRIEF DESCRIPTION OF THE DRAWINGS

FIGS. 1A-1D depict the nucleotide sequence of the human HEAT-1 cDNA and the corresponding amino acid sequence. The nucleotide sequence corresponds to nucleic acids 1 to 4055 of SEQ ID NO:1. The amino acid sequence corresponds to amino acids 1 to 1180 of SEQ ID NO:2. The coding region without the 5′ and 3′ untranslated regions of the human HEAT-1 gene is shown in SEQ ID NO:3.

FIG. 2 depicts a structural, hydrophobicity, and antigenicity analysis of the human HEAT-1 polypeptide. The locations of the 12 transmembrane domains, as well as the E1-E2 ATPase domain, are indicated.

FIG. 3 depicts the results of a search in the HMM database in PFAM which resulted in the identification of an “E1-E2 ATPase domain” in the human HEAT-1 polypeptide (SEQ ID NO:2).

FIGS. 4A-4B depict a Clustal W (1.74) multiple sequence alignment of the human HEAT-1 amino acid sequence (Fbh49937; SEQ ID NO:2) and the amino acid sequence of a C. elegans l cation-transporting ATPase (YH2Melegans; SEQ ID NO:4; GenBank Accession No. Q27533). Amino acid identities are indicated by stars.

FIG. 5 depicts the expression levels of human HEAT-1 mRNA in various human cell types and tissues, as determined by Taqman analysis. Column: (1) normal artery; (2) normal vein; (3) aortic smooth muscle cells (early); (4) coronary smooth muscle cells; (5) umbilical vein endothelial cells (static); (6) umbilical vein endothelial cells (shear); (7) normal heart; (8) heart (congestive heart failure); (9) kidney; (10) skeletal muscle; (11) normal adipose tissue; (12) pancreas; (13) primary osteoblasts; (14) differentiated osteoclasts; (15) normal skin; (16) normal spinal cord; (17) normal brain cortex; (18) normal brain hypothalamus; (19) nerve; (20) dorsal root ganglion; (21) glial cells (astrocytes); (22) glioblastoma; (23) normal breast; (24) breast tumor; (25) normal ovary; (26) ovarian tumor; (27) normal prostate; (28) prostate tumor; (29) epithelial cells (prostate); (30) normal colon; (31) colon tumor; (32) normal lung; (33) lung tumor; (34) lung (chronic obstructive pulmonary disease); (35) colon (inflammatory bowel disease); (36) normal liver; (37) liver fibrosis; (38) dermal cells (fibroblasts); (39) normal spleen; (40) normal tonsil; (41) lymph node; (42) small intestine; (43) skin (decubitus); (44) synovium; (45) bone marrow mononuclear cells; (46) activated peripheral blood mononuclear cells.

FIG. 6 depicts the expression levels of human HEAT-1 mRNA in various vascular rich organs, as determined by Taqman analysis. Column: (1) confluent microvascular endothelial cells; (2) aortic smooth muscle cells; (3) fetal heart; (4) normal heart atrium; (5) normal heart atrium; (6) normal heart ventricle; (7) normal heart ventricle; (8) normal heart ventricle; (9) normal heart ventricle; (10) normal heart ventricle; (11) diseased heart ventricle; (12) diseased heart ventricle; (13) diseased heart ventricle; (14) normal kidney; (15) normal kidney; (16) normal kidney; (17) normal kidney; (18) normal kidney; (19) hypertensive kidney; (20) hypertensive kidney; (21) hypertensive kidney; (22) hypertensive kidney; (23) skeletal muscle; (24) skeletal muscle; (25) liver; (26) liver; (27) normal fetal adrenal gland; (28) Wilms tumor; (29) Wilms tumor; (30) normal spinal cord; (31) diseased cartilage.

FIG. 7 depicts the expression levels of human HEAT-1 mRNA in various human and monkey vessels, as determined by Taqman analysis. Column: (1) human aortic smooth muscle cells; (2) human microvascular endothelial cells; (3) human adipose tissue; (4) human normal carotid artery; (5) human normal carotid artery; (6) human normal muscular artery; (7) human diseased iliac artery; (8) human diseased tibial artery; (9) human diseased aorta; (10) human normal saphenous vein; (11) human normal saphenous vein; (12) human normal saphenous vein; (13) human normal saphenous vein; (14) human diseased saphenous vein; (15) human normal vein; (16) human normal vein; (17) human normal vein; (18) monkey normal coronary artery; (19) monkey normal coronary artery; (20) monkey normal coronary artery; (21) monkey normal coronary artery; (22) monkey normal vein; (23) no transcriptase control.

FIG. 8 depicts the expression levels of human HEAT-1 mRNA in various human coronary vascular cell types, as well as other cell types, as determined by Taqman analysis. Column: (1) aortic smooth muscle cells; (2) aortic smooth muscle cells; (3) aortic smooth muscle cells; (4) coronary smooth muscle cells; (5) coronary smooth muscle cells; (6) coronary smooth muscle cells; (7) coronary smooth muscle cells; (8) macrophages; (9) macrophages treated with IFNγ; (10) macrophages treated with CD40; (11) macrophages treated with LPS; (12) umbilical vein endothelial cells; (13) microvascular endothelial cells; (14) aortic endothelial cells; (15) aortic endothelial cells; (16) cortex renal epithelium; (17) renal proximal tubule epithelium; (18) mesangial cells; (19) skeletal muscle; (20) skeletal muscle; (21) lung fibroblasts.

FIG. 9 depicts the expression levels of human HEAT-1 mRNA in various human endothelial cell paradigms, as determined by Taqman analysis. Column: (1) umbilical vein endothelial cells (static); (2) umbilical vein endothelial cells (laminar shear stress); (3) umbilical vein endothelial cells (static); (4) umbilical vein endothelial cells (laminar shear stress); (5) umbilical vein endothelial cells (proliferating); (6) umbilical vein endothelial cells (confluent); (7) umbilical vein endothelial cells (without growth factor treatment); (8) umbilical vein endothelial cells (treated with IL-1); (9) cardiac microvascular endothelial cells (proliferating); (10) cardiac microvascular endothelial cells (confluent); (11) cardiac microvascular endothelial cells (proliferating); (12) cardiac microvascular endothelial cells (confluent); (13) lung microvascular endothelial cells (proliferating); (14) lung microvascular endothelial cells (confluent); (15) lung microvascular endothelial cells (without growth factor treatment); (16) lung microvascular endothelial cells (proliferating); (17) lung microvascular endothelial cells (confluent); (18) aortic cells (control 4 h); (19) aortic cells (TNF treated 4 h); (20) aortic cells (control 14 h); (21) aortic cells (TNF treated 14 h); (22) 293 cells; (23) lung microvascular endothelial cells (Matrigel 5 h); (24) lung microvascular endothelial cells (Matrigel 25 h); (25) lung microvascular endothelial cells (proliferating); (26) lung microvascular endothelial cells (without growth factor treatment).

FIG. 10 depicts the expression levels of human HEAT-1 mRNA in cells subjected to various laminar shear stress treatments, as determined by Taqman analysis. Column: (1) static (control); (2) laminar shear stress (LSS); (3) LSS+1 h up; (4) LSS+1 h down; (5) static (control); (6) LSS; (7) LSS+6 h up; (8) static (control); (9) LSS; (10) LSS+6 h down.

FIGS. 11A-11E depict the nucleotide sequence of the human HEAT-2 cDNA and the corresponding amino acid sequence. The nucleotide sequence corresponds to nucleic acids 1 to 7249 of SEQ ID NO:5. The amino acid sequence corresponds to amino acids 1 to 1256 of SEQ ID NO:6. The coding region without the 5′ or 3′ untranslated regions of the human HEAT-2 gene is shown in SEQ ID NO:7.

FIG. 12 depicts the results of a search in the HMM database which resulted in the identification of an “E1-E2 ATPase domain” in the human HEAT-2 polypeptide (SEQ ID NO:6).

FIG. 13 depicts a structural, hydrophobicity, and antigenicity analysis of human HEAT-2. The locations of the 12 transmembrane domains, as well as the E1-E2 ATPase domain, are indicated.

FIGS. 14A-14B depict a Clustal W (1.74) multiple sequence alignment of the human HEAT-2 amino acid sequence (Fbh49931IFL; SEQ ID NO:6) and the amino acid sequence of a C. elegans cation-transporting ATPase (YH2Melegans; SEQ ID NO:4; GenBank Accession No. Q27533). Amino acid identities are indicated by stars. The twelve transmembrane domains, as well as the phosphorylation site, are indicated by boxes.

FIG. 15 depicts the expression levels of human HEAT-2 mRNA in various human cell types and tissues, as determined by Taqman analysis. Column: (1) normal aorta; (2) normal fetal heart; (3) normal heart; (4) heart (congestive heart failure); (5) normal vein; (6) aortic smooth muscle cells; (7) normal spinal cord; (8) brain (normal cortex); (9) brain (hypothalamus); (10) glial cells (astrocytes); (11) brain (glioblastoma); (12) normal breast; (13) breast tunor (infiltrating ductal carcinoma); (14) normal ovary; (15) ovarian tumor; (16) pancreas; (17) normal prostate; (18) prostate tumor; (19) normal colon; (20) colon tumor; (21) colon (inflammatory bowel disease); (22) normal kidney; (23) normal liver; (24) fibrotic liver; (25) normal fetal liver; (26) normal lung; (27) lung tumor; (28) lung (chronic obstructive pulmonary disease); (29) normal spleen; (30) normal tonsil; (31) normal lymph node; (32) normal thymus; (33) epithelial cells (from prostate); (34) aortic endothelial cells; (35) skeletal muscle; (36) dermal fibroblasts; (37) normal skin; (38) normal adipose tissue; (39) primary osteoblasts; (40) undifferentiated osteoblasts; (41) differentiated osteoblasts; (42) osteoclasts; (43) aortic smooth muscle cells (early); (44) aortic smooth muscle cells (late); (45) human umbilical vein endothelial cells (shear); (46) human umbilical vein endothelial cells (static).

FIG. 16 depicts the expression levels of human HEAT-2 mRNA in various vascular rich organs, as determined by Taqman analysis. Column: (1) normal human heart; (2) normal human heart; (3) normal human heart; (4) normal human heart; (5) normal human heart; (6) normal human heart; (7) normal human heart; (8) normal human heart; (9) diseased human heart; (10) diseased human right ventricle; (11) diseased human left ventricle; (12) normal monkey heart; (13) normal monkey heart; (14) normal monkey heart; (15) normal human kidney; (16) normal human kidney; (17) normal human kidney; (18) normal human kidney; (19) normal human kidney; (20) human hypertensive kidney; (21) human hypertensive kidney; (22) human hypertensive kidney; (23) human hypertensive kidney; (24) human hypertensive kidney; (25) human liver; (26) human liver; (27) human liver; (28) human skeletal muscle; (29) human skeletal muscle; (30) human skeletal muscle.

FIG. 17 depicts the expression levels of human HEAT-2 mRNA in various human and monkey vessels, as determined by Taqman analysis. Column: (1) human adipose tissue; (2) human normal artery; (3) human normal artery; (4) human carotid artery; (5) human carotid artery; (6) human normal artery; (7) human diseased artery; (8) human diseased artery; (9) human diseased artery; (10) human normal vein; (11) human normal vein; (12) human vein; (13) human vein; (14) human normal vein; (15) human varicose vein; (16) confluent human microvascular endothelial cells; (17) human aortic smooth muscle cells; (18) monkey aorta; (19) monkey aorta; (20) monkey aorta; (21) monkey artery; (22) monkey artery; (23) monkey renal artery; (24) monkey renal artery; (25) monkey renal artery; (26) monkey renal artery; (27) monkey renal artery; (28) monkey coronary artery; (29) monkey coronary artery; (30) monkey coronary artery; (31) monkey coronary artery; (32) monkey coronary artery; (33) monkey coronary artery; (34) monkey coronary artery.

FIG. 18 depicts the expression levels of human HEAT-2 mRNA in various cell types and tissues, as determined by transcriptional profiling analysis. Column: (1) human aortic smooth muscle cells; (2) human coronary artery smooth muscle cells; (3) human umbilical vein endothelial cells; (4) human microvascular endothelial cells (lung); (5) monkey aorta; (6) monkey vein; (7) monkey heart; (8) monkey liver.

FIG. 19 depicts the expression levels of human HEAT-2 mRNA in various human coronary vascular cell types, as well as other cell types, as determined by Taqman analysis. Column: (1) aortic smooth muscle cells; (2) aortic smooth muscle cells; (3) aortic smooth muscle cells; (4) aortic smooth muscle cells; (5) coronary smooth muscle cells; (6) coronary smooth muscle cells; (7) coronary smooth muscle cells; (8) coronary smooth muscle cells; (9) macrophages; (10) macrophages treated with IFNγ; (11) macrophages treated with CD40; (12) macrophages treated with LPS; (13) umbilical vein endothelial cells; (14) microvascular endothelial cells; (15) aortic endothelial cells; (16) coronary artery endothelial cells; (17) coronary artery endothelial cells; (18) cortex renal epithelium; (19) renal proximal tubule epithelium; (20) mesangial cells; (21) skeletal muscle; (22) skeletal muscle; (23) lung fibroblasts.

FIG. 20 depicts the expression levels of human HEAT-2 mRNA in various human endothelial cell paradigms of shear stress, as determined by Taqman analysis. Column: (1) umbilical vein endothelial cells (static); (2) umbilical vein endothelial cells (shear regulated); (3) umbilical vein endothelial cells (proliferating); (4) umbilical vein endothelial cells (confluent); (5) umbilical vein endothelial cells (without growth factor treatment); (6) umbilical vein endothelial cells (Interleukin-1 stimulated); (7) microvascular endothelial cells (proliferating); (8) microvascular endothelial cells (confluent); (9) microvascular endothelial cells (proliferating); (10) microvascular endothelial cells (confluent); (11) microvascular endothelial cells (proliferating); (12) microvascular endothelial cells (confluent); (13) microvascular endothelial cells (without growth factor treatment); (14) coronary microvascular endothelial cells (proliferating); (15) coronary microvascular endothelial cells (confluent); (16) microvascular endothelial cells (5% serum plus growth factors); (17) microvascular endothelial cells (5% serum without growth factors); (18) microvascular endothelial cells (hEGF treated); (19) microvascular endothelial cells (VEGF treated); (20) microvascular endothelial cells (bFGF treated); (21) microvascular endothelial cells (IGF treated); (22) 293 cells; (23) umbilical vein endothelial cells (static 25 h); (24) umbilical vein endothelial cells (laminar shear stress); (25) umbilical vein endothelial cells (laminar shear stress+1 h up); (26) umbilical vein endothelial cells (laminar shear stress+1 h down); (27) umbilical vein endothelial cells (static 30 h); (28) umbilical vein endothelial cells (laminar shear stress); (29) umbilical vein endothelial cells (laminar shear stress +6 h up); (30) umbilical vein endothelial cells (static 30 h); (31) umbilical vein endothelial cells (laminar shear stress); (32) umbilical vein endothelial cells (laminar shear stress+6 h down).

FIG. 21 depicts the expression level of human HEAT-2 mRNA human microvascular endothelial cells under conditions of tube formation (growth on Matrigel). Column: (1) Matrigel (5 h); (2) Matrigel (25 h); (3) proliferating; (4) confluent.

FIGS. 22A-22D depict the nucleotide sequence of the human HEAT-3 cDNA and the corresponding amino acid sequence. The nucleotide sequence corresponds to nucleic acids 1 to 3919 of SEQ ID NO:8. The amino acid sequence corresponds to amino acids 1 to 1204 of SEQ ID NO:9. The coding region without the 5′ or 3′ untranslated regions of the human HEAT-3 gene is shown in SEQ ID NO:10.

FIG. 23 depicts the results of a search in the HMM database which resulted in the identification of an “E1-E2 ATPase domain” in the human HEAT-3 polypeptide (SEQ ID NO:9).

FIG. 24 depicts a structural, hydrophobicity, and antigenicity analysis of human HEAT-3. The locations of the 12 transmembrane domains, as well as the E1-E2 ATPase domain, are indicated.

FIGS. 25A-25B depict a Clustal W (1.74) multiple sequence alignment of the human HEAT-3 amino acid sequence (Fbh49933FL1; SEQ ID NO:8) and the amino acid sequence of a C. elegans cation-transporting ATPase (YE56elegans; SEQ ID NO:12; GenBank Accession No. P90747). Amino acid identities are indicated by stars. The twelve transmembrane domains, as well as the phosphorylation site, are indicated by boxes.

FIG. 26 depicts the expression levels of human HEAT-3 mRNA in various human cell types and tissues, as determined by Taqman analysis. Column: (1) normal aorta; (2) normal fetal heart; (3) normal heart; (4) heart (congestive heart failure); (5) normal vein; (6) normal spinal cord; (7) normal brain cortex; (8) normal brain hypothalamus; (9) glial cells (astrocytes); (10) glioblastoma (brain); (11) normal breast; (12) breast tumor (infiltrating ductal carcinoma); (13) normal ovary; (14) ovarian tumor; (15) pancreas; (16) normal prostate; (17) prostate tumor; (18) normal colon; (19) colon tumor; (20) colon (inflammatory bowel disease); (21) normal kidney; (22) normal liver; (23) liver fibrosis; (24) normal fetal liver; (25) normal lung; (26) lung tumor; (27) lung (chronic obstructive pulmonary disease); (28) normal spleen; (29) normal tonsil; (30) normal lymph node; (31) normal thymus; (32) epithelial cells (prostate); (33) endothelial cells (aortic); (34) normal skeletal muscle; (35) fibroblasts (dermal); (36) normal skin; (37) normal adipose tissue; (38) primary osteoblasts; (39) undifferentiated osteoblasts; (40) differentiated osteoblasts; (41) osteoclasts; (42) aortic smooth muscle cells (early); (43) aortic smooth muscle cells (late); (44) umbilical vein endothelial cells (laminar shear stress); (45) umbilical vein endothelial cells (static); (46) undifferentiated osteoclasts.

FIG. 27 depicts the expression levels of human HEAT-3 mRNA in various vascular rich organs, as determined by Taqman analysis. Column: (1) normal heart; (2) normal heart; (3) normal heart; (4) normal heart; (5) normal heart; (6) normal heart; (7) normal heart; (8) normal heart; (9) diseased heart; (10) diseased right ventricle; (11) normal fetal heart; (12) normal kidney; (13) normal kidney; (14) normal kidney; (15) normal kidney; (16) normal kidney; (17) hypertensive kidney; (18) hypertensive kidney; (19) hypertensive kidney; (20) hypertensive kidney; (21) hypertensive kidney; (22) skeletal muscle; (23) skeletal muscle; (24) skeletal muscle; (25) liver; (26) liver; (27) normal monkey heart; (28) normal monkey heart; (29) normal monkey heart; (30) normal monkey heart; (31) smooth muscle cells (SMC); (32) confluent human microvascular endothelial cells (HMVECs); (33) M human umbilical vein endothelial cells (HUVECs); (34) human umbilical vein endothelial cells (HUVECs)-vehicle; (35) M human amniotic endothelial cells (HAECs); (36) human amniotic endothelial cells (HAECs)-vehicle.

FIG. 28 depicts the expression levels of human HEAT-3 mRNA in various human vessels, as determined by Taqman analysis. Column: (1) aortic smooth muscle cells; (2) microvascular endothelial cells; (3) adipose tissue; (4) normal artery; (5) normal artery; (6) normal artery; (7) diseased artery; (8) diseased artery; (9) diseased aorta; (10) normal vein; (11) normal vein; (12) normal vein; (13) normal vein; (14) diseased vein; (15) normal vein; (16) normal vein; (17) normal vein.

FIG. 29 depicts the expression levels of human HEAT-3 mRNA in various human coronary vascular cell types, as well as other cell types, as determined by Taqman analysis. Column: (1) aortic smooth muscle cells; (2) aortic smooth muscle cells; (3) coronary smooth muscle cells; (4) coronary smooth muscle cells; (5) coronary smooth muscle cells; (6) coronary smooth muscle cells; (7) macrophages; (8) macrophages treated with IFNγ; (9) macrophages treated with CD40; (10) macrophages treated with LPS; (11) microvascular endothelial cells; (12) aortic endothelial cells; (13) coronary artery endothelial cells; (14) cortex renal epithelium; (15) renal proximal tubule epithelium; (16) mesangial cells; (17) skeletal muscle.

FIG. 30 depicts an alignment of a region important in calcium binding from HEAT-1, HEAT-2, HEAT-3 with similar sequences from a number of E1-E2 ATPases of various substrate specificities from a number of different organisms. This region includes the sixth transmembrane domain from each of HEAT-1, HEAT-2, and HEAT-3, as well as a number of amino acid residues adjacent to the sixth transmembrane domain. Amino acid residues determined to be important for calcium binding by mutagenesis of a SERCA calcium-transporting E1-E2 ATPase are indicated (“SERCA mutagenesis”). Amino acid residues in this region that are critical for calcium binding are indicated in bold. Substrate specificities are as follows: Type V (calcium), Ca²⁺ (calcium), Cu²⁺ (copper), Na⁺/K⁺ (sodium/potassium), and PL (phospholipid).

FIG. 31 depicts the expression levels of human HEAT-3 mRNA in various human vessels, as determined by Taqman analysis. Column: (1) LC smooth muscle cells; (2) LC smooth muscle cells; (3) aortic smooth muscle cells; (4) human microvascular endothelial cells; (5) normal human carotid artery; (6) normal human carotid artery; (7) normal human muscular artery; (8) human diseased iliac artery; (9) human diseased tibial artery; (10) human diseased aorta; (11) human normal saphenous vein; (12) human normal saphenous vein; (13) human normal saphenous vein; (14) human normal saphenous vein; (15) human diseased saphenous vein; (16) human normal vein; (17) human normal saphenous vein.

### DETAILED DESCRIPTION OF THE INVENTION

The present invention is based, at least in part, on the discovery of novel calcium transporter family members, referred to interchangeably herein as “P-type ATPase”, “E1-E2 ATPase”, “human E1-E2 ATPase”, or “HEAT” nucleic acid and protein molecules (e.g., HEAT-1, HEAT-2 and HEAT-3). These novel molecules are members of the E1-E2 ATPase superfamily and are highly expressed in human vessels, endothelial cells, and vascular smooth muscle cells, e.g., coronary vascular smooth muscle cells.

The E1-E2 ATPases are involved in ATP hydrolysis-dependent transmembrane movement of inorganic cations (e.g., Ca²⁺ ions) across a concentration gradient. E1-E2 ATPases are phosphorylated at a highly conserved DKTG sequence. Phosphorylation at this site is thought to control the enzyme's substrate affinity. Most E1-E2 ATPases contain ten alpha-helical transmembrane domains, although additional domains may be present. Members of the E1-E2 ATPase superfamily are able to generate electrochemical ion gradients which enable a variety of processes in the cell such as absorption, secretion, transmembrane signaling, nerve impulse transmission, excitation/contraction coupling, and growth and differentiation.

As indicated in the Examples presented herein, the HEAT molecules of the present invention, e.g, HEAT-2, are up-regulated during shear, proliferation, and tube formation of endothelial cells and, thus, are believed to be involved in angiogenesis. Calcium ions are involved in the regulation of many cellular activities. In vascular smooth muscle cells, transient increases in intracellular calcium levels mediate contraction. Thus, maintenance of a low steady-state level of calcium is critical to maintaining proper cell function. Additionally, since the main determinant of the contraction-relaxation cycle of smooth muscle is calcium, calcium concentration is an important factor in the regulation of vascular tone. The normal concentration of calcium in the cell is in the submicromolar range, while the concentration in the extracellular compartment is in the millimolar range. In order to maintain intracellular calcium concentration in the submicromolar range, several mechanisms are operative in most cells. In smooth muscle cells, these regulatory mechanisms include calcium extrusion via Ca²⁺-transporting E1-E2 ATPases at the plasma membrane and at the sarcoplasmic/endoplasmic reticulum.

Thus, as the HEAT molecules of the present invention are Ca²⁺-transporting E1-E2 ATPases, and are highly expressed in vessels, endothelial cells, and vascular smooth muscle cells, these molecules are believed to be involved in vasotone regulation of vascular smooth muscle cells, e.g., coronary vascular smooth muscle cells. For example, activation of a HEAT molecule of the invention, e.g., HEAT-3, may result in decreased cytosolic calcium concentrations, thus reducing vascular tone. Inhibition of a HEAT molecule of the invention, e.g., HEAT-3, may result in decreased intracellular calcium store, which may subsequently lower the calcium release by vasopressor stimulation, thereby reducing vascular smooth muscle tone.

Accordingly, the HEAT molecules of the present invention provide novel diagnostic targets and therapeutic agents for cardivascular disorders. As used herein, the term “cardiovascular disorder” includes a disorder, disease or condition which affects the cardiovascular system, e.g., the heart or blood vessels. Cardiovascular disorders can detrimentally affect cellular functions such as calcium transport and inter- or intra-cellular communication; and tissue functions such as angiogenesis, vascular smooth muscle tone, vascular function, and cardiac function. Examples of cardiovascular disorders include cardiovascular disorders include hypertension, arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, atrial fibrillation, atrial flutter, dilated cardiomyopathy, idiopathic cardiomyopathy, myocardial infarction, coronary artery disease, coronary artery spasm, arrhythmia, atherosclerosis, transplant atherosclerosis, varicose veins, migraine headaches, cluster headaches, vascular disease, diabetic vascular disease, pulmonary vascular disease, peripheral vascular disease, renovascular hypertension, intravascular tumor, pulmonary vasculitis, vascular tone disorders in pregnancy, pulmonary capillaritis, peripheral arterial disease, idiopathic hypereosiniphilic syndrome, aortic aneurysm, respiratory disease, vasospasm, systemic sclerosis, preeclampsia, graft vessel disease, cardiac allograft vasculopathy, vascular ischemic injury, familial amyloidotic polyneuropathy, acute atherosis, cardiovascular disease, Kawasaki disease, ischemic syndromes, chronic heart failure, and fibrosis.

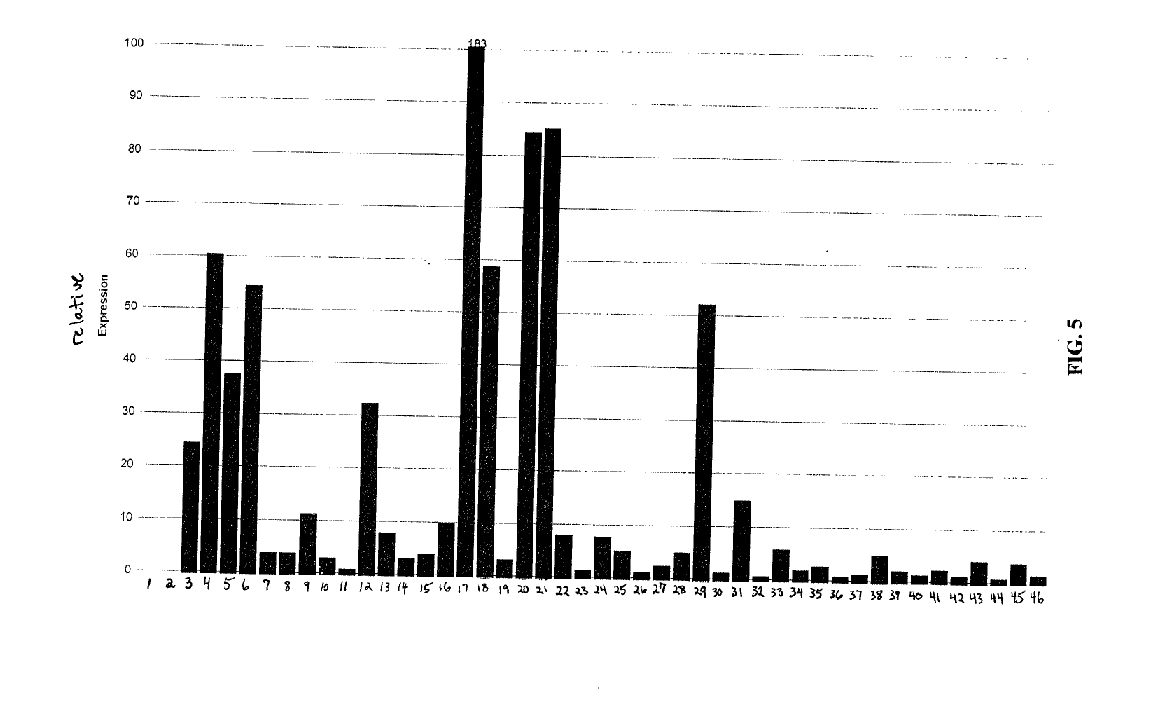

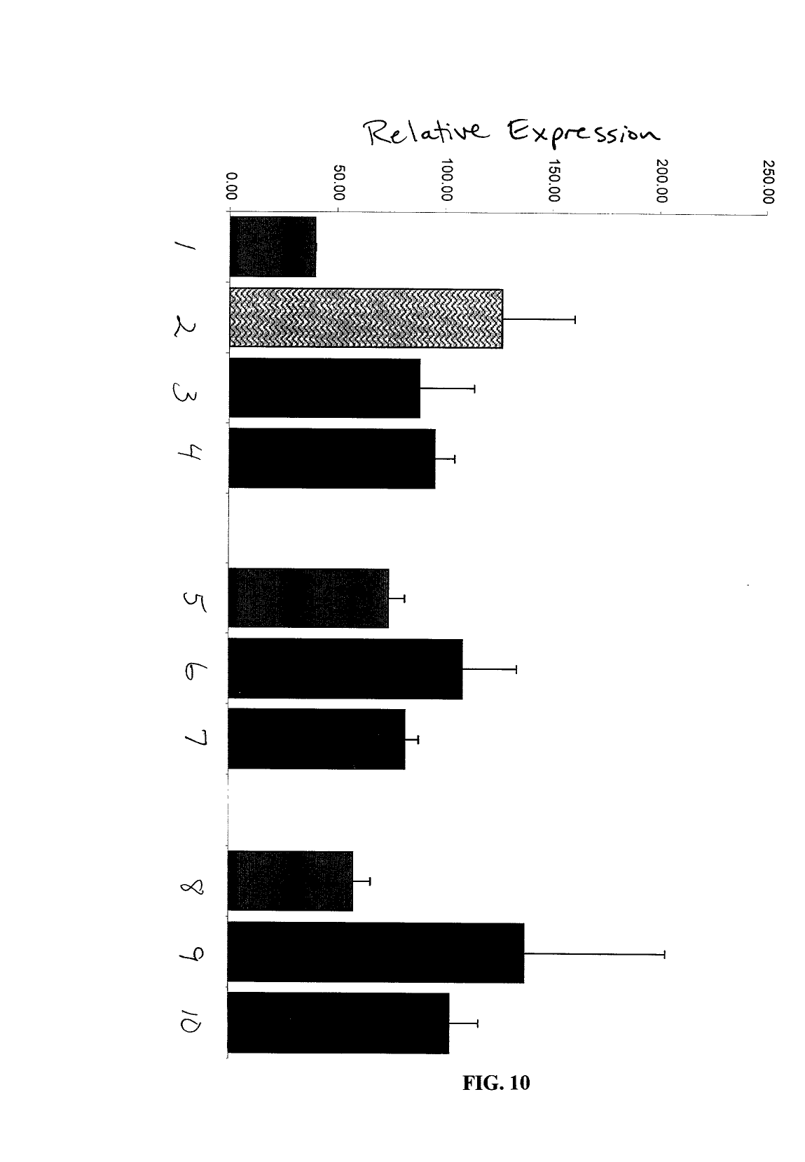

The HEAT molecules of the present invention further provide novel diagnostic targets and therapeutic agents for cellular proliferation, growth, or differentiation disorders. Cellular proliferation, growth, or differentiation disorders include those disorders that affect cell proliferation, growth, or differentiation processes. As used herein, a “cellular proliferation, growth, or differentiation process” is a process by which a cell increases in number, size or content, or by which a cell develops a specialized set of characteristics which differ from that of other cells. The HEAT molecules of the present invention are upregulated in various endothelial cell paradigms of shear, proliferation, and tube formation (see FIGS. 5, 9, 10, 15, 20, and 21), indicating that the HEAT molecules of the present invention are involved in cellular growth and proliferation. Thus, the HEAT molecules of the present invention may play a role in disorders characterized by aberrantly regulated growth, proliferation, or differentiation. Such disorders include cancer, e.g., carcinoma, sarcoma, or leukemia; tumor angiogenesis and metastasis; skeletal dysplasia; hepatic disorders; myelodysplastic syndromes; and hematopoietic and/or myeloproliferative disorders.

Other disorders related to angiogenesis and which may, therefore, be treated using the molecules described herein, include diabetic retinopathy, neovascularization (e.g., intraocular neovascularization), psoriasis, endometriosis, Grave's disease, ischemic disease, chronic inflammatory diseases, macular degeneration, neovascular glaucoma, retinal fibroplasia, uveitis, eye diseases associated with choroidal neovascularization and iris neovascularization, hereditary hemorrhagic telangiectasia, fibrodysplasia ossificans progressiva, idiopathic pulmonary fibrosis, autosomal dominant polycystic kidney disease, synovitis, familial exudative vitreoretinopathy (FEVR), Alagille syndrome, Knobloch syndrome, disseminated lymphangiomatosis, toxic epidermal necrolysis, Von Hippel Lindau disease (VHL), microbial-related dysplastic and neoplastic angiomatous proliferative processes (e.g., verruga peruana (VP)), Proteus syndrome (PS), Castleman's disease, and Klippel-Trenaunay-Weber syndrome.

Additional disorders that may be treated using the molecules of the present invention include disorders affecting tissues in which HEAT protein is expressed (e.g., vessels, endothelial cells, and vascular smooth muscle cells).

The term “family” when referring to the protein and nucleic acid molecules of the present invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin or alternatively, can contain homologues of non-human origin, e.g., rat or mouse proteins. Members of a family can also have common functional characteristics.

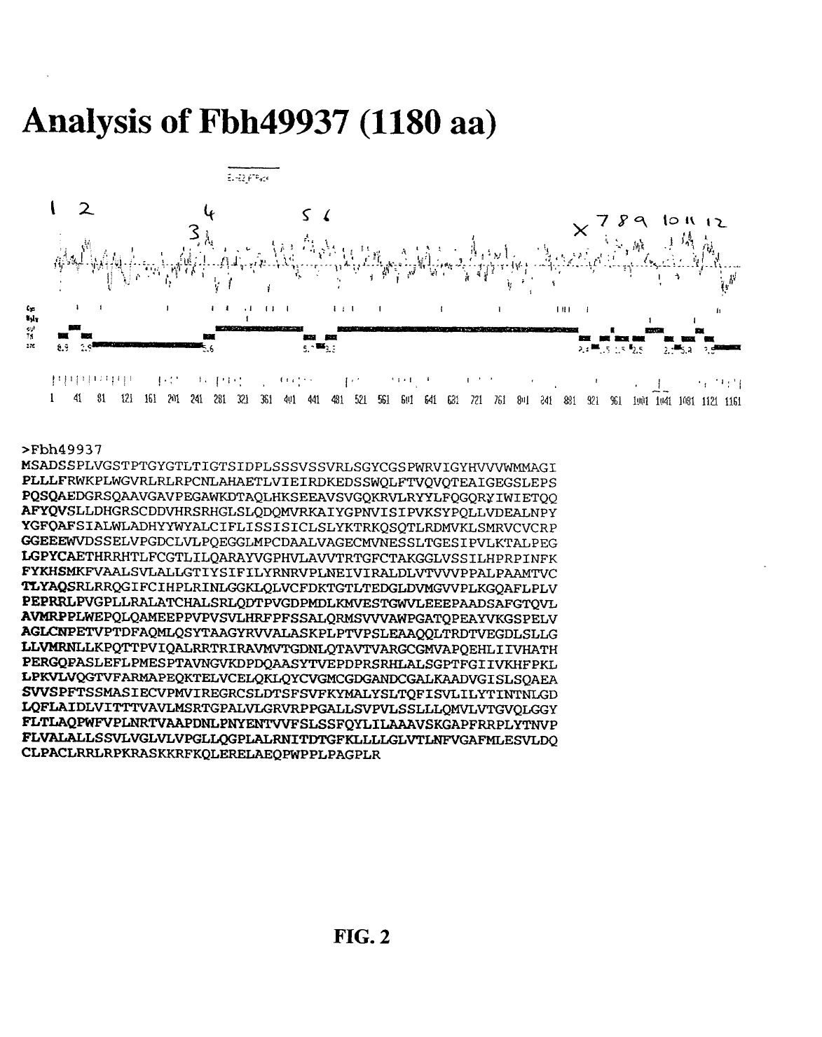

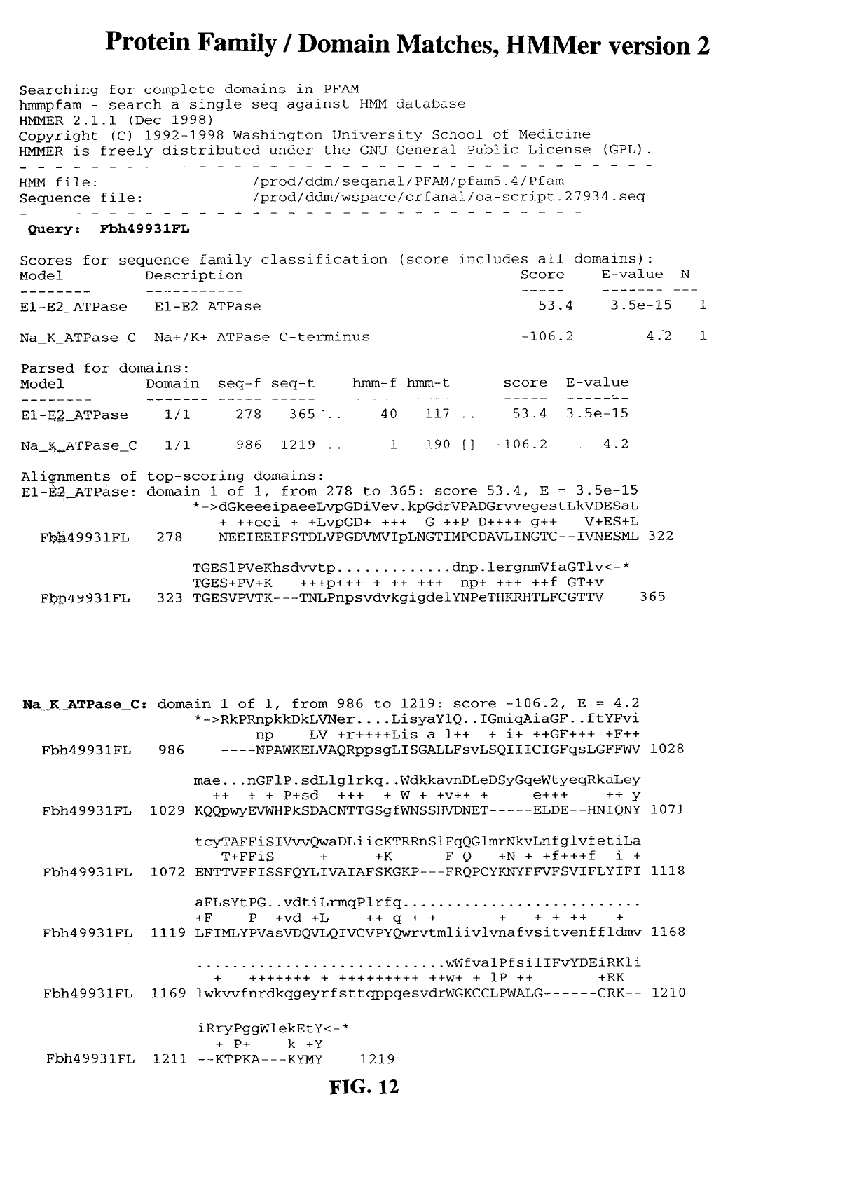

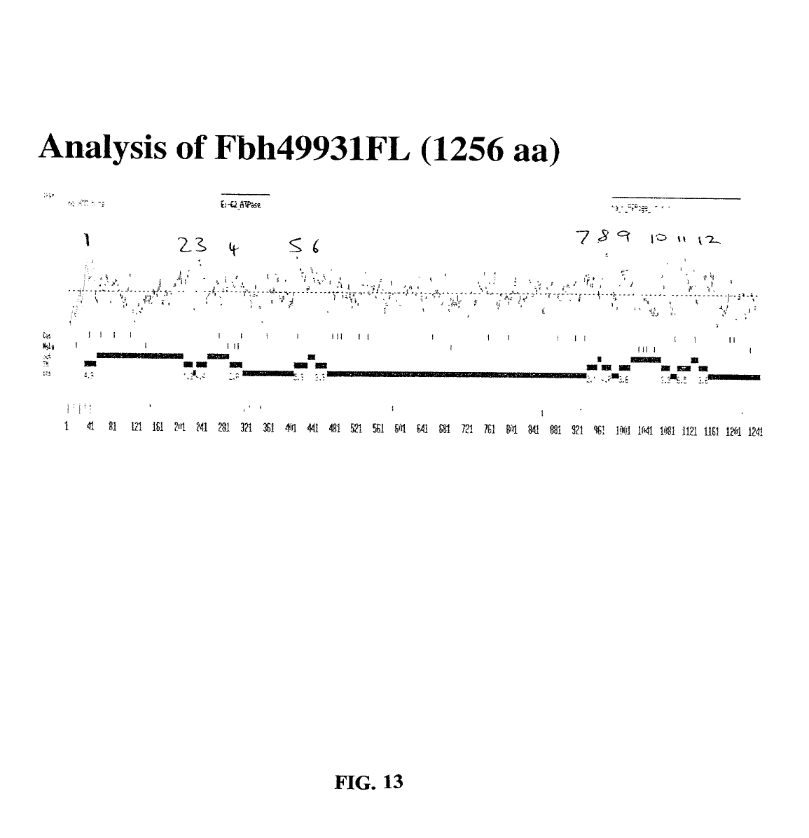

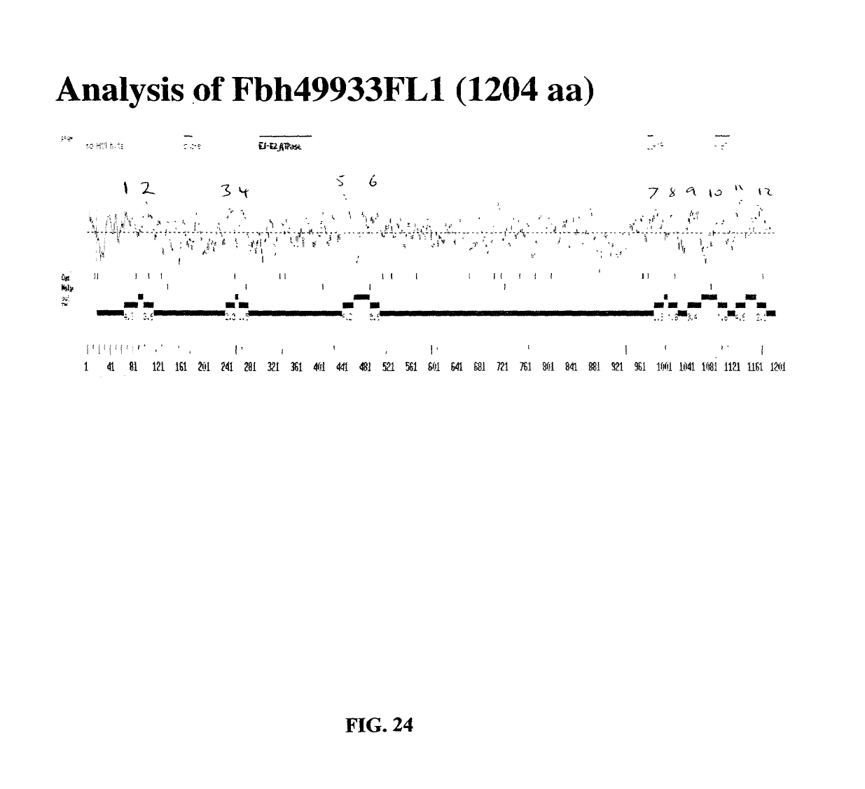

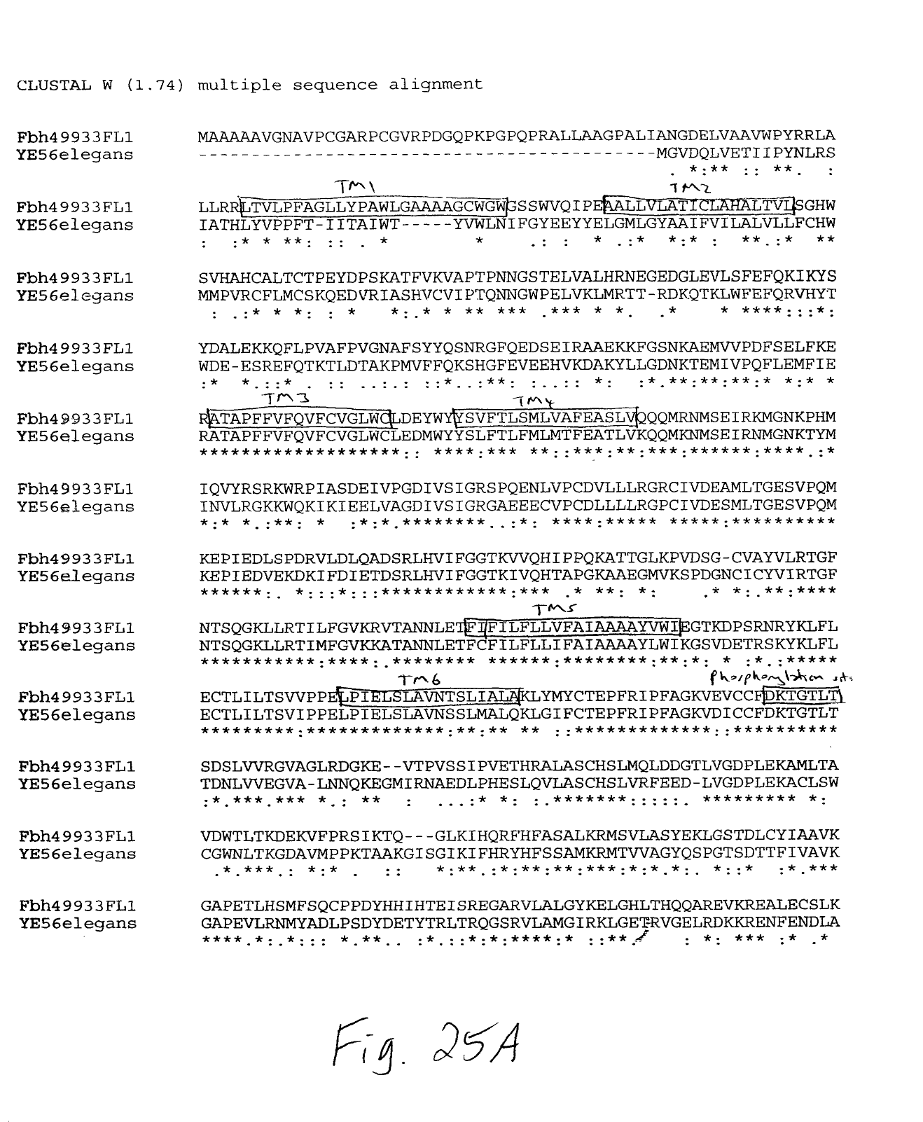

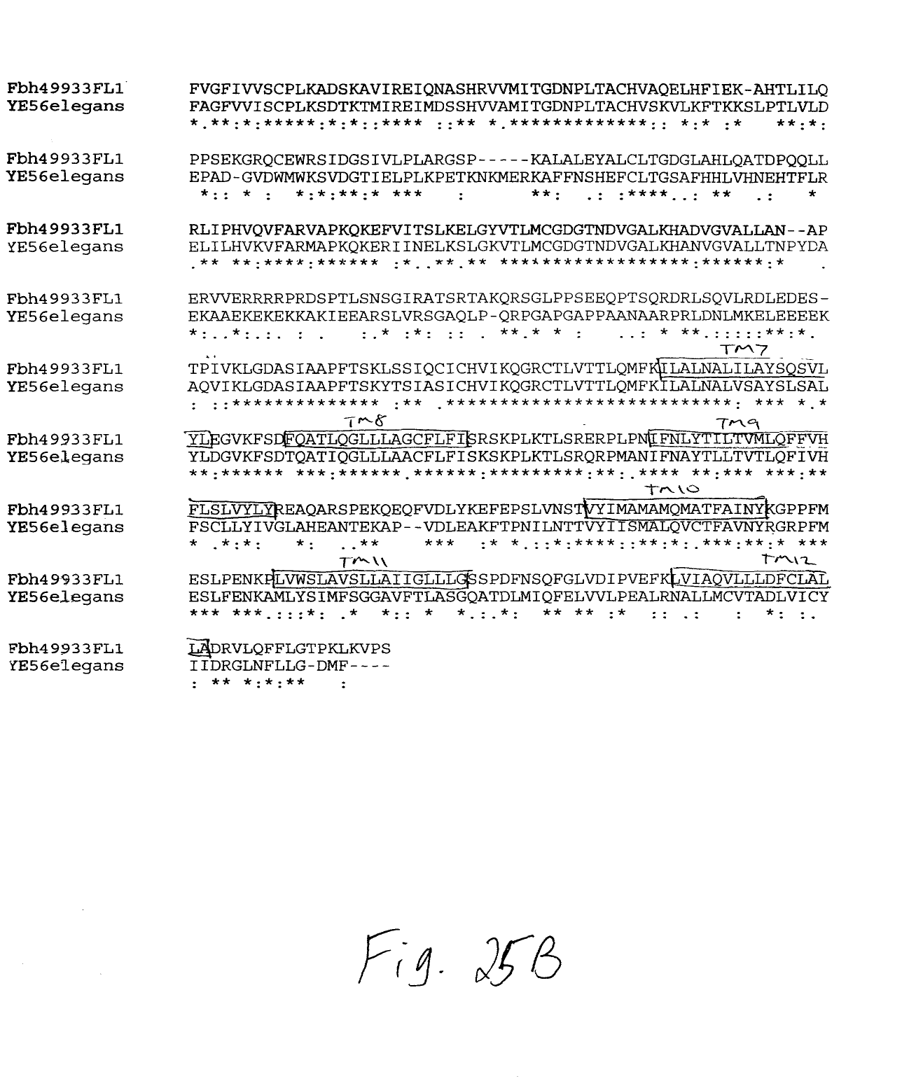

For example, the family of HEAT proteins of the present invention comprises at least one “transmembrane domain,” preferably at least 2, 3, or 4 transmembrane domains, more preferably 5, 6, 7, 8, or 9 transmembrane domains, even more preferably 10 or 11 transmembrane domains, and most preferably, 12 transmembrane domains. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta, W. N. et al. (1996) Annu. Rev. Neurosci. 19:235-263, the contents of which are incorporated herein by reference. Amino acid residues 8-25, 47-65, 231-253, 256-276, 428-448, 464-484, 936-954, 963-987, 994-1015, 1049-1065, 1079-1102, and 1118-1134 of the human HEAT-1 protein (SEQ ID NO:2) are predicted to comprise transmembrane domains (see FIG. 2). Amino acid residues 29-50, 211-227, 234-253, 294-317, 410-434, 449-469, 941-960, 968-985, 1000-1020, 1076-1092, 1105-1129, 1144-1160 of the human HEAT-2 protein (SEQ ID NO:6) are predicted to comprise transmembrane domains (see FIGS. 12 and 13). Amino acid residues 65-89, 99-116, 242-258, 265-281, 445-464, 493-509, 990-1007, 1015-1031, 1049-1073, 1103-1119, 1134-1151, 1171-1187 of the human HEAT-3 protein (SEQ ID NO:9) are also predicted to comprise transmembrane domains (see FIGS. 24 and 25).

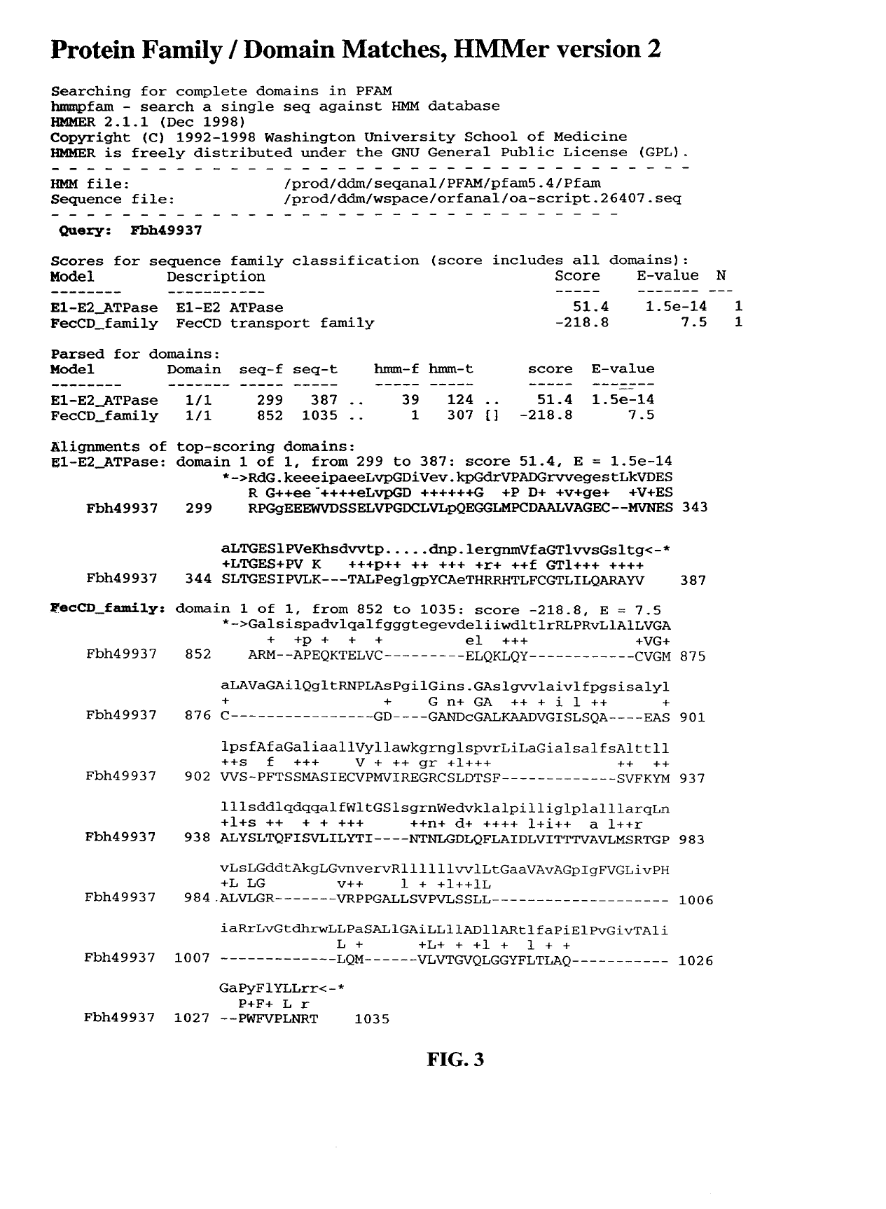

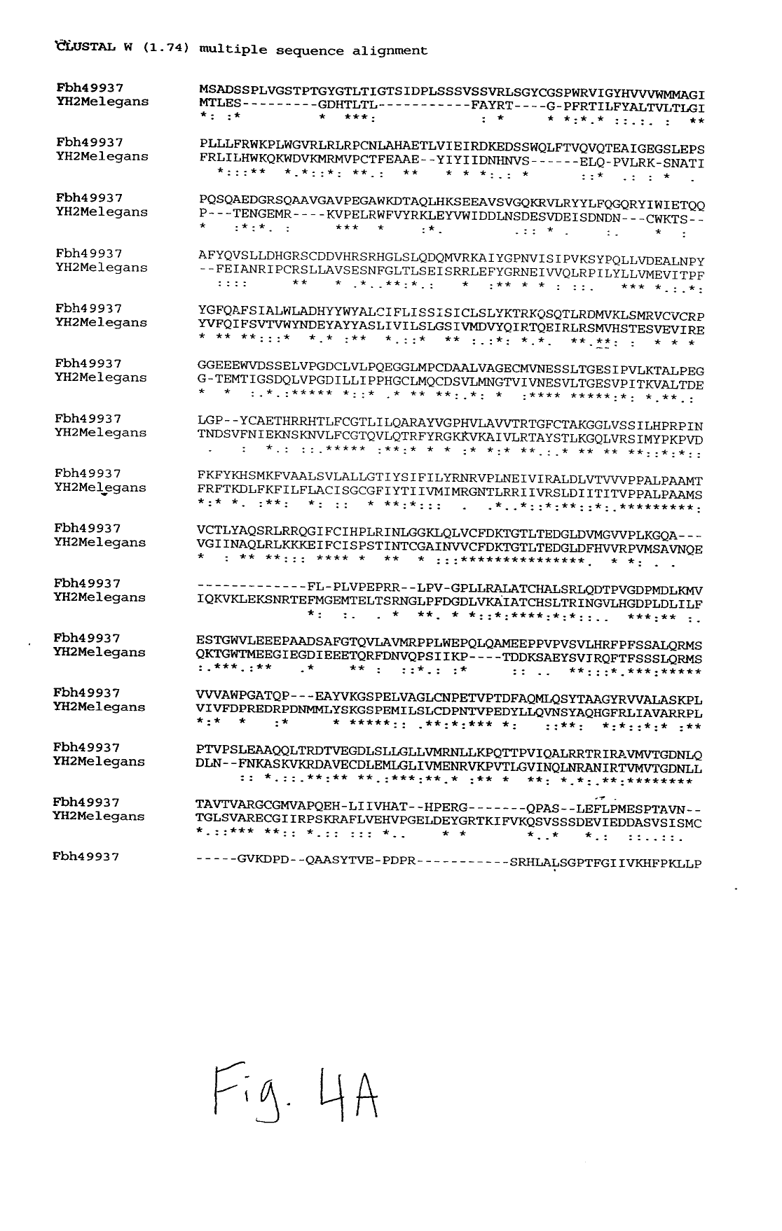

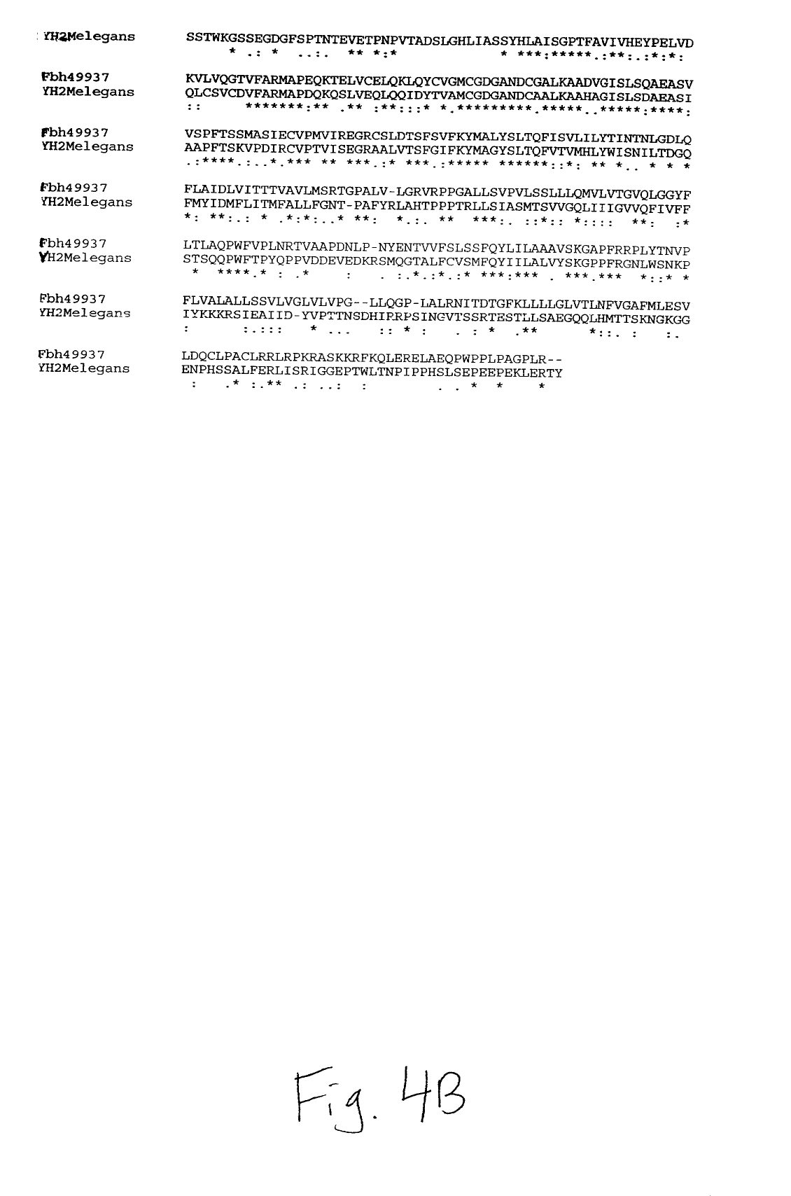

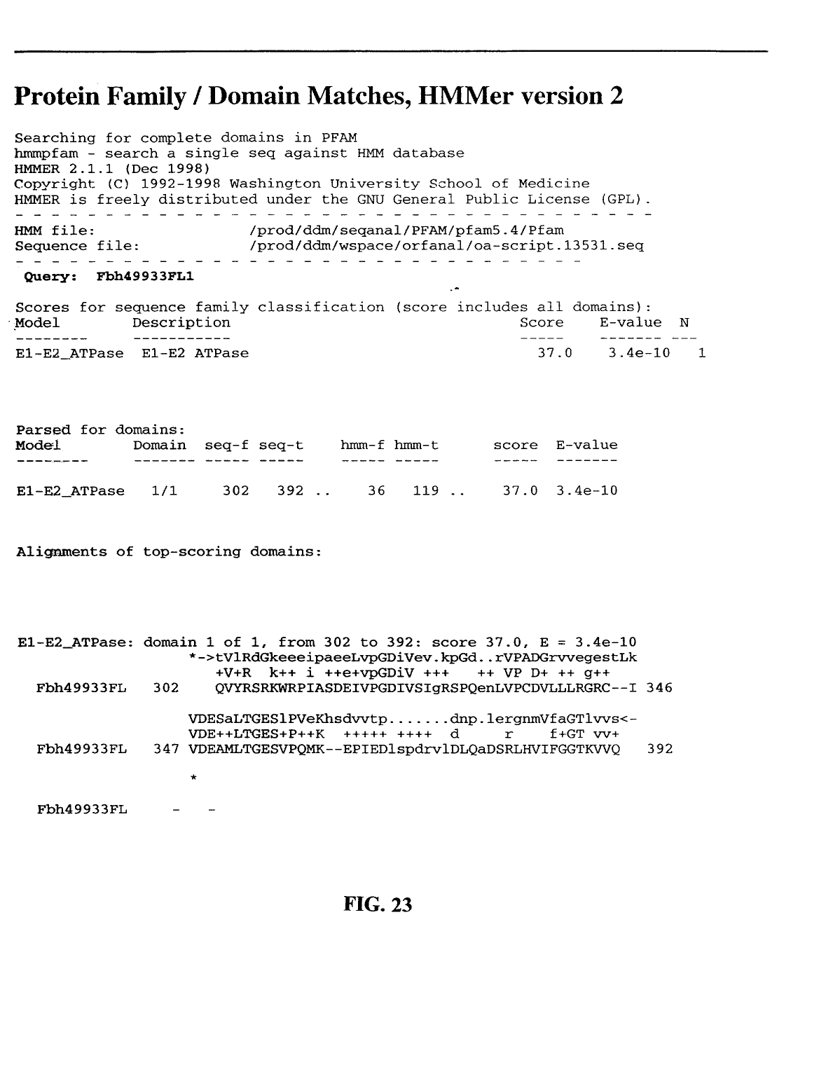

In another embodiment, members of the HEAT family of proteins include at least one “E1-E2 ATPase domain” in the protein or corresponding nucleic acid molecule. As used herein, the term “E1-E2 ATPase” domain includes a protein domain having at least about 70-110 amino acid residues and a bit score of at least 30 when compared against an E1-E2 ATPase Hidden Markov Model (HMM), e.g., PFAM Accession Number PF00122. Preferably, an E1-E2 ATPase domain includes a protein having an amino acid sequence of about 80-100, or more preferably about 87, 89, or 90 amino acid residues, and a bit score of at least 35, 40, 50, or more preferably, 37.0, 51.4, or 53.4. To identify the presence of an E1-E2 ATPase domain in a HEAT protein, and make the determination that a protein of interest has a particular profile, the amino acid sequence of the protein is searched against a database of known protein motifs and/or domains (e.g., the HMM database). The E1-E2 ATPase domain (HMM) has been assigned the PFAM Accession number PF00122 (see the PFAM website, available online through Washington University in Saint Louis). A search was performed against the HMM database resulting in the identification of an E1-E2 ATPase domain in the amino acid sequence of human HEAT-1 at about residues 299-387 of SEQ ID NO:2. The results of the search are set forth in FIG. 3. A search was also performed against the HMM database resulting in the identification of an E1-E2 ATPase domain in the amino acid sequence of human HEAT-2 at about residues 278-365 of SEQ ID NO:6. The results of the search are set forth in FIG. 12. A search was further performed against the HMM database resulting in the identification of an E1-E2 ATPase domain in the amino acid sequence of human HEAT-3 at about residues 302-392 of SEQ ID NO:9. The results of the search are set forth in FIG. 23.

A description of the Pfam database can be found in Sonhammer et al. (1997) Proteins 28:405-420, and a detailed description of HMMs can be found, for example, in Gribskov et al. (1990) Methods Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference.

Preferably an E1-E2 ATPase domain is at least about 70-110 amino acid residues and has an “E1-E2 ATPase activity”, for example, the ability to interact with a HEAT substrate or target molecule (e.g., ATP or a cation such as Ca²⁺); to transport a HEAT substrate or target molecule (e.g., a cation such as Ca²⁺) from one side of a biological membrane to the other; to adopt an E1 conformation or an E2 conformation; to convert a HEAT substrate or target molecule to a product (e.g., to hydrolyze ATP); to interact with a second non-HEAT protein; to modulate intra- or inter-cellular signaling and/or gene transcription (e.g., either directly or indirectly); to modulate vascular smooth muscle tone; to modulate cellular growth and/or proliferation; and/or to modulate angiogenesis. Accordingly, identifying the presence of an “E1-E2 ATPase domain” can include isolating a fragment of a HEAT molecule (e.g., a HEAT polypeptide) and assaying for the ability of the fragment to exhibit one of the aforementioned E1-E2 ATPase domain activities.

In another embodiment, a HEAT molecule of the present invention may also be identified based on its ability to adopt an E1 conformation or an E2 conformation. As used herein, an “E1 conformation” of a HEAT protein includes a 3-dimensional conformation of a HEAT protein which does not exhibit HEAT activity (e.g., the ability to transport Ca²⁺), as defined herein. An E1 conformation of a HEAT protein usually occurs when the HEAT protein is unphosphorylated. As used herein, an “E2 conformation” of a HEAT protein includes a 3-dimensional conformation of a HEAT protein which exhibits HEAT activity (e.g., the ability to transport c Ca²⁺), as defined herein. An E2 conformation of a HEAT protein usually occurs when the HEAT protein is phosphorylated.

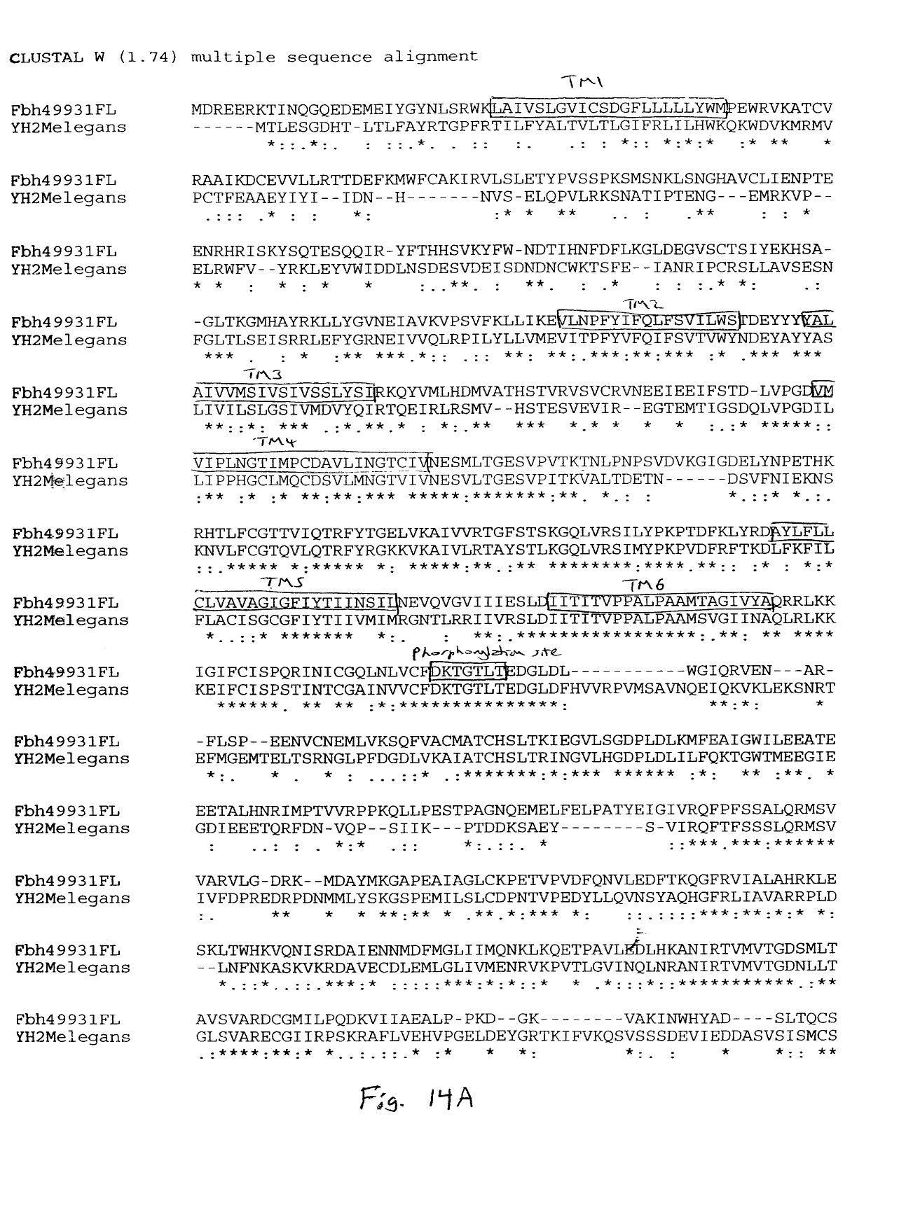

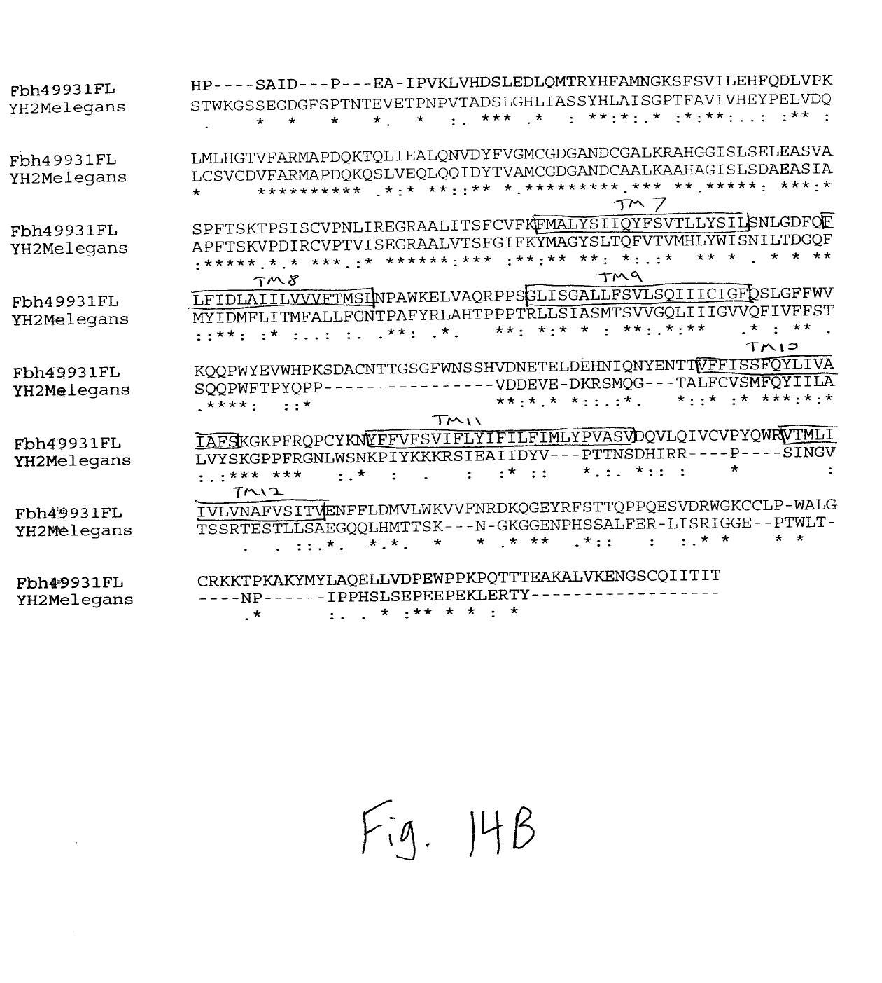

In another embodiment, a HEAT protein of the present invention is identified based on the presence of an “E1-E2 ATPases phosphorylation site” in the protein or corresponding nucleic acid molecule. An E1-E2 ATPases phosphorylation site functions in accepting a phosphate moiety and has the following consensus sequence: D-K-T-G-T-[LIVM]-[TI] (SEQ ID NO:11), wherein D is phosphorylated. The use of amino acids in brackets indicates that the amino acid at the indicated position may be any one of the amino acids within the brackets, e.g., [TI] indicates any of one of either T (threonine) or I (isoleucine). The E1-E2 ATPases phosphorylation site has been assigned ProSite Accession Number PS00154. To identify the presence of an E1-E2 ATPases phosphorylation site in a HEAT protein, and to make the determination that a protein of interest has a particular profile, the amino acid sequence of the protein may be searched against a database of known protein domains (e.g., the ProSite database) using the default parameters (available online through the Swiss Institute for Bioinformatics). A search was performed against the ProSite database resulting in the identification of an E1-E2 ATPases phosphorylation site in the amino acid sequence of human HEAT-1 (SEQ ID NO:2) at about residues 513-519. A similar search resulted in the identification of an E1-E2 phosphorylation site in the amino acid sequence of human HEAT-2 (SEQ ID NO:6) at about residues 498-504 (see FIGS. 14A-14B) and in the amino acid sequence of human HEAT-3 (SEQ ID NO:9) at about residues 533-539 (see FIGS. 25A-25B).

Preferably an E1-E2 ATPases phosphorylation site has a “phosphorylation site activity,” for example, the ability to be phosphorylated; to be dephosphorylated; to regulate the E1-E2 conformational change cf the HEAT protein in which it is contained; to regulate transport of Ca²⁺ across a biological membrane by the HEAT protein in which it is contained; and/or to regulate the activity (as defined herein) of the HEAT protein in which it is contained. Accordingly, identifying the presence of an “E1-E2 ATPases phosphorylation site” can include isolating a fragment of a HEAT molecule (e.g., a HEAT polypeptide) and assaying for the ability of the fragment to exhibit one of the aforementioned phosphorylation site activities.

The family of HEAT proteins of the present invention also comprises at least one “large extramembrane domain” in the protein or corresponding nucleic acid molecule. As used herein, a “large extramembrane domain” includes a domain having greater than 20 amino acid residues that is found between transmembrane domains, preferably on the cytoplasmic side of the plasma membrane, and does not span or traverse the plasma membrane. A large extramembrane domain preferably includes at least one, two, three, four or more motifs or consensus sequences characteristic of P-type or E1-E2 ATPases, i. e., includes one, two, three, four, or more “P-type ATPase consensus sequences or motifs”. As used herein, the phrase “P-type ATPase consensus sequences or motifs” includes any consensus sequence or motif known in the art to be characteristic of P-type ATPases, including, but not limited to, the P-type ATPase sequence 1 motif (as defined herein), the P-type ATPase sequence 2 motif (as defined herein), the P-type ATPase sequence 3 motif (as defined herein), and the E1-E2 ATPases phosphorylation site (as defined herein).

In one embodiment, the family of HEAT proteins of the present invention comprises at least one “N-terminal” large extramembrane domain in the protein or corresponding nucleic acid molecule. As used herein, an “N-terminal” large extramembrane domain is found in the N-terminal ⅓^{rd} of the protein, preferably between the fourth and fifth transmembrane domains of a HEAT protein, and includes about 50-270, 50-250, 60-230, 70-210, 80-190, 90-170, or preferably, 92, 151, or 163 amino acid residues. In a preferred embodiment, an N-terminal large extramembrane domain includes at least one P-type ATPase sequence 1 motif (as described herein). An N-terminal large extramembrane domain was identified in the amino acid sequence of human HEAT-1 at about residues 277-427 of SEQ ID NO:2. An N-terminal large extramembrane domain was also identified in the amino acid sequence of human HEAT-2 at about residues 318-409 of SEQ ID NO:6 and in the amino acid sequence of human HEAT-3 at about residues 282-444 of SEQ ID NO:9.

The family of HEAT proteins of the present invention also comprises at least one “C-terminal” large extramembrane domain in the protein or corresponding nucleic acid molecule. As used herein, a “C-terminal” large extramembrane domain is found in the C-terminal ⅔^{rds} of the protein, preferably between the sixth and seventh transmembrane domains of a HEAT protein and includes about 340-590, 360-570, 380-550, 400-530, 420-510, 440-490, or preferably, 451, 471, or 480 amino acid residues. In a preferred embodiment, a C-terminal large extramembrane domain includes at least one or more of the following motifs: a P-type ATPase sequence 2 motif (as described herein), a P-type ATPase sequence 3 motif (as defined herein), and/or an E1-E2 ATPases phosphorylation site (as defined herein). A C-terminal large extramembrane domain was identified in the amino acid sequence of human HEAT-1 at about residues 485-935 of SEQ ID NO:2, in the amino acid sequence of human HEAT-2 at about residues 470-940 of SEQ ID NO:6, and in the amino acid sequence of human HEAT-3 at about residues 510-989 of SEQ ID NO:9.

In another embodiment, a HEAT protein of the present invention includes at least one “P-type ATPase sequence 1 motif” in the protein or corresponding nucleic acid molecule. As used herein, a “P-type ATPase sequence 1 motif” is a conserved sequence motif diagnostic for P-type ATPases (Tang, X. et al. (1996) Science 272:1495-1497; Fagan, M. J. and Saier, M. H. (1994) J. Mol. Evol. 38:57). A P-type ATPase sequence 1 motif is involved in the coupling of ATP hydrolysis with transport (e.g., transport of Ca²⁺). The consensus sequence for a P-type ATPase sequence 1 motif is [DNS]-[QENR]-[SA]-[LIVSAN]-[LIV]-[TSN]-G-E-[SN] (SEQ ID NO:13). The use of amino acids in brackets indicates that the amino acid at the indicated position may be any one of the amino acids within the brackets, e.g., [SA] indicates any of one of either S (serine) or A (alanine). In a preferred embodiment, a P-type ATPase sequence 1 motif is contained within an N-terminal large extramembrane domain. In another preferred embodiment, a P-type ATPase sequence 1 motif in the HEAT proteins of the present invention has at least 1, 2, 3, 4, 5, 6, 7, 8 or more amino acid resides which match the consensus sequence for a P-type ATPase sequence 1 motif. A P-type ATPase sequence 1 motif was identified in the amino acid sequence of human HEAT-1 at about residues 341-349 of SEQ ID NO:2, in the amino acid sequence of human HEAT-2 at about residues 318-326 of SEQ ID NO:6, and in the amino acid sequence of human HEAT-3 at about residues 348-356 of SEQ ID NO:9.

In another embodiment, a HEAT protein of the present invention includes at least one “P-type ATPase sequence 2 motif” in the protein or corresponding nucleic acid molecule. As used herein, a “P-type ATPase sequence 2 motif” is a conserved sequence motif diagnostic for P-type ATPases (Tang, X. et al. (1996) Science 272:1495-1497; Fagan, M. J. and Saier, M. H. (1994) J. Mol. Evol. 38:57). Preferably, a P-type ATPase sequence 2 motif overlaps with and/or includes an E1-E2 ATPases phosphorylation site (as defined herein). The consensus sequence for a P-type ATPase sequence 2 motif is [LIV]-[CAML]-[STFL]-D-K-T-G-T-[LI]-T (SEQ ID NO:14). The use of amino acids in brackets indicates that the amino acid at the indicated position may be any one of the amino acids within the brackets, e.g., [LI] indicates any of one of either L (leucine) or I (isoleucine). In a preferred embodiment, a P-type ATPase sequence 2 motif is contained within a C-terminal large extramembrane domain. In another preferred embodiment, a P-type ATPase sequence 2 motif in the HEAT proteins of the present invention has at least 1, 2, 3, 4, 5, 6, 7, 8, 9 or more amino acid resides which match the consensus sequence for a P-type ATPase sequence 2 motif. A P-type ATPase sequence 2 motif was identified in the amino acid sequence of human HEAT-1 at about residues 510-519 of SEQ ID NO:2, in the amino acid sequence of human HEAT-2 at about residues 495-504 of SEQ ID NO:6, and in the amino acid sequence of human HEAT-3 at about residues 530-539 of SEQ ID NO:9.

In yet another embodiment, a HEAT protein of the present invention includes at least one “P-type ATPase sequence 3 motif” in the protein or corresponding nucleic acid molecule. As used herein, a “P-type ATPase sequence 3 motif” is a conserved sequence motif diagnostic for P-type ATPases (Tang, X. et al. (1996) Science 272:1495-1497; Fagan, M. J. and Saier, M. H. (1994) J. Mol. Evol. 38:57). A P-type ATPase sequence 3 motif is involved in ATP binding. The consensus sequence for a P-type ATPase sequence 3 motif is [TIV]-G-D-G-X-N-D-[ASG]-P-[ASV]-L (SEQ ID NO:15). X indicates that the amino acid at the indicated position may be any amino acid (i.e., is not conserved). The use of amino acids in brackets indicates that the amino acid at the indicated position may be any one of the amino acids within the brackets, e.g., [TIV] indicates any of one of either T (threonine), I (isoleucine), or V (valine). In a preferred embodiment, a P-type ATPase sequence 3 motif is contained within a C-terminal large extramembrane domain. In another preferred embodiment, a P-type ATPase sequence 3 motif in the HEAT proteins of the present invention has at least 1, 2, 3, 4, 5, 6, 7, 8 or more amino acid resides (including the amino acid at the position indicated by “X”) which match the consensus sequence for a P-type ATPase sequence 3 motif. A P-type ATPase sequence 3 motif was identified in the amino acid sequence of human HEAT-1 at about residues 876-886 of SEQ ID NO:2, in the amino acid sequence of human HEAT-2 at about residues 881-891 of SEQ ID NO:6, and in the amino acid sequence of human HEAT-3 at about residues 862-872 of SEQ ID NO:9.

Isolated HEAT proteins of the present invention have an amino acid sequence sufficiently homologous to the amino acid sequence of SEQ ID NO:2, 6, or 9, or are encoded by a nucleotide sequence sufficiently homologous to SEQ ID NO:1, 3, 5, 7, 8, or 10. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains having at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more homology or identity across the amino acid sequences of the domains and contain at least one and preferably two structural domains or motifs, are defined herein as sufficiently homologous. Furthermore, amino acid or nucleotide sequences which share at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more homology or identity and share a common functional activity are defined herein as sufficiently homologous.

In a preferred embodiment, a HEAT protein includes at least one or more of the following domains or motifs: a transmembrane domain, an E1-E2 ATPase domain, an E1-E2 ATPases phosphorylation site, an N-terminal large extramembrane domain, a C-terminal large extramembrane domain, a P-type ATPase sequence 1 motif, a P-type ATPase sequence 2 motif, and/or a P-type ATPase sequence 3 motif, and has an amino acid sequence at least about 50%, 55%, 60%, 65%, 70%, 75%, 80%, 85%, 90%, 91%, 92%, 93%, 94%, 95%, 96%, 97%, 98%, 99%, 99.1% 99.2%, 99.3%, 99.4%, 99.5%, 99.6%, 99.7%, 99.8%, 99.9% or more homologous or identical to the amino acid sequence of SEQ ID NO:2, 6, or 9, or the amino acid sequence encoded by the DNA insert of the plasmid deposited with ATCC as Accession Number ______, ______, or ______. In yet another preferred embodiment, a HEAT protein includes at least one or more of the following domains or motifs: a transmembrane domain, an E1-E2 ATPase domain, an E1-E2 ATPases phosphorylation site, an N-terminal large extramembrane domain, a C-terminal large extramembrane domain, a P-type ATPase sequence 1 motif, a P-type ATPase sequence 2 motif, and/or a P-type ATPase sequence 3 motif, and is encoded by a nucleic acid molecule having a nucleotide sequence which hybridizes under stringent hybridization conditions to a complement of a nucleic acid molecule comprising the nucleotide sequence of SEQ ID NO:1, 3, 5, 7, 8, or 10. In another preferred embodiment, a HEAT protein includes at least one or more of the following domains or motifs: a transmembrane domain, an E1-E2 ATPase domain, an E1-E2 ATPases phosphorylation site, an N-terminal large extramembrane domain, a C-terminal large extramembrane domain, a P-type ATPase sequence 1 motif, a P-type ATPase sequence 2 motif, and/or a P-type ATPase sequence 3 motif, and has a HEAT activity.

As used interchangeably herein, a “HEAT activity”, “biological activity of HEAT” or “functional activity of HEAT”, includes an activity exerted or mediated by a HEAT protein, polypeptide or nucleic acid molecule on a HEAT responsive cell or on a HEAT substrate, as determined in vivo or in vitro, according to standard techniques. In one embodiment, a HEAT activity is a direct activity, such as an association with a HEAT target molecule. As used herein, a “target molecule” or “binding partner” is a molecule with which a HEAT protein binds or interacts in nature, such that HEAT-mediated function is achieved. A HEAT target molecule can be a non-HEAT molecule or a HEAT protein or polypeptide of the present invention. In an exemplary embodiment, a HEAT target molecule is a HEAT substrate (e.g., a Ca²⁺ ion; ATP; or a non-HEAT protein). A HEAT activity can also be an indirect activity, such as a cellular signaling activity mediated by interaction of the HEAT protein with a HEAT substrate (e.g., regulation of vascular smooth muscle tone, cellular growth and/or proliferation, and/or angiogenesis).

In a preferred embodiment, a HEAT activity is at least one of the following activities: (i) interaction with a HEAT substrate or target molecule (e.g., a Ca²⁺ ion; ATP; or a non-HEAT protein); (ii) transport of a HEAT substrate or target molecule (e.g., a Ca²⁺ ion) from one side of a biological membrane to the other; (iii) the ability to be phosphorylated or dephosphorylated; (iv) adoption of an E1 conformation or an E2 conformation; (v) conversion of a HEAT substrate or target molecule to a product (e.g., hydrolysis of ATP to ADP and free phosphate); (vi) interaction with a second non-HEAT protein; (vii) modulation of intra- or inter-cellular signaling and/or gene transcription (e.g., either directly or indirectly); (viii) modulation of vascular smooth muscle tone; (ix) modulation of cellular growth and/or proliferation; and/or (x) modulation of angiogenesis.

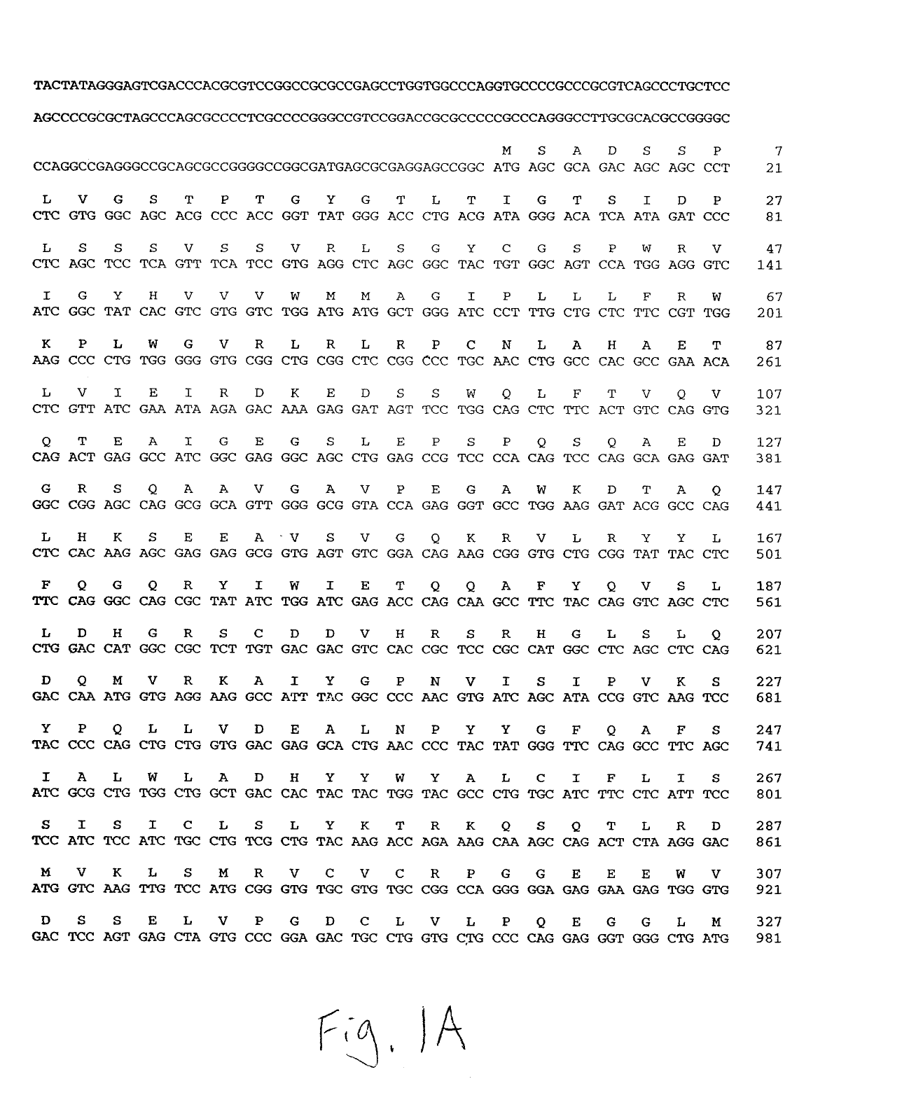

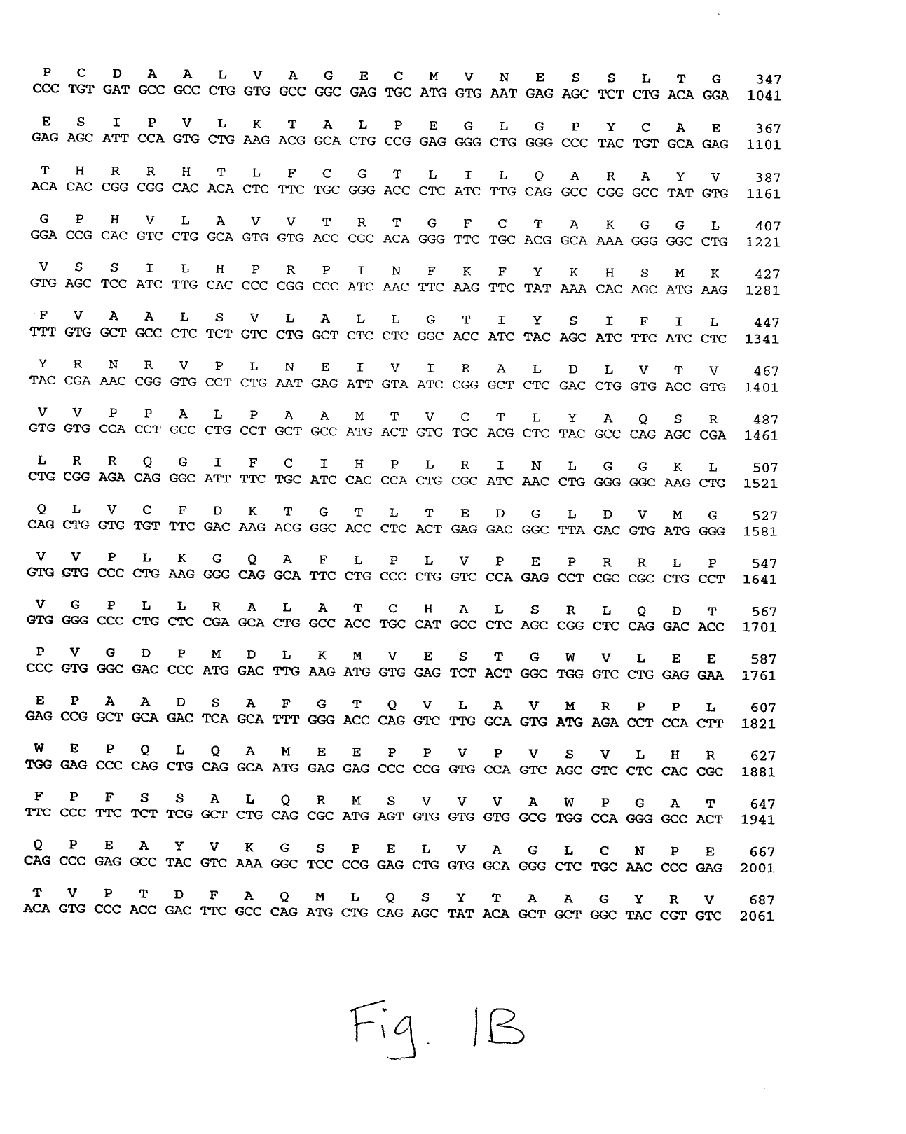

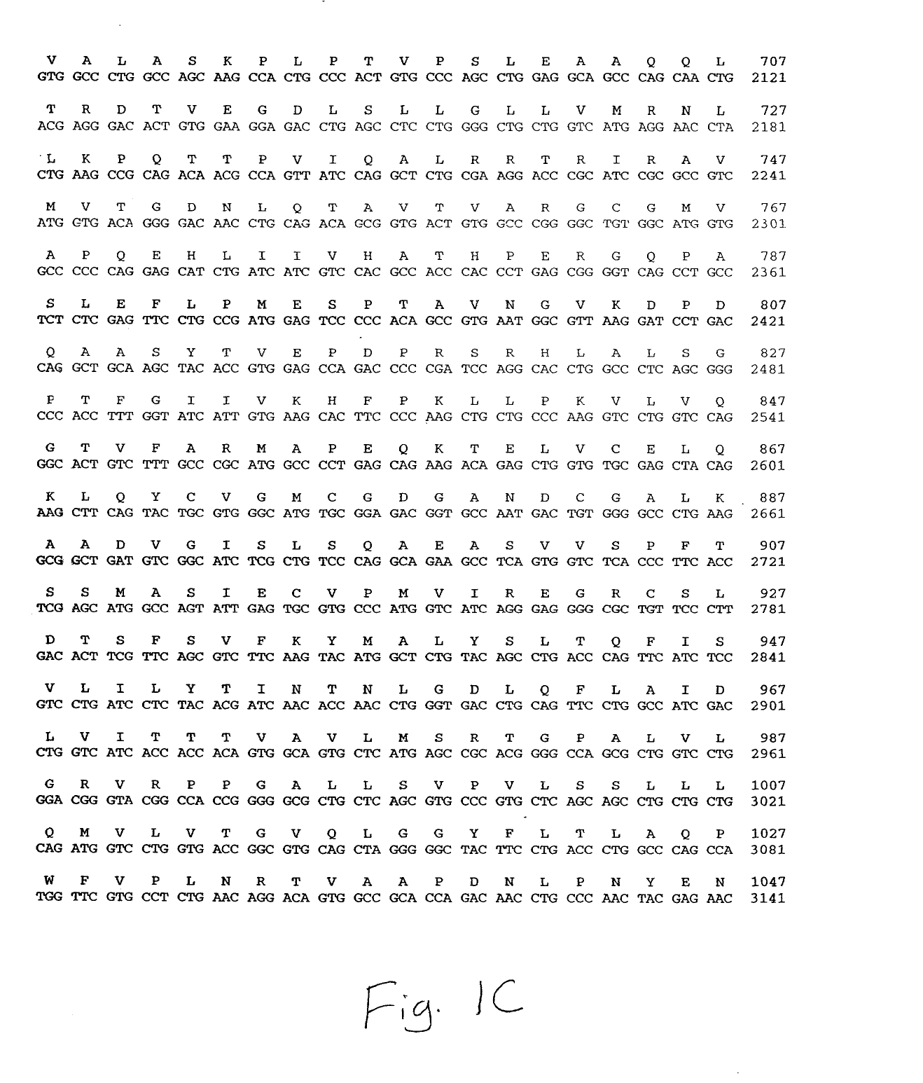

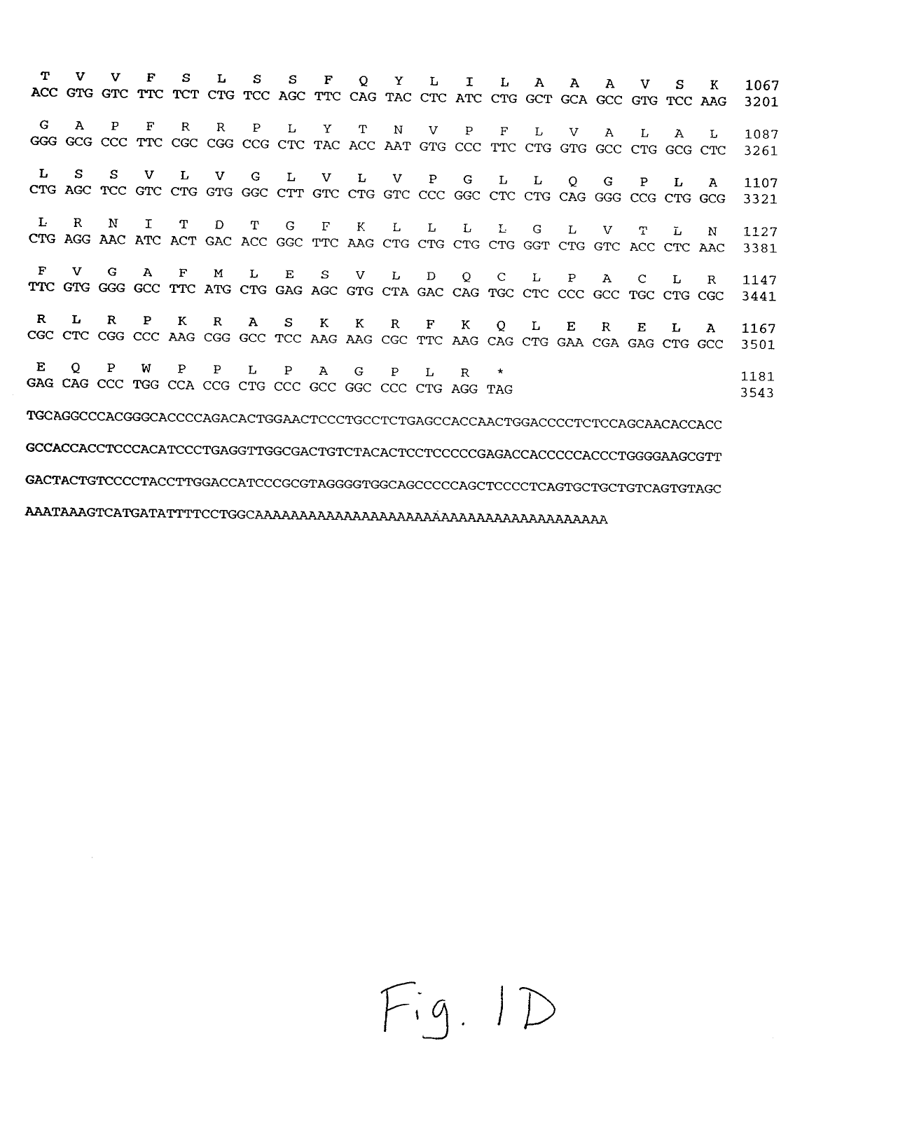

The nucleotide sequence of the isolated human HEAT-1 cDNA and the predicted amino acid sequence encoded by the HEAT-1 cDNA are shown in FIGS. 1A-1D and in SEQ ID NOs:1 and 2, respectively. A plasmid containing the human HEAT-1 cDNA was deposited with the American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit were made merely as a convenience for those of skill in the art and is not an admission that a deposit is required under 35 U.S.C. §112.

The human HEAT-1 gene, which is approximately 4055 nucleotides in length, encodes a protein having a molecular weight of approximately 129.8 kD and which is approximately 1180 amino acid residues in length.

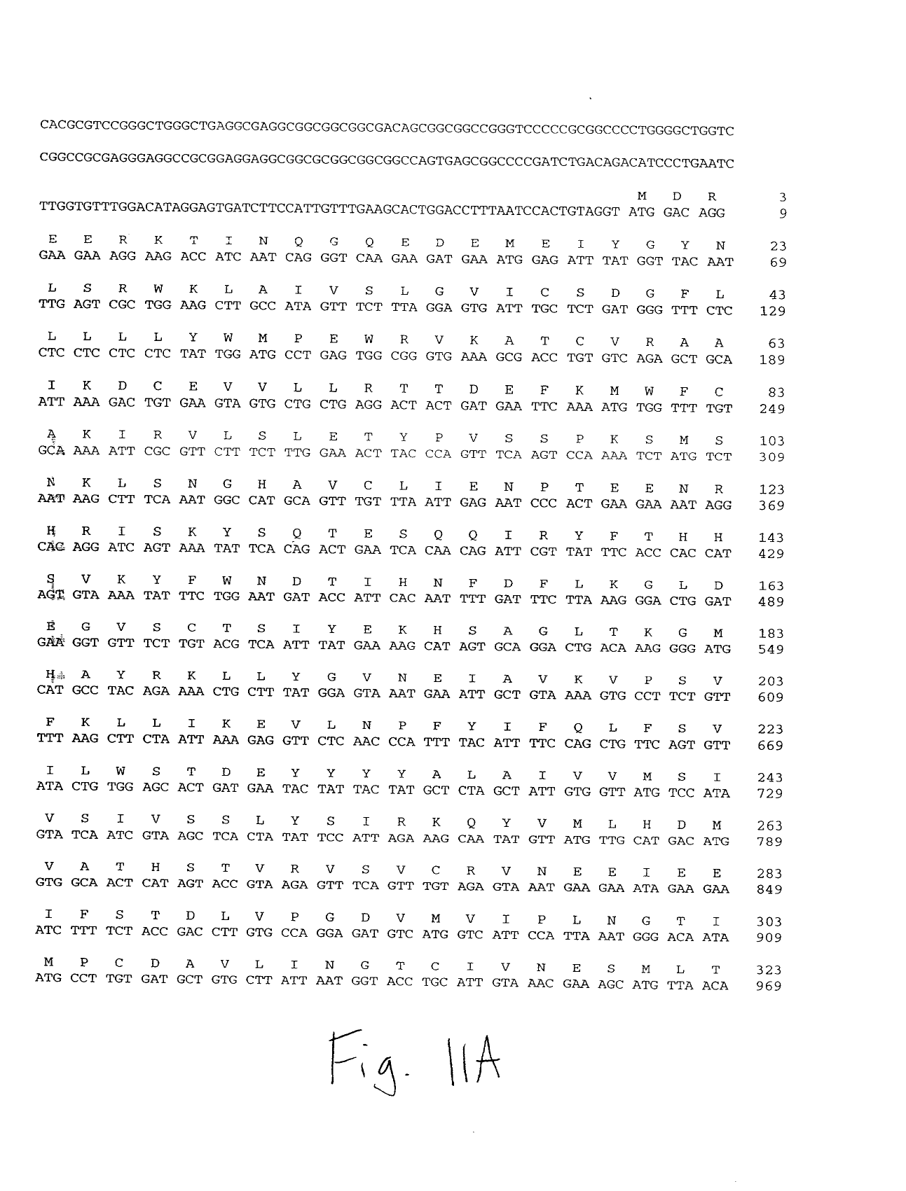

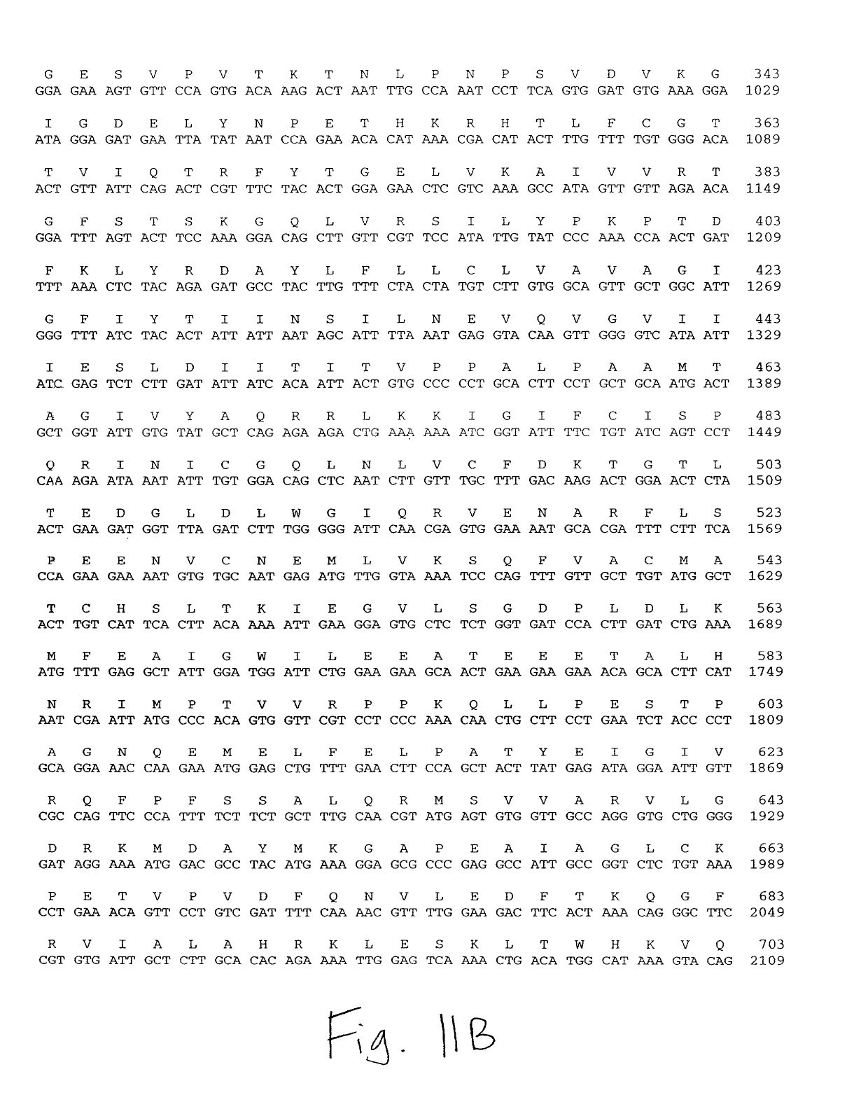

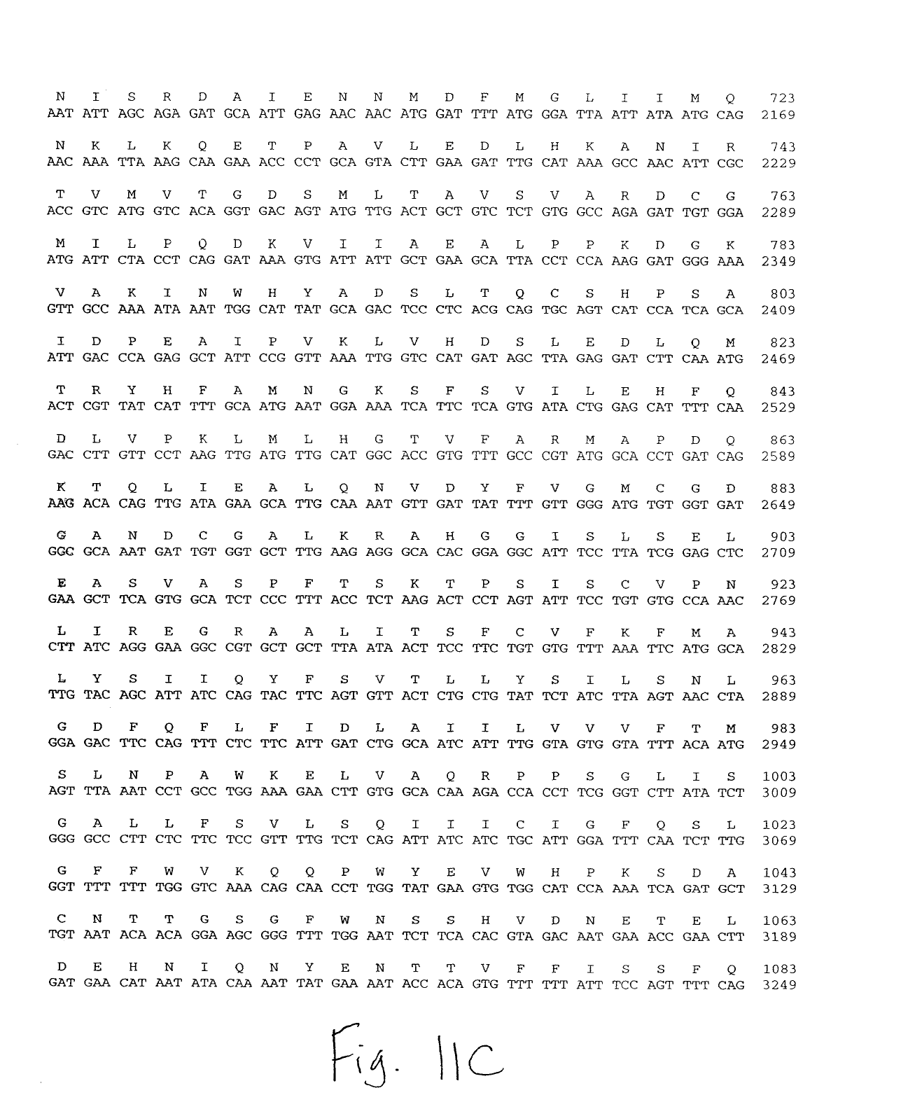

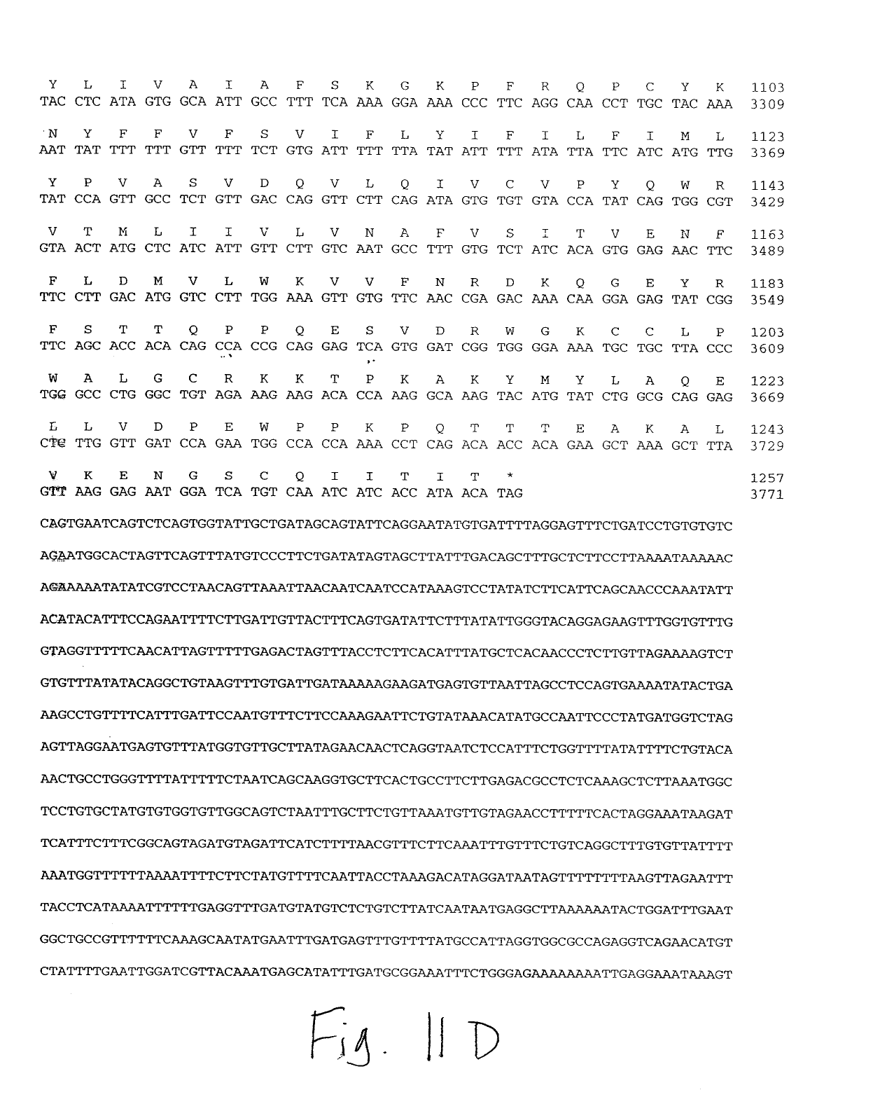

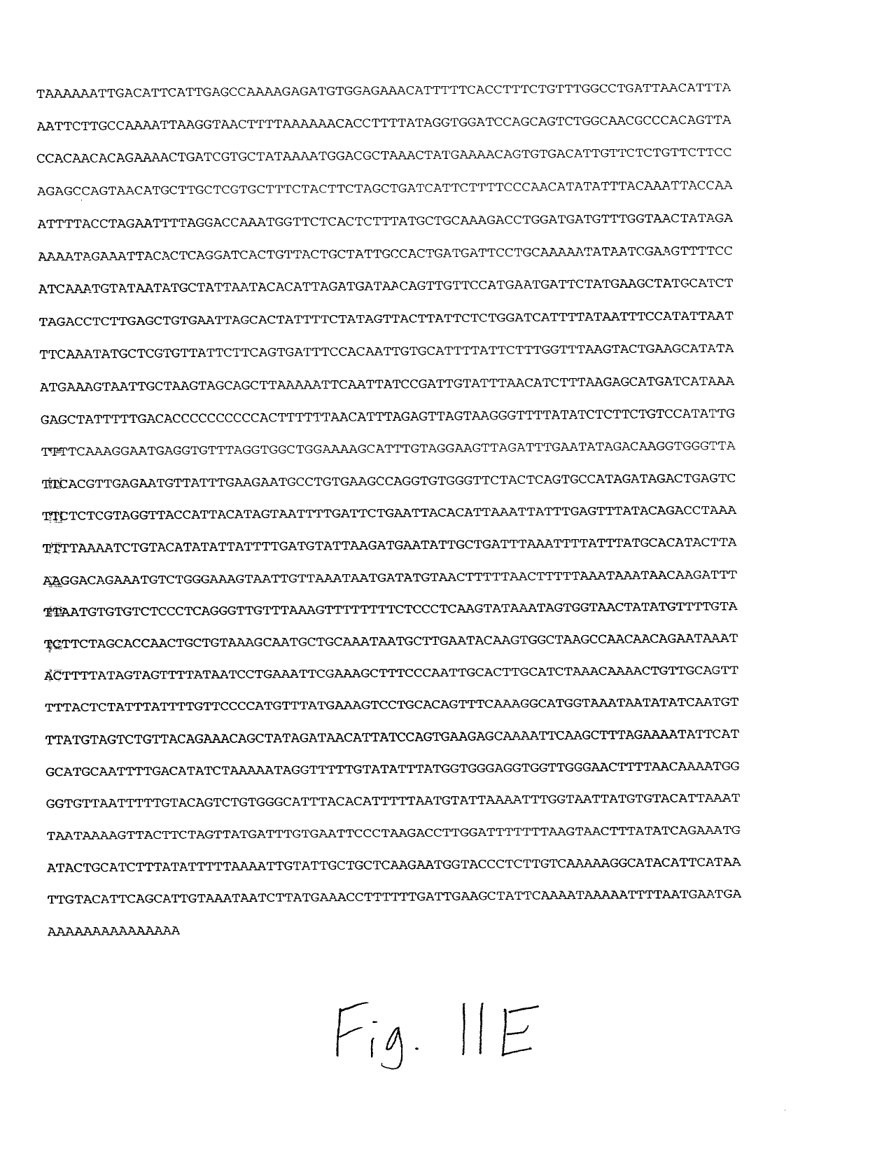

The nucleotide sequence of the isolated human HEAT-2 cDNA and the predicted amino acid sequence encoded by the HEAT-2 cDNA are shown in FIGS. 11A-11E and in SEQ ID NOs:5 and 6, respectively. A plasmid containing the human HEAT-2 cDNA was deposited with the American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit were made merely as a convenience for those of skill in the art and is not an admission that a deposit is required under 35 U.S.C. §112.

The human HEAT-2 gene, which is approximately 7249 nucleotides in length, encodes a protein having a molecular weight of approximately 138.2 kD and which is approximately 1256 amino acid residues in length.

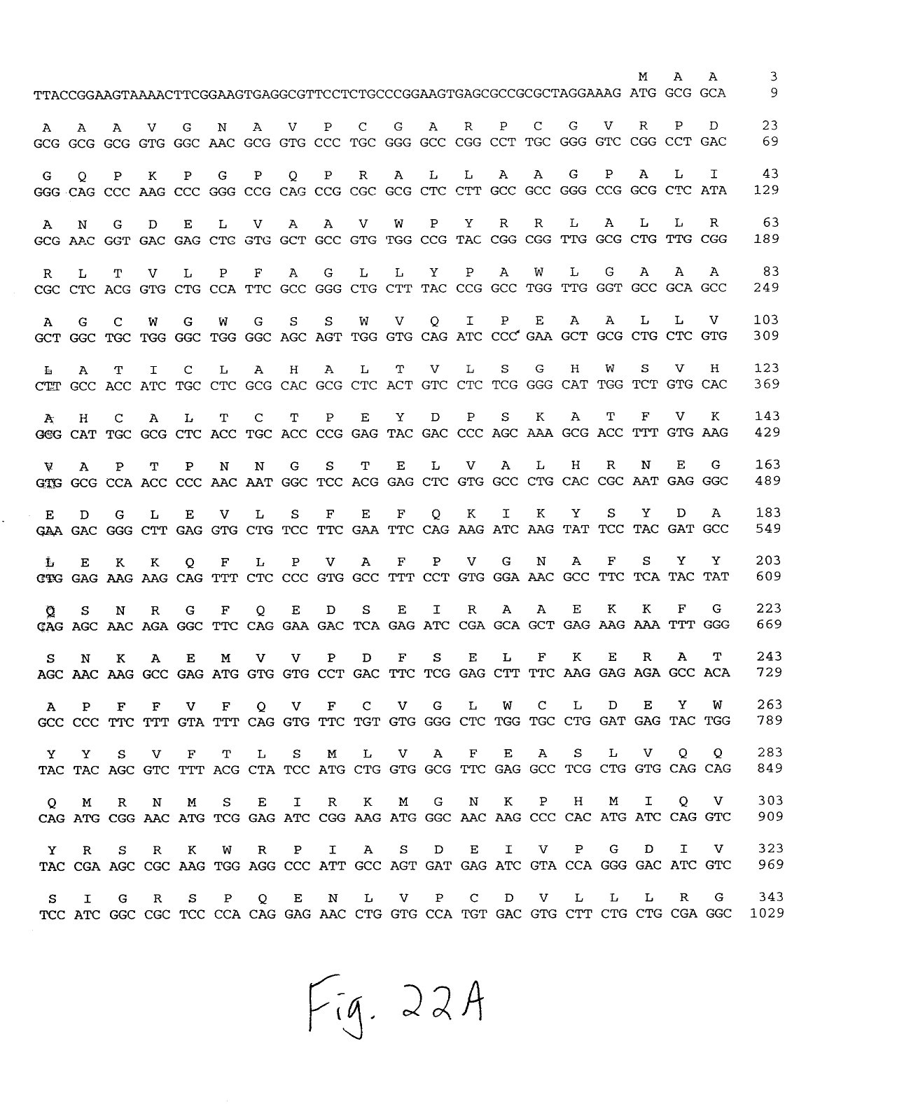

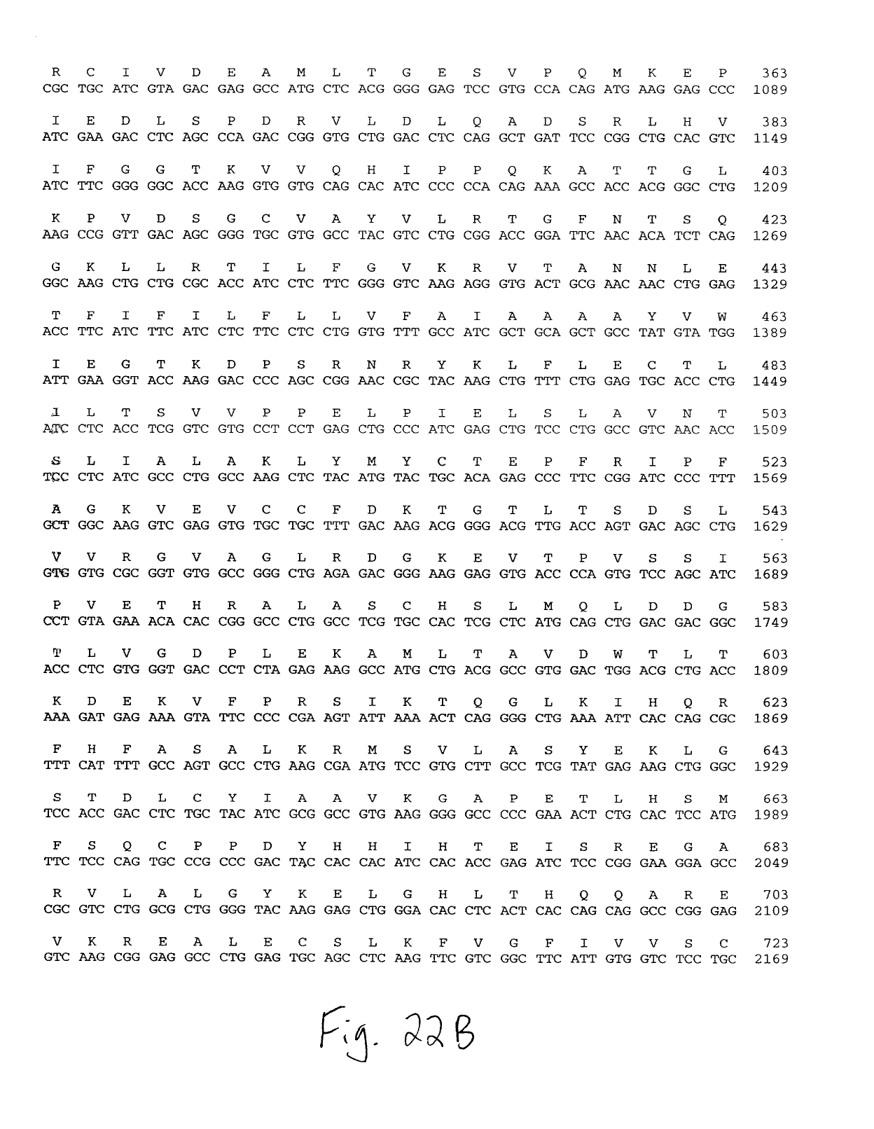

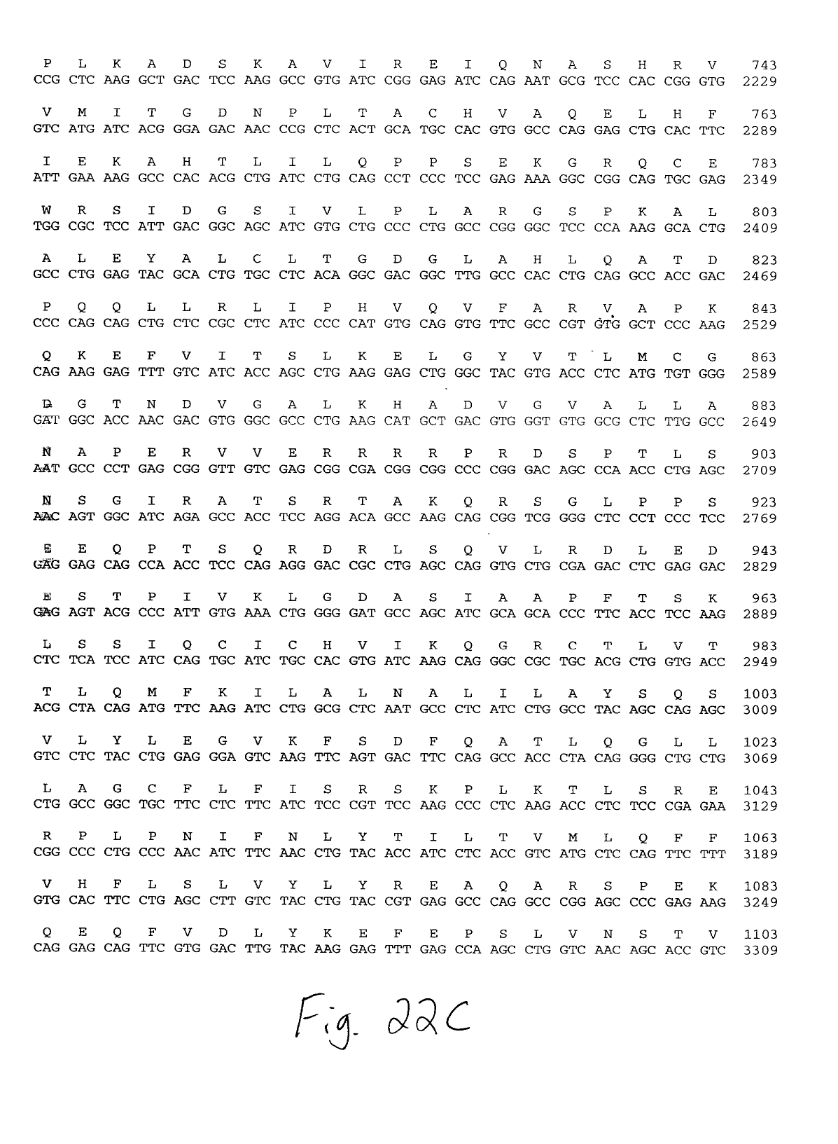

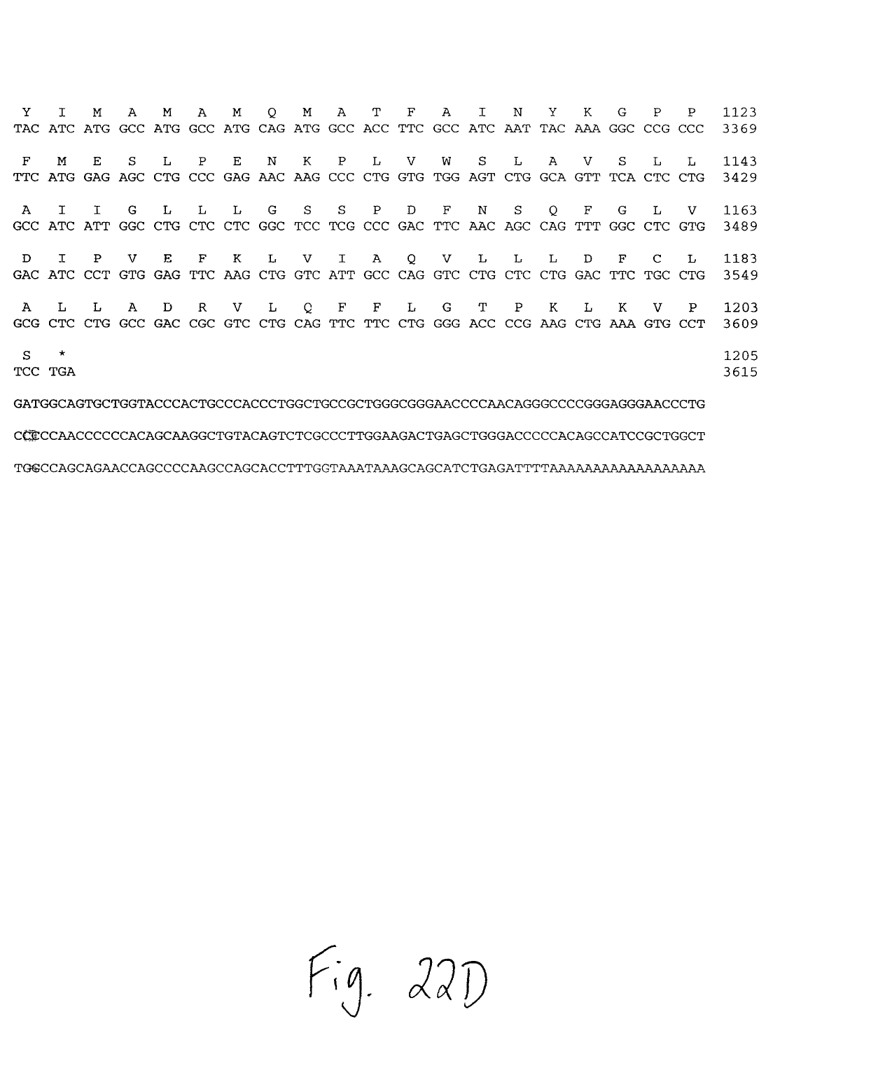

The nucleotide sequence of the isolated human HEAT-3 cDNA and the predicted amino acid sequence encoded by the HEAT-3 cDNA are shown in FIGS. 22A-22D and in SEQ ID NOs:8 and 9, respectively. A plasmid containing the human HEAT-3 cDNA was deposited with the American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit were made merely as a convenience for those of skill in the art and is not an admission that a deposit is required under 35 U.S.C. §112.

The human HEAT-3 gene, which is approximately 3919 nucleotides in length, encodes a protein having a molecular weight of approximately 132.5 kD and which is approximately 1204 amino acid residues in length.

Various aspects of the invention are described in further detail in the following subsections:

I. Isolated Nucleic Acid Molecules

One aspect of the invention pertains to isolated nucleic acid molecules that encode HEAT proteins or biologically active portions thereof, as well as nucleic acid fragments sufficient for use as hybridization probes to identify HEAT-encoding nucleic acid molecules (e.g., HEAT mRNA) and fragments for use as PCR primers for the amplification or mutation of HEAT nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated HEAT nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, 3, 5, 7, 8, or 10, or the nucleotide sequence of the DNA insert of the plasmid deposited with ATCC as Accession Number ______, ______, or ______, or a portion thereof, can be isolated using standard molecular biology techniques and the sequence information provided herein. Using all or a portion of the nucleic acid sequence of SEQ ID NO:1, 3, 5, 7, 8, or 10, or the nucleotide sequence of the DNA insert of the plasmid deposited with ATCC as Accession Number ______, ______, or ______, as hybridization probes, HEAT nucleic acid molecules can be isolated using standard hybridization and cloning techniques (e.g., as described in Sambrook, J. et al., Molecular Cloning: A Laboratory Manual. 2^{nd} ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3, 5, 7, 8, or 10, or the nucleotide sequence of the DNA insert of the plasmid deposited with ATCC as Accession Number ______, ______, or ______ can be isolated by the polymerase chain reaction (PCR) using synthetic oligonucleotide primers designed based upon the sequence of SEQ ID NO:1, 3, 5, 7, 8, or 10, or the nucleotide sequence of the DNA insert of the plasmid deposited with ATCC as Accession Number ______, ______, or ______.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to HEAT nucleotide sequences can be prepared by standard synthetic techniques, e.g., using an automated DNA synthesizer.

In one embodiment, an isolated nucleic acid molecule of the invention comprises the nucleotide sequence shown in SEQ ID NO:1, 3, 5, 7, 8, or 10. This cDNA may comprise sequences encoding the human HEAT-1 protein (e.g., the “coding region”, from nucleotides 210-3749), as well as 5′ untranslated sequences (nucleotides 1-209) and 3′ untranslated sequences (nucleotides 3750-4055) of SEQ ID NO:1. The cDNA may also comprise sequences encoding human the HEAT-2 protein (e.g., the “coding region”, from nucleotides 225-3992), as well as 5′ untranslated sequences (nucleotides 1-224) and 3′ untranslated sequences (nucleotides 3993-7249) of SEQ ID NO:5. The cDNA may also comprise sequences encoding the human HEAT-3 protein (e.g., the “coding region”, from nucleotides 68-3679), as well as 5′ untranslated sequences (nucleotides 1-67) and 3′ untranslated sequences (nucleotides 3680-3919) of SEQ ID NO:8. Alternatively, the nucleic acid molecule can comprise only the coding region of SEQ ID NO:1 (e.g., nucleotides 210-3749, corresponding to SEQ ID NO:3), SEQ ID NO:5 (e.g., nucleotides 225-3992, corresponding to SEQ ID NO:7), or SEQ ID NO:8 (e.g., nucleotides 68-3679, corresponding to SEQ ID NO:10). Accordingly, in another embodiment, an isolated nucleic acid molecule of the invention comprises SEQ ID NO:3 and nucleotides 1-209 of SEQ ID NO:1. In another embodiment, the isolated nucleic acid molecule comprises SEQ ID NO:3 and nucleotides 3750-4055 of SEQ ID NO:1. In yet another embodiment, an isolated nucleic acid molecule of the invention comprises SEQ ID NO:7 and nucleotides 1-224 of SEQ ID NO:5. In another embodiment, the isolated nucleic acid molecule comprises SEQ ID NO:7 and nucleotides 3993-7249 of SEQ ID NO:5. In still another embodiment, the isolated nucleic acid molecule comprises SEQ ID NO:10 and nucleotides 1-67 of SEQ ID NO:8. In another embodiment, the isolated nucleic acid molecule comprises SEQ ID NO:10 and nucleotides 3680-3919 of SEQ ID NO:8. In yet another embodiment, the nucleic acid molecule consists of the nucleotide sequence set forth as SEQ ID NO:1, SEQ ID NO:3, SEQ ID NO:5, SEQ ID NO:7, SEQ ID NO:8, OR SEQ ID NO:10. In still another embodiment, the nucleic acid molecule can comprise the coding region of SEQ ID NO:1 (e.g., nucleotides 210-3749, corresponding to SEQ ID NO:3), as well as a stop codon (e.g., nucleotides 3750-3752 of SEQ ID NO:1). In another embodiment, the nucleic acid molecule can comprise the coding region of SEQ ID NO:5 (e.g., nucleotides 225-3992, corresponding to SEQ ID NO:7), as well as a stop codon (e.g., nucleotides 3993-3995 of SEQ ID NO:5). In another embodiment, the nucleic acid molecule can comprise the coding region of SEQ ID NO:8 (e.g., nucleotides 68-3679, corresponding to SEQ ID NO:10), as well as a stop codon (e.g. nucleotides 3680-3682 of SEQID NO:5). In yet other embodiments, the nucleic acid molecule can comprise nucleotides 1-28 of SEQ ID NO:1, nucleotides 4016-4055 of SEQ ID NO:1, nucleotides 1-175 of SEQ ID NO:5, nucleotides 410-1225 of SEQ ID NO:5, or nucleotides 224-462 of SEQ ID NO:8.

In still another embodiment, an isolated nucleic acid molecule of the invention comprises a nucleic acid molecule which is a complement of the nucleotide sequence shown in SEQ ID NO:1, 3, 5, 7, 8, or 10, or the nucleotide sequence of the DNA insert of the plasmid deposited with ATCC as Accession Number ______, ______, or ______, or a portion of any of these nucleotide sequences. A nucleic acid molecule which is complementary to the nucleotide sequence shown in SEQ ID NO:1, 3, 5, 7, 8, or 10, or the nucleotide sequence of the DNA insert of the plasmid deposited with ATCC as Accession Number ______, ______, or ______, is one which is sufficiently complementary to the nucleotide sequence shown in SEQ ID NO:1, 3, 5, 7, 8, or 10, or the nucleotide sequence of the DNA insert of the plasmid deposited with ATCC as Accession Number ______, ______, or ______, such that it can hybridize to the nucleotide sequence shown in SEQ ID NO:1, 3, 5, 7, 8, or 10, or the nucleotide sequence of the DNA insert of the plasmid deposited with ATCC as Accession Number ______, ______, or ______, thereby forming a stable duplex.

In still another embodiment, an isolated nucleic acid molecule of the present invention comprises a nucleotide sequence which is at least about 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to the nucleotide sequence shown in SEQ ID NO:1, 3, 5, 7, 8, or 10 (e.g., to the entire length of the nucleotide sequence), or to the nucleotide sequence (e.g., the entire length of the nucleotide sequence) of the DNA insert of the plasmid deposited with ATCC as Accession Number ______, ______, or ______, or a portion or complement of any of these nucleotide sequences. In one embodiment, a nucleic acid molecule of the present invention comprises a nucleotide sequence which is at least (or no greater than) 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1250, 1300, 1350, 1400, 1450, 1500, 1550, 1600, 1650, 1700, 1750, 1800, 1850, 1900, 1950, 1994, 2000, 2050, 2073, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3441, 3450, 3500, 3550, 3600, 3650, 3700, 3750, 3800, 3841, 3850, 3900, 3950; 4000, 4050, 4100, 4150, 4200, 4250, 4300, 4350, 4400, 4450, 4500, 4550, 4600, 4650, 4700, 4750, 4800, 4850, 4900, 4950, 5000, 5050, 5100, 5150, 5200, 5250, 5300, 5350, 5400, 5450, 5500, 5550, 5600, 5650, 5700, 5750, 5800, 5850, 5900, 5950, 6000, 6050, 6100, 6150, 6200, 6250, 6300, 6350, 6400, 6450, 6500, 6550, 6600, 6650, 6700, 6750, 6800, 6850, 6900, 6950, 7000, 7050, 7100, 7150, 7200 or more nucleotides in length and hybridizes under stringent hybridization conditions to a complement of a nucleic acid molecule of SEQ ID NO:1, 3, 5, 7, 8, or 10, or the nucleotide sequence of the DNA insert of the plasmid deposited with ATCC as Accession Number _____, ______, or ______.

Moreover, the nucleic acid molecule of the invention can comprise only a portion of the nucleic acid sequence of SEQ ID NO:1, 3, 5, 7, 8, or 10, or the nucleotide sequence of the DNA insert of the plasmid deposited with ATCC as Accession Number ______, ______, or ______, for example, a fragment which can be used as a probe or primer or a fragment encoding a portion of a HEAT protein, e.g., a biologically active portion of a HEAT protein. The nucleotide sequence determined from the cloning of the HEAT gene allows for the generation of probes and primers designed for use in identifying and/or cloning other HEAT family members, as well as HEAT homologues from other species. The probe/primer (e.g., oligonucleotide) typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, 3, 5, 7, 8, or 10, or the nucleotide sequence of the DNA insert of the plasmid deposited with ATCC as Accession Number ______, ______, or ______, of an anti-sense sequence of SEQ ID NO:1, 3, 5, 7, 8, or 10, or the nucleotide sequence of the DNA insert of the plasmid deposited with ATCC as Accession Number ______, ______, or ______, or of a naturally occurring allelic variant or mutant of SEQ ID NO:1, 3, 5, 7, 8, or 10, or the nucleotide sequence of the DNA insert of the plasmid deposited with ATCC as Accession Number ______, ______, or ______.

Exemplary probes or primers are at least (or no greater than) 12 or 15, 20 or 25, 30, 35, 40, 45, 50, 55, 60, 65, 70, 75 or more nucleotides in length and/or comprise consecutive nucleotides of an isolated nucleic acid molecule described herein. Also included within the scope of the present invention are probes or primers comprising contiguous or consecutive nucleotides of an isolated nucleic acid molecule described herein, but for the difference of 1, 2, 3, 4, 5, 6, 7, 8, 9 or 10 bases within the probe or primer sequence. Probes based on the HEAT nucleotide sequences can be used to detect (e.g., specifically detect) transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. In another embodiment a set of primers is provided, e.g., primers suitable for use in a PCR, which can be used to amplify a selected region of a HEAT sequence, e.g., a domain, region, site or other sequence described herein. The primers should be at least 5, 10, or 50 base pairs in length and less than 100, or less than 200, base pairs in length. The primers should be identical, or differ by no greater than 1, 2, 3, 4, 5, 6, 7, 8, 9 or 10 bases when compared to a sequence disclosed herein or to the sequence of a naturally occurring variant. Such probes can be used as a part of a diagnostic test kit for identifying cells or tissue which misexpress a HEAT protein, such as by measuring a level of a HEAT-encoding nucleic acid in a sample of cells from a subject, e.g., detecting HEAT mRNA levels or determining whether a genomic HEAT gene has been mutated or deleted.

A nucleic acid fragment encoding a “biologically active portion of a HEAT protein” can be prepared by isolating a portion of the nucleotide sequence of SEQ ID NO:1, 3, 5, 7, 8, or 10, or the nucleotide sequence of the DNA insert of the plasmid deposited with ATCC as Accession Number ______, ______, or ______, which encodes a polypeptide having a HEAT biological activity (the biological activities of the HEAT proteins are described herein), expressing the encoded portion of the HEAT protein (e.g., by recombinant expression in vitro) and assessing the activity of the encoded portion of the HEAT protein. In an exemplary embodiment, the nucleic acid molecule is at least 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1250, 1300, 1350, 1400, 1450, 1500, 1550, 1600, 1650, 1700, 1750, 1800, 1850, 1900, 1950, 1994, 2000, 2050, 2073, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3441, 3450, 3500, 3550, 3600, 3650, 3700, 3750, 3800, 3841, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 5000, 5050, 5100, 5150, 5200, 5250, 5300, 5350, 5400, 5450, 5500, 5550, 5600, 5650, 5700, 5750, 5800, 5850, 5900, 5950, 6000, 6050, 6100, 6150, 6200, 6250, 6300, 6350, 6400, 6450, 6500, 6550, 6600, 6650, 6700, 6750, 6800, 6850, 6900, 6950, 7000, 7050, 7100, 7150, 7200 or more nucleotides in length and encodes a protein having a HEAT activity (as described herein).

The invention further encompasses nucleic acid molecules that differ from the nucleotide sequence shown in SEQ ID NO:1, 3, 5, 7, 8, or 10, or the nucleotide sequence of the DNA insert of the plasmid deposited with ATCC as Accession Number ______, ______, or ______, due to degeneracy of the genetic code and thus encode the same HEAT proteins as those encoded by the nucleotide sequence shown in SEQ ID NO:1, 3, 5, 7, 8, or 10, or the nucleotide sequence of the DNA insert of the plasmid deposited with ATCC as Accession Number ______, ______, or ______. In another embodiment, an isolated nucleic acid molecule of the invention has a nucleotide sequence encoding a protein having an amino acid sequence which differs by at least 1, but no greater than 5, 10, 20, 50 or 100 amino acid residues from the amino acid sequence shown in SEQ ID NO:2, 6, or 9, or the amino acid sequence encoded by the DNA insert of the plasmid deposited with the ATCC as Accession Number ______, ______, or ______. In yet another embodiment, the nucleic acid molecule encodes the amino acid sequence of human HEAT. If an alignment is needed for this comparison, the sequences should be aligned for maximum homology.

Nucleic acid variants can be naturally occurring, such as allelic variants (same locus), homologues (different locus), and orthologues (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

Allelic variants result, for example, from DNA sequence polymorphisms within a population (e.g., the human population) that lead to changes in the amino acid sequences of the HEAT proteins. Such genetic polymorphism in the HEAT genes may exist among individuals within a population due to natural allelic variation. As used herein, the terms “gene” and “recombinant gene” refer to nucleic acid molecules which include an open reading frame encoding a HEAT protein, preferably a mammalian HEAT protein, and can further include non-coding regulatory sequences, and introns.

Accordingly, in one embodiment, the invention features isolated nucleic acid molecules which encode a naturally occurring allelic variant of a polypeptide comprising the amino acid sequence of SEQ ID NO:2, 6, or 9, or an amino acid sequence encoded by the DNA insert of the plasmid deposited with ATCC as Accession Number ______, ______, or ______, wherein the nucleic acid molecule hybridizes to a complement of a nucleic acid molecule comprising SEQ ID NO:1, 3, 5, 7, 8, or 10, for example, under stringent hybridization conditions.

Allelic variants of HEAT, e.g., human HEAT-1, HEAT-2, or HEAT-3, include both functional and non-functional HEAT proteins. Functional allelic variants are naturally occurring amino acid sequence variants of the HEAT protein that maintain the ability to, e.g., bind or interact with a HEAT substrate or target molecule, transport a HEAT substrate or target molecule across a biological membrane, hydrolyze ATP, be phosphorylated or dephosphorylated, adopt an E1 conformation or an E2 conformation, and/or modulate cellular signaling, vascular smooth muscle tone, cellular growth and/or proliferation, and/or angiogenesis. Functional allelic variants will typically contain only a conservative substitution of one or more amino acids of SEQ ID NO:2, 6, or 9, or a substitution, deletion or insertion of non-critical residues in non-critical regions of the protein.

Non-functional allelic variants are naturally occurring amino acid sequence variants of the HEAT protein. e.g, human HEAT-1, HEAT-2, or HEAT-3, that do not have the ability to, e.g., bind or interact with a HEAT substrate or target molecule, transport a HEAT substrate or target molecule across a biological membrane, hydrolyze ATP, be phosphorylated or dephosphorylated, adopt an E1 conformation or an E2 conformation, and/or modulate cellular signaling, vascular smooth muscle tone, cellular growth and/or proliferation, and/or angiogenesis. Non-functional allelic variants will typically contain a non-conservative substitution, a deletion, or insertion, or premature truncation of the amino acid sequence of SEQ ID NO:2, 6, or 9, or a substitution, insertion, or deletion in critical residues or critical regions of the protein.

The present invention further provides non-human orthologues (e.g., non-human orthologues of the human HEAT-1, HEAT-2, or HEAT-3 proteins). Orthologues of the human HEAT proteins are proteins that are isolated from non-human organisms and possess the same HEAT substrate or target molecule binding mechanisms, Ca2⁺ transporting activity, ATPase activity, and/or modulation of cellular signaling mechanisms of the human HEAT proteins. Orthologues of the human HEAT proteins can readily be identified as comprising an amino acid sequence that is substantially homologous to SEQ ID NO:2, 6, or 9.

Moreover, nucleic acid molecules encoding other HEAT family members and, thus, which have a nucleotide sequence which differs from the HEAT sequences of SEQ ID NO:1, 3, 5, 7, 8, or 10, or the nucleotide sequence of the DNA insert of the plasmid deposited with ATCC as Accession Number ______, ______, or ______ are intended to be within the scope of the invention. For example, another HEAT cDNA can be identified based on the nucleotide sequence of human HEAT-1, HEAT-2, or HEAT-3. Moreover, nucleic acid molecules encoding HEAT proteins from different species, and which, thus, have a nucleotide sequence which differs from the HEAT sequences of SEQ ID NO:1, 3, 5, 7, 8, or 10, or the nucleotide sequence of the DNA insert of the plasmid deposited with ATCC as Accession Number ______, ______, or ______ are intended to be within the scope of the invention. For example, a mouse or monkey HEAT cDNA can be identified based on the nucleocide sequence of a human HEAT-1, HEAT-2, or HEAT-3.

Nucleic acid molecules corresponding to natural allelic variants and homologues of the HEAT cDNAs of the invention can be isolated based on their homology to the HEAT nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the HEAT cDNAs of the invention can further be isolated by mapping to the same chromosome or locus as the HEAT gene.

Orthologues, homologues, and allelic variants can be identified using methods known in the art (e.g., by hybridization to an isolated nucleic acid molecule of the present invention, for example, under stringent hybridization conditions). In one embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:1, 3, 5, 7, 8, or 10, or the nucleotide sequence of the DNA insert of the plasmid deposited with ATCC as Accession Number ______, ______, or ______. In other embodiment, the nucleic acid is at least 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1250, 1300, 1350, 1400, 1450, 1500, 1550, 1600, 1650, 1700, 1750, 1800, 1850, 1900, 1950, 1994, 2000, 2050, 2073, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3441, 3450, 3500, 3550, 3600, 3650, 3700, 3750, 3800, 3841, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 5000, 5050, 5100, 5150, 5200, 5250, 5300, 5350, 5400, 5450, 5500, 5550, 5600, 5650, 5700, 5750, 5800, 5850, 5900, 5950, 6000, 6050, 6100, 6150, 6200, 6250, 6300, 6350, 6400, 6450, 6500, 6550, 6600, 6650, 6700, 6750, 6800, 6850, 6900, 6950, 7000, 7050, 7100, 7150, 7200 or more nucleotides in length.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4, and 6. Additional stringent conditions can be found in Molecular Cloning: A Laboratory Manual, Sambrook et al., Cold Spring Harbor Press, Cold Spring Harbor, N.Y. (1989), chapters 7, 9, and 11. A preferred, non-limiting example of stringent hybridization conditions includes hybridization in 4×sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)-(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (see e.g., Church and Gilbert (1984) Proc. Natl. Acad. Sci. USA 81:1991-1995), or alternatively 0.2×SSC, 1% SDS.

Preferably, an isolated nucleic acid molecule of the invention that hybridizes under stringent conditions to the sequence of SEQ ID NO:1, 3, 5, 7, 8, or 10 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the HEAT sequences that may exist in the population, the skilled artisan will further appreciate that changes can be introduced by mutation into the nucleotide sequences of SEQ ID NO:1, 3, 5, 7, 8, or 10, or the nucleotide sequence of the DNA insert of the plasmid deposited with ATCC as Accession Number ______, ______, or ______, thereby leading to changes in the amino acid sequence of the encoded HEAT proteins, without altering the functional ability of the HEAT proteins. For example, nucleotide substitutions leading to amino acid substitutions at “non-essential” amino acid residues can be made in the sequence of SEQ ID NO:1, 3, 5, 7, 8, or 10, or the nucleotide sequence of the DNA insert of the plasmid deposited with ATCC as Accession Number ______, ______, or ______. A “non-essential” amino acid residue is a residue that can be altered from the wild-type sequence of HEAT-2 or HEAT-3 (e.g., the sequence of SEQ ID NO:2, 6, or 9) without altering the biological activity, whereas an “essential” amino acid residue is required for biological activity. For example, amino acid residues that are conserved among the HEAT proteins of the present invention, e.g., those present in an E1-E2 ATPase domain or an E1-E2 ATPases phosphorylation site, are predicted to be particularly unamenable to alteration. Furthermore, additional amino acid residues that are conserved between the HEAT proteins of the present invention and other members of the E1-E2 ATPase family are not likely to be amenable to alteration.

Accordingly, another aspect of the invention pertains to nucleic acid molecules encoding HEAT proteins that contain changes in amino acid residues that are not essential for activity. Such HEAT proteins differ in amino acid sequence from SEQ ID NO:2, 6, or 9, yet retain biological activity. In one embodiment, the isolated nucleic acid molecule comprises a nucleotide sequence encoding a protein, wherein the protein comprises an amino acid sequence at least about 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more homologous to SEQ ID NO:2, 6, or 9, e.g., to the entire length of SEQ ID NO:2, 6, or 9.

An isolated nucleic acid molecule encoding a HEAT protein homologous to the protein of SEQ ID NO:2, 6, or 9 can be created by introducing one or more nucleotide substitutions, additions or deletions into the nucleotide sequence of SEQ ID NO:1, 3, 5, 7, 8, or 10, or the nucleotide sequence of the DNA insert of the plasmid deposited with ATCC as Accession Number ______, ______, or ______, such that one or more amino acid substitutions, additions or deletions are introduced into the encoded protein. Mutations can be introduced into SEQ ID NO:1, 3, 5, 7, 8, or 10, or the nucleotide sequence of the DNA insert of the plasmid deposited with ATCC as Accession Number ______, _______, or ______ by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in a HEAT protein is preferably replaced with another amino acid residue from the same side chain family. Alternatively, in another embodiment, mutations can be introduced randomly along all or part of a HEAT coding sequence, such as by saturation mutagenesis, and the resultant mutants can be screened for HEAT biological activity to identify mutants that retain activity. Following mutagenesis of SEQ ID NO:1, 3, 5, 7, 8, or 10, or the nucleotide sequence of the DNA insert of the plasmid deposited with ATCC as Accession Number ______, ______, or ______, the encoded protein can be expressed recombinantly and the activity of the protein can be determined.

In a preferred embodiment, a mutant HEAT protein can be assayed for the ability to (i) interact with a HEAT substrate or target molecule (e.g., a Ca²⁺ ion; ATP; or a non-HEAT protein;); (ii) transport of HEAT substrate or target molecule (e.g., a Ca²⁺ ion) from one side of a biological membrane to the other; (iii) be phosphorylated or dephosphorylated; (iv) adopt an E1 conformation or an E2 conformation; (v) convert a HEAT substrate or target molecule to a product (e.g., hydrolyze ATP to ADP and free phosphate); (vi) interact with a second non-HEAT protein; (vii) modulate intra- or inter-cellular signaling and/or gene transcription (e.g., either directly or indirectly); (viii) modulate vascular smooth muscle tone; (ix) modulate cellular growth and/or proliferation; and/or (x) modulate angiogenesis.

In addition to the nucleic acid molecules encoding HEAT proteins described above, another aspect of the invention pertains to isolated nucleic acid molecules which are antisense thereto. In an exemplary embodiment, the invention provides an isolated nucleic acid molecule which is antisense to a HEAT nucleic acid molecule (e.g., is antisense to the coding strand of a HEAT nucleic acid molecule). An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire HEAT coding strand, or to only a portion thereof. In one embodiment, an antisense nucleic acid molecule is antisense to “coding region sequences” of the coding strand of a nucleotide sequence encoding HEAT-1, HEAT-2, or HEAT-3. The term “coding region sequences” refers to the region of the nucleotide sequence comprising codons which are translated into amino acid residues (e.g., the coding region sequences of human HEAT-1, HEAT-2, or HEAT-3, corresponding to SEQ ID NO:3, 7, or 10, respectively). In another embodiment, the antisense nucleic acid molecule is antisense to a “noncoding region” of the coding strand of a nucleotide sequence encoding HEAT-1, HEAT-2, or HEAT-3. The term “noncoding region” refers to 5′ (e.g., nucleotides 1-209 of SEQ ID NO:1 (HEAT-1), nucleotides 1-224 of SEQ ID NO:5 (HEAT-2), or nucleotides 1-67 of SEQ ID NO:8 (HEAT-3)) and/or 3′ sequences (e.g., nucleotides 3750-4055 of SEQ ID NO:1 (HEAT-1), nucleotides 3993-7249 of SEQ ID NO:5 (HEAT-2) or nucleotides 3680-3919 of SEQ ID NO:8 (HEAT-3)) which flank the coding region sequences that are not translated into amino acids (also referred to as 5′ and 3′ untranslated regions).

Given the coding strand sequences encoding HEAT-1, HEAT-2, and HEAT-3 disclosed herein (e.g., SEQ ID NOs:3, 7, and 10, respectively), antisense nucleic acids of the invention can be designed according to the rules of Watson and Crick base pairing. The antisense nucleic acid molecule can be complementary to coding region sequences of HEAT-1, HEAT-2, or HEAT-3 mRNA, but more preferably is an oligonucleotide which is antisense to only a portion of the HEAT-1, HEAT-2, or HEAT-3 mRNA. An antisense oligonucleotide can be, for example, about 5, 10, 15, 20, 25, 30, 35, 40, 45, 50, 55, 60, 65, 70, 75, 80, or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5 -methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a HEAT protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave HEAT mRNA transcripts to thereby inhibit translation of HEAT mRNA. A ribozyme having specificity for a HEAT-encoding nucleic acid can be designed based upon the nucleotide sequence of a HEAT cDNA disclosed herein (i.e., SEQ ID NO:1, 3, 5, 7, 8, or 10, or the nucleotide sequence of the DNA insert of the plasmid deposited with ATCC as Accession Number ______, ______, or ______). For example, a derivative of a Tetrahymena L-19 IVS RNA can be constructed in which the nucleotide sequence of the active site is complementary to the nucleotide sequence to be cleaved in a HEAT-encoding mRNA. See, e.g., Cech et al., U.S. Pat. No. 4,987,071; and Cech et al., U.S. Pat. No. 5,116,742. Alternatively, HEAT mRNA can be used to select a catalytic RNA having a specific ribonuclease activity from a pool of RNA molecules. See, e.g., Bartel, D. and Szostak, J. W. (1993) Science 261:1411-1418.

Alternatively, HEAT gene expression can be inhibited by targeting nucleotide sequences complementary to the regulatory region of the HEAT gene (e.g., the HEAT-1 promoter and/or enhancers; the HEAT-2 promoter and/or enhancers; or the HEAT-3 promoter and/or enhancers) to form triple helical structures that prevent transcription of the HEAT gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioessays 14(12):807-15.

In yet another embodiment, the HEAT nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup, B. and Nielsen, P. E. (1996) Bioorg. Med. Chem. 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup and Nielsen (1996) supra and Perry-O'Keefe et al. (1996) Proc. Natl. Acad. Sci. USA 93:14670-675.

PNAs of HEAT nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of HEAT nucleic acid molecules can also be used in the analysis of single base pair mutations in a gene (e.g., by PNA-directed PCR clamping); as 'artificial restriction enzymes' when used in combination with other enzymes (e.g., S1 nucleases (Hyrup and Nielsein (1996) supra)); or as probes or primers for DNA sequencing or hybridization (Hyrup and Nielsen (1996) supra; Perry-O'Keefe et al. (1996) supra).

In another embodiment, PNAs of HEAT can be modified (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of HEAT nucleic acid molecules can be generated which may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup and Nielsen (1996) supra). The synthesis of PNA-DNA chimeras can be performed as described in Hyrup and Nielsen (1996) supra and Finn, P. J. et al. (1996) Nucleic Acids Res. 24(17):3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acids Res. 17:5973-88). PNA monomers are then coupled in a stepwise manner to produce a chimeric molecule with a 5′ PNA segment and a 3′ DNA segment (Finn, P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (See, e.g., Krol et al. (1988) Biotechniques 6:958-976) or intercalating agents (See, e.g., Zon (1988) Pharm. Res. 5:539-549). To this end, the oligonucleotide may be conjugated to another molecule (e.g., a peptide, hybridization triggered cross-linking agent, transport agent, or hybridization-triggered cleavage agent).

II. Isolated HEAT Proteins and Anti-HEAT Antibodies

One aspect of the invention pertains to isolated or recombinant HEAT proteins and polypeptides, and biologically active portions thereof, as well as polypeptide fragments suitable for use as immunogens to raise anti-HEAT antibodies. In one embodiment, native HEAT proteins can be isolated from cells or tissue sources by an appropriate purification scheme using standard protein purification techniques. In another embodiment, HEAT proteins are produced by recombinant DNA techniques. Alternative to recombinant expression, a HEAT protein or polypeptide can be synthesized chemically using standard peptide synthesis techniques.

An “isolated” or “purified” protein or biologically active portion thereof is substantially free of cellular material or other contaminating proteins from the cell or tissue source from which the HEAT protein is derived, or substantially free from chemical precursors or other chemicals when chemically synthesized. The language “substantially free of cellular material” includes preparations of HEAT protein in which the protein is separated from cellular components of the cells from which it is isolated or recombinantly produced. In one embodiment, the language “substantially free of cellular material” includes preparations of HEAT protein having less than about 30% (by dry weight) of non-HEAT protein (also referred to herein as a “contaminating protein”), more preferably less than about 20% of non-HEAT protein, still more preferably less than about 10% of non-HEAT protein, and most preferably less than about 5% non-HEAT protein. When the HEAT protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of HEAT protein in which the protein is separated from chemical precursors or other chemicals which are involved in the synthesis of the protein. In one embodiment, the language “substantially free of chemical precursors or other chemicals” includes preparations of HEAT protein having less than about 30% (by dry weight) of chemical precursors or non-HEAT chemicals, more preferably less than about 20% chemical precursors or non-HEAT chemicals, still more preferably less than about 10% chemical precursors or non-HEAT chemicals, and most preferably less than about 5% chemical precursors or non-HEAT chemicals.

As used herein, a “biologically active portion” of a HEAT protein includes a fragment of a HEAT protein which participates in an interaction between a HEAT molecule and a non-HEAT molecule (e.g., a HEAT substrate such as Ca²⁺, ATP or a non-HEAT protein). Biologically active portions of a HEAT protein include peptides comprising amino acid sequences sufficiently homologous to or derived from the HEAT amino acid sequences, e.g., the amino acid sequences shown in SEQ ID NO:2, 6, or 9, which include sufficient amino acid residues to exhibit at least one activity of a HEAT protein. Typically, biologically active portions comprise a domain or motif with at least one activity of the HEAT protein, e.g., the ability to interact with a HEAT substrate or target molecule (e.g., a Ca²⁺ ion; ATP; or a non-HEAT protein;); the ability to transport a HEAT substrate or target molecule (e.g., a Ca²⁺ ion) from one side of a biological membrane to the other; the ability to be phosphorylated or dephosphorylated; the ability to adopt an E1 conformation or an E2 conformation; the ability to convert a HEAT substrate or target molecule to a product (e.g., the ability to hydrolyze ATP to ADP and free phosphate); the ability to interact with a second non-HEAT protein; the ability to modulate intra- or inter-cellular signaling and/or gene transcription (e.g., either directly or indirectly); the ability to modulate vascular smooth muscle tone; the ability to modulate cellular growth and/or proliferation; and/or the ability to modulate angiogenesis. A biologically active portion of a HEAT protein can be a polypeptide which is, for example, 10, 25, 50, 75, 100, 125, 150, 175, 200, 250, 300, 350, 400, 450, 500, 550, 600, 650, 691, 700, 750, 800, 850, 900, 950, 1000, 1100, 1050, 1060, 1072, 1100, 1150, 1200, 1250 or more amino acids in length. Biologically active portions of a HEAT protein can be used as targets for developing agents which modulate a HEAT mediated activity, e.g., any of the aforementioned HEAT activities.

In one embodiment, a biologically active portion of a HEAT protein comprises at least at least one or more of the following domains or motifs: a transmembrane domain, an E1-E2 ATPase domain, an E1-E2 ATPases phosphorylation site, an N-terminal large extramembrane domain, a C-terminal large extramembrane domain, a P-type ATPase sequence 1 motif, a P-type ATPase sequence 2 motif, and/or a P-type ATPase sequence 3 motif. Moreover, other biologically active portions, in which other regions of the protein are deleted, can be prepared by recombinant techniques and evaluated for one or more of the functional activities of a native HEAT protein.

Another aspect of the invention features fragments of the protein having the amino acid sequence of SEQ ID NO:2, 6, or 9, for example, for use as immunogens. In one embodiment, a fragment comprises at least 8 amino acids (e.g., contiguous or consecutive amino acids) of the amino acid sequence of SEQ ID NO:2, 6, or 9, or an amino acid sequence encoded by the DNA insert of the plasmid deposited with the ATCC as Accession Number ______, ______, or ______. In another embodiment, a fragment comprises at least 8, 10, 15, 20, 25, 30, 35, 40, 45, 50 or more amino acids (e.g., contiguous or consecutive amino acids) of the amino acid sequence of SEQ ID NO:2, 6, or 9, or an amino acid sequence encoded by the DNA insert of the plasmid deposited with the ATCC as Accession Number ______, ______, or ______.

In a preferred embodiment, a HEAT protein has an amino acid sequence shown in SEQ ID NO:2, 6, or 9. In other embodiments, the HEAT protein is substantially identical to SEQ ID NO:2, 6, or 9, and retains the functional activity of the protein of SEQ ID NO:2, 6, or 9, yet differs in amino acid sequence due to natural allelic variation or mutagenesis, as described in detail in subsection I above. In another embodiment, the HEAT protein is a protein which comprises an amino acid sequence at least about 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to SEQ ID NO:2, 6, or 9.

In another embodiment, the invention features a HEAT protein which is encoded by a nucleic acid molecule consisting of a nucleotide sequence at least about 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to a nucleotide sequence of SEQ ID NO:1, 3, 5, 7, 8, or 10, or a complement thereof. This invention further features a HEAT protein which is encoded by a nucleic acid molecule consisting of a nucleotide sequence which hybridizes under stringent hybridization conditions to a complement of a nucleic acid molecule comprising the nucleotide sequence of SEQ ID NO:1, 3, 5, 7, 8, or 10, or a complement thereof.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the HEAT-1 amino acid sequence of SEQ ID NO:2 having 1180 amino acid residues, at least 354, preferably at least 472, more preferably at least 590, even more preferably at least 708, and even more preferably at least 826, 944, or 1062 amino acid residues are aligned; when aligning a second sequence to the HEAT-2 amino acid sequence of SEQ ID NO:6 having 1256 amino acid residues, at least 377, preferably at least 502, more preferably at least 628, even more preferably at least 755, and even more preferably at least 879, 1005 or 1130 amino acid residues are aligned; when aligning a second sequence to the HEAT-3 amino acid sequence of SEQ ID NO:9 having 1204 amino acid residues, at least 361, preferably at least 482, more preferably at least 602, even more preferably at least 722, and even more preferably at least 844, 963 or 1084 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at online through the Genetics Computer Group), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at online through the Genetics Computer Group), using a NWSgapdna.CMP matrix and a gap weight of 40, 50, 60, 70, or 80 and a length weight of 1, 2, 3, 4, 5, or 6. A preferred, non-limiting example of parameters to be used in conjunction with the GAP program include a Blosum 62 scoring matrix with a gap penalty of 12, a gap extend penalty of 4, and a frameshift gap penalty of 5.

In another embodiment, the percent identity between two amino acid or nucleotide sequences is determined using the algorithm of Meyers and Miller (Comput. Appl. Biosci. 4:11-17 (1988)) which has been incorporated into the ALIGN program (version 2.0 or version 2.0U), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to HEAT nucleic acid molecules of the invention. BLAST protein searches can be performed with the XBLAST program, score=50, wordlength=3 to obtain amino acid sequences homologous to HEAT protein molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g, XBLAST and NBLAST) can be used. See the website for the National Center for Biotechnology Information.

The invention also provides HEAT chimeric or fusion proteins. As used herein, a HEAT “chimeric protein” or “fusion protein” comprises a HEAT polypeptide operatively linked to a non-HEAT polypeptide. A “HEAT polypeptide” refers to a polypeptide having an amino acid sequence corresponding to HEAT-1, HEAT-2, or HEAT-3, for example, whereas a “non-HEAT polypeptide” refers to a polypeptide having an amino acid sequence corresponding to a protein which is not substantially homologous to the HEAT protein, e.g, a protein which is different from the HEAT protein and which is derived from the same or a different organism. Within a HEAT fusion protein the HEAT polypeptide can correspond to all or a portion of a HEAT protein. In a preferred embodiment, a HEAT fusion protein comprises at least one biologically active portion of a HEAT protein. In another preferred embodiment, a HEAT fusion protein comprises at least two biologically active portions of a HEAT protein. Within the fusion protein, the term “operatively linked” is intended to indicate that the HEAT polypeptide and the non-HEAT polypeptide are fused in-frame to each other. The non-HEAT polypeptide can be fused to the N-terminus or C-terminus of the HEAT polypeptide.

For example, in one embodiment, the fusion protein is a GST-HEAT fusion protein in which the HEAT sequences are fused to the C-terminus of the GST sequences. Such fusion proteins can facilitate the purification of recombinant HEAT. In another embodiment, the fusion protein is a HEAT protein containing a heterologous signal sequence at its N-terminus. In certain host cells (e.g., mammalian host cells), expression and/or secretion of HEAT can be increased through use of a heterologous signal sequence.

In another embodiment, a HEAT “chimeric protein” or “fusion protein” comprises a HEAT protein of the present invention wherein one or more domains or motifs in the HEAT protein are replaced with the corresponding domains or motifs from another HEAT protein or another E1-E1 or P-type ATPase. Such HEAT chimeric proteins are useful, for example, for determining how individual motifs or domains contribute to or influence the activity of a HEAT protein. Such HEAT chimeric proteins are also useful, for example, in creating HEAT proteins with certain activities of one HEAT protein and certain activities of a different HEAT protein, or with certain activities of a HEAT protein and certain activities of a different E1-E2 or P-type ATPase.

The HEAT fusion proteins of the invention can be incorporated into pharmaceutical compositions and administered to a subject in vivo. The HEAT fusion proteins can be used to affect the bioavailability of a HEAT substrate. Use of HEAT fusion proteins may be useful therapeutically for the treatment of disorders caused by, for example, (i) aberrant modification or mutation of a gene encoding a HEAT protein; (ii) mis-regulation of the HEAT gene; and (iii) aberrant post-translational modification of a HEAT protein.

Moreover, the HEAT-fusion proteins of the invention can be used as immunogens to produce anti-HEAT antibodies in a subject, to purify HEAT substrates, and in screening assays to identify molecules which inhibit or enhance the interaction of HEAT with a HEAT substrate.

Preferably, a HEAT chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al., John Wiley & Sons:1992). Moreover, many expression vectors are commercially available that already encode a fusion moiety (e.g., a GST polypeptide). A HEAT-encoding nucleic acid can be cloned into such an expression vector such that the fusion moiety is linked in-frame to the HEAT protein.

The present invention also pertains to variants of the HEAT proteins which function as either HEAT agonists (mimetics) or as HEAT antagonists. Variants of the HEAT proteins can be generated by mutagenesis, e.g., discrete point mutation or truncation of a HEAT protein. An agonist of the HEAT proteins can retain substantially the same, or a subset, of the biological activities of the naturally occurring form of a HEAT protein. An antagonist of a HEAT protein can inhibit one or more of the activities of the naturally occurring form of the HEAT protein by, for example, competitively modulating a HEAT-mediated activity of a HEAT protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the HEAT protein.

In one embodiment, variants of a HEAT protein which function as either HEAT agonists (mimetics) or as HEAT antagonists can be identified by screening combinatorial libraries of mutants, e.g., truncation mutants, of a HEAT protein for HEAT protein agonist or antagonist activity. In one embodiment, a variegated library of HEAT variants is generated by combinatorial mutagenesis at the nucleic acid level and is encoded by a variegated gene library. A variegated library of HEAT variants can be produced by, for example, enzymatically ligating a mixture of synthetic oligonucleotides into gene sequences such that a degenerate set of potential HEAT sequences is expressible as individual polypeptides, or alternatively, as a set of larger fusion proteins (e.g., for phage display) containing the set of HEAT sequences therein. There are a variety of methods which can be used to produce libraries of potential HEAT variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential HEAT sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acids Res. 11:477.

In addition, libraries of fragments of a HEAT protein coding sequence can be used to generate a variegated population of HEAT fragments for screening and subsequent selection of variants of a HEAT protein. In one embodiment, a library of coding sequence fragments can be generated by treating a double stranded PCR fragment of a HEAT coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the HEAT protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of HEAT proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify HEAT variants (Arkin and Youvan (1992) Proc. Natl. Acad. Sci. USA 89:7811-7815; Delagrave et al. (1993) Protein Eng. 6(3):327-331).

In one embodiment, cell based assays can be exploited to analyze a variegated HEAT library. For example, a library of expression vectors can be transfected into a cell line which ordinarily responds to HEAT in a particular HEAT substrate-dependent manner. The transfected cells are then contacted with HEAT and the effect of the expression of the mutant on signaling by the HEAT substrate can be detected by measuring e.g., Ca²⁺ transport (e.g., by measuring Ca²⁺ levels inside the cell or its various cellular compartments, or in the extracellular medium), hydrolysis of ATP, phosphorylation or dephosphorylation of the HEAT protein, and/or gene transcription. Plasmid DNA can then be recovered from the cells which score for inhibition, or alternatively, potentiation of signaling by the HEAT substrate, or which score for increased or decreased levels of Ca²⁺ transport or ATP hydrolysis, and the individual clones further characterized.

An isolated HEAT protein, or a portion or fragment thereof, can be used as an immunogen to generate antibodies that bind HEAT using standard techniques for polyclonal and monoclonal antibody preparation. A full-length HEAT protein can be used or, alternatively, the invention provides antigenic peptide fragments of HEAT for use as immunogens. The antigenic peptide of HEAT comprises at least 8 amino acid residues of the amino acid sequence shown in SEQ ID NO:2, 6, or 9 and encompasses an epitope of HEAT such that an antibody raised against the peptide forms a specific immune complex with HEAT. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of HEAT that are located on the surface of the protein, e.g. hydrophilic regions, as well as regions with high antigenicity (see, for example, FIGS. 2, 13, and 24).

A HEAT immunogen typically is used to prepare antibodies by immunizing a suitable subject (e.g., rabbit, goat, mouse, or other mammal) with the immunogen. An appropriate immunogenic preparation can contain, for example, recombinantly expressed HEAT protein or a chemically-synthesized HEAT polypeptide. The preparation can further include an adjuvant, such as Freund's complete or incomplete adjuvant, or similar immunostimulatory agent. Immunization of a suitable subject with an immunogenic HEAT preparation induces a polyclonal anti-HEAT antibody response.

Accordingly, another aspect of the invention pertains to anti-HEAT antibodies. The term “antibody” as used herein refers to immunoglobulin molecules and immunologically active portions of immunoglobulin molecules, i.e., molecules that contain an antigen binding site which specifically binds (immunoreacts with) an antigen, such as HEAT. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind HEAT. The term “monoclonal antibody” or “monoclonal antibody composition”, as used herein, refers to a population of antibody molecules that contain only one species of an antigen binding site capable of immunoreacting with a particular epitope of HEAT. A monoclonal antibody composition thus typically displays a single binding affinity for a particular HEAT protein with which it immunoreacts.

Polyclonal anti-HEAT antibodies can be prepared as described above by immunizing a suitable subject with a HEAT immunogen. The anti-HEAT antibody titer in the immunized subject can be monitored over time by standard techniques, such as with an enzyme linked immunosorbent assay (ELISA) using immobilized HEAT. If desired, the antibody molecules directed against HEAT can be isolated from the mammal (e.g., from the blood) and further purified by well known techniques, such as protein A chromatography to obtain the IgG fraction. At an appropriate time after immunization, e.g., when the anti-HEAT antibody titers are highest, antibody-producing cells can be obtained from the subject and used to prepare monoclonal antibodies by standard techniques, such as the hybridoma technique originally described by Kohler and Milstein (1975) Nature 256:495-497 (see also Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally Kenneth, R. H., in Monoclonal Antibodies: A New Dimension In Biological Analyses, Plenum Publishing Corp., New York, N.Y. (1980); Lerner, E. A. (1981) Yale J. Biol. Med, 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with a HEAT immunogen as described above, and the culture supernatants of the resulting hybridoma cells are screened to identify a hybridoma producing a monoclonal antibody that binds HEAT.

Any of the many well known protocols used for fusing lymphocytes and immortalized cell lines can be applied for the purpose of generating an anti-HEAT monoclonal antibody (see, e.g., Galfre, G. et al. (1977) Nature 266:55052; Gefter et al. (1977) supra; Lerner (1981) supra; Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63- Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind HEAT, e.g., using a standard ELISA assay.

Alternative to preparing monoclonal antibody-secreting hybridomas, a monoclonal anti-HEAT antibody can be identified and isolated by screening a recombinant combinatorial immunoglobulin library (e.g., an antibody phage display library) with HEAT to thereby isolate immunoglobulin library members that bind HEAT. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al., U.S. Pat. No. 5,223,409; Kang et al., PCT International Publication No. WO 92/18619; Dower et al., PCT International Publication No. WO 91/17271; Winter et al., PCT International Publication No. WO 92/20791; Markland et al. PCT International Publication No. WO 92/15679; Breitling et al., PCT International Publication No. WO 93/01288; McCafferty et al., PCT International Publication No. WO 92/01047; Garrard et al., PCT International Publication No. WO 92/09690; Ladner et al., PCT International Publication No. WO 90/02809; Fuchs et al. (1991) Biotechnology (NY) 9:1369-1372; Hay et al. (1992) Hum. Antibodies Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Biotechnology (N.Y.) 9:1373-1377; Hogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant anti-HEAT antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al., International Application No. PCT/US86/02269; Akira et al., European Patent Application No. 184,187; Taniguchi, M., European Patent Application No. 171,496; Morrison et al., European Patent Application 173,494; Neuberger et al., PCT International Publication No. WO 86/01533; Cabilly et al., U.S. Pat. No. 4,816,567; Cabilly et al., European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter, U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-HEAT antibody (e.g., monoclonal antibody) can be used to isolate HEAT by standard techniques, such as affinity chromatography or immunoprecipitation. An anti-HEAT antibody can facilitate the purification of natural HEAT from cells and of recombinantly produced HEAT expressed in host cells. Moreover, an anti-HEAT antibody can be used to detect HEAT protein (e.g., in a cellular lysate or cell supernatant) in order to evaluate the abundance and pattern of expression of the HEAT protein. Anti-HEAT antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, for example recombinant expression vectors, containing a HEAT nucleic acid molecule or vectors containing a nucleic acid molecule which encodes a HEAT protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g. polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., HEAT proteins, mutant forms of HEAT proteins, fusion proteins, and the like).

Accordingly, an exemplary embodiment provides a method for producing a protein, preferably a HEAT protein, by culturing in a suitable medium a host cell of the invention (e.g., a mammalian host cell such as a non-human mammalian cell) containing a recombinant expression vector, such that the protein is produced.

The recombinant expression vectors of the invention can be designed for expression of HEAT proteins in prokaryotic or eukaryotic cells. For example, HEAT proteins can be expressed in bacterial cells such as E. coli, insect cells (using baculovirus expression vectors) yeast cells or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in HEAT activity assays (e.g., direct assays or competitive assays described in detail below), or to generate antibodies specific for HEAT proteins, for example. In a preferred embodiment, a HEAT fusion protein expressed in a retroviral expression vector of the present invention can be utilized to infect bone marrow cells, which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al. (1988) Gene 69:301-315) and pET 11d (Studier et al. (1990) Methods Enzymol. 185:60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S. (1990) Methods Enzymol. 185:119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the HEAT expression vector is a yeast expression vector. Examples of vectors for expression in yeast S. cerevisiae include pYepSec1 (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and picZ (Invitrogen Corp., San Diego, Calif.).

Alternatively, HEAT proteins can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2^{nd} ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to HEAT mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al. “Antisense RNA as a molecular tool for genetic analysis”, Reviews—Trends in Genetics, Vol. 1(1) 1986.

Another aspect of the invention pertains to host cells into which a HEAT nucleic acid molecule of the invention is introduced, e.g., a HEAT nucleic acid molecule within a vector (e.g., a recombinant expression vector) or a HEAT nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a HEAT protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2^{nd} ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify an d select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a HEAT protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a HEAT protein. Accordingly, the invention further provides methods for producing a HEAT protein using the host cells of the invention. In one embodiment, the method comprises culturing the host cell of the invention (into which a recombinant expression vector encoding a HEAT protein has been introduced) in a suitable medium such that a HEAT protein is produced. In another embodiment, the method further comprises isolating a HEAT protein from the medium or the host cell.

The host cells of the invention can also be used to produce non-human transgenic animals. For example, in one embodiment, a host cell of the invention is a fertilized oocyte or an embryonic stem cell into which HEAT coding sequences have been introduced. Such host cells can then be used to create non-human transgenic animals in which exogenous HEAT sequences have been introduced into their genome or homologous recombinant animals in which endogenous HEAT sequences have been altered. Such animals are useful for studying the function and/or activity of a HEAT protein and for identifying and/or evaluating modulators of HEAT activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous HEAT gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a HEAT-encoding nucleic acid into the male pronuclei of a fertilized oocyte, e.g., by microinjection or retroviral infection, and allowing the oocyte to develop in a pseudopregnant female foster animal. The HEAT cDNA sequence of SEQ ID NO:1, 5, or 8 can be introduced as a transgene into the genome of a non-human animal. Alternatively, a non-human homologue of a human HEAT gene, such as a rat or mouse HEAT gene, can be used as a transgene. Alternatively, a HEAT gene homologue, such as another HEAT family member, can be isolated based on hybridization to the HEAT cDNA sequences of SEQ ID NO:1, 3, 5, 7, 8, or 10, or the DNA insert of the plasmid deposited with ATCC as Accession Number _____,_ ______, or ______ (described further in subsection I above) and used as a transgene. Intronic sequences and polyadenylation signals can also be included in the transgene to increase the efficiency of expression of the transgene. A tissue-specific regulatory sequence(s) can be operably linked to a HEAT transgene to direct expression of a HEAT protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of a HEAT transgene in its genome and/or expression of HEAT mRNA in tissues or cells of the animals. A transgenic founder animal can then be used to breed additional animals carrying the transgene. Moreover, transgenic animals carrying a transgene encoding a HEAT protein can further be bred to other transgenic animals carrying other transgenes.

To create a homologous recombinant animal, a vector is prepared which contains at least a portion of a HEAT gene into which a deletion, addition or substitution has been introduced to thereby alter, e.g., functionally disrupt, the HEAT gene. The HEAT gene can be a human gene (e.g., the cDNA of SEQ ID NO:3, 7, or 10), but more preferably, is a non-human homologue of a human HEAT gene (e.g., a cDNA isolated by stringent hybridization with the nucleotide sequence of SEQ ID NO:1, 5, or 8), For example, a mouse HEAT gene can be used to construct a homologous recombination nucleic acid molecule, e.g., a vector, suitable for altering an endogenous HEAT gene in the mouse genome. In a preferred embodiment, the homologous recombination nucleic acid molecule is designed such that, upon homologous recombination, the endogenous HEAT gene is functionally disrupted (i.e., no longer encodes a functional protein; also referred to as a “knock out” vector). Alternatively, the homologous recombination nucleic acid molecule can be designed such that, upon homologous recombination, the endogenous HEAT gene is mutated or otherwise altered but still encodes functional protein (e.g., the upstream regulatory region can be altered to thereby alter the expression of the endogenous HEAT protein). In the homologous recombination nucleic acid molecule, the altered portion of the HEAT gene is flanked at its 5′ and 3′ ends by additional nucleic acid sequence of the HEAT gene to allow for homologous recombination to occur between the exogenous HEAT gene carried by the homologous recombination nucleic acid molecule and an endogenous HEAT gene in a cell, e.g., an embryonic stem cell. The additional flanking HEAT nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced HEAT gene has homologously recombined with the endogenous HEAT gene are selected (see e.g., Li, E. et al. (1992) Cell 69:915). The selected cells can then be injected into a blastocyst of an animal (e.g., a mouse) to form aggregation chimeras (see e.g., Bradley, A., in Teratocarcinomas and Embryonic Stem Cells: A Practical Approach, Robertson, E. J. ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Curr. Opin. Biotechnol 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G_{O} phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The HEAT nucleic acid molecules, proteins, fragments thereof, anti-HEAT antibodies, and HEAT modulators (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a HEAT protein or an anti-HEAT antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

In certain embodiments of the invention, a modulator of HEAT activity is administered in combination with other agents (e.g., a small molecule), or in conjunction with another, complementary treatment regime. For example, in one embodiment, a modulator of HEAT activity is used to treat a cardiovascular disorder (e.g., atherosclerosis, hypertension, and/or vascular disease). Accordingly, modulation of HEAT activity may be used in conjunction with, for example, another agent used to treat the disorder. For example, non-limiting examples of agents used to treat cardiovascular disorders include niacin; clofibrate; bile acid binding resins (e.g., cholestipol and cholestyramine); neomycin; statins (e.g., atorvastatin, fluvastatin, lovastatin, pravastatin, and simvastatin); diuretics (e.g., loop diuretics such as furosemide, bumetanide, torsemide, and ethacryinic acid; thiazide diuretics such as hydrochlorothiazide, chlorothiazide, methyclothiazide, and bendroflumethiazide; osmotic diuretics such as mannitol, glycerine, and isosorbide; potassium-sparing diuretics such as spironolactone; and sodium channel blockers such as amiloride and triamterene); angiotensin converting enzyme inhibitors (e.g., enalapril, capoten, and lisinopril); angiotensin II receptor blockers (e.g., losartan, valsartan, and candasartan); aldosterone antagonists (e.g., spironolactone); alpha 2 adrenergic agonists (e.g., methyldopa and clonidine); alpha 1 adrenergic blockers (e.g., prazosin and terazosin); beta blockers (e.g., propranolol, metoprolol, and atenolol); combination alpha/beta blockers (e.g., carvedilol); nitrates (e.g., nitroglycerin and isosorbide dinitrate); calcium channel blockers (e.g., verapamil, nicardipine, amlopidine, diltiazem, and nifedipine); digoxin; folic acid; sodium channel blockers (e.g., quinidine, lidocaine, and procainamide); vasodilators (e.g., minoxidil and hydralizine); thrombolytic agents (e.g., streptokinase and urokinase); tissue plasminogen activators (e.g., alteplase, reteplase, and tenecteplase); antiplatelet agents (e.g., aspirin, clopidogrel, and dipyridimole); and anticoagulants (e.g., heparin, enoxaparin, dalteparin, ardeparin, and warfarin).

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, dauniorubicin, dihydroxy anthracin dione, mitoxantrone, mithraniycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodianiine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al. “Monoclonal Antibodies For Immunotargeting Of Drugs In Cancer Therapy” in Monoclonal Antibodies And Cancer Therapy, Reisfeld et al. (eds.), pp. 243-56 (Alan R. Liss, Inc. 1985); Hellstrom et al. “Antibodies For Drug Delivery” in Controlled Drug Delivery (2nd Ed.), Robinson et al. (eds.), pp. 623-53 (Marcel Dekker, Inc. 1987); Thorpe “Antibody Carriers Of Cytotoxic Agents In Cancer Therapy: A Review” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy” in Monoclonal Antibodies For Cancer Detection And Therapy, Baldwin et al. (eds.), pp. 303-16 (Academic Press 1985); and Thorpe et al. “The Preparation And Cytotoxic Properties Of Antibody-Toxin Conjugates” Immunol Rev. 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, protein fragments, antibodies, peptides, peptidomimetics, and small molecules described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a HEAT protein of the invention has one or more of the following activities: (i) interaction with a HEAT substrate or target molecule (e.g., a Ca²⁺ ion; ATP; or a non-HEAT protein;); (ii) transport of a HEAT substrate or target molecule (e.g., a Ca²⁺ ion) from one side of a biological membrane to the other; (iii) ability to be phosphorylated or dephosphorylated; (iv) adoption of an E1 conformation or an E2 conformation; (v) conversion of a HEAT substrate or target molecule to a product (e.g., hydrolysis of ATP to ADP and free phosphate); (vi) interaction with a second non-HEAT protein; (vii) modulation of intra- or inter-cellular signaling and/or gene transcription (e.g., either directly or indirectly); (viii) modulation of vascular smooth muscle tone; (ix) modulation of cellular growth and/or proliferation; and/or (x) modulation of angiogenesis.

The isolated nucleic acid molecules of the invention can be used, for example, to express HEAT protein (e.g., via a recombinant expression vector in a host cell in gene therapy applications), to detect HEAT mRNA (e.g., in a biological sample) or a genetic alteration in a HEAT gene, and to modulate HEAT activity, as described further below. The HEAT proteins can be used to treat disorders characterized by insufficient or excessive HEAT protein activity or HEAT nucleic acid expression, for example, cardiovascular disorders.

In addition, the HEAT proteins can be used to screen for naturally occurring HEAT substrates, to screen for drugs or compounds which modulate HEAT activity, as well as to treat disorders characterized by insufficient or excessive production of HIEAT protein or production of HEAT protein forms which have decreased, aberrant or unwanted activity compared to HEAT wild type protein (e.g., a cardiovascular disorder).

Moreover, the anti-HEAT antibodies of the invention can be used to detect and isolate HEAT proteins, regulate the bioavailability of HEAT proteins, and modulate HEAT activity.

A. Screening Assays

The invention provides a method (also referred to herein as a “screening assay”) for identifying modulators, i.e., candidate or test compounds or agents (e.g., peptides, peptidomimetics, small molecules or other drugs) which bind to HEAT proteins, have a stimulatory or inhibitory effect on, for example, HEAT expression or HEAT activity, or have a stimulatory or inhibitory effect on, for example, the expression or activity of a HEAT substrate.

In one embodiment, the invention provides assays for screening candidate or test compounds which are substrates of a HEAT protein or polypeptide or biologically active portion thereof. In another embodiment, the invention provides assays for screening candidate or test compounds which bind to or modulate the activity of a HEAT protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:45).

Examples of methods for the synthesis of molecular libraries can be found in the art, for example, in: DeWitt et al. (1993) Proc. Natl. Acad. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten ( 992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.).

In one embodiment, an assay is a cell-based assay in which a cell which expresses a HEAT protein or biologically active portion thereof is contacted with a test compound and the ability of the test compound to modulate HEAT activity is determined. Determining the ability of the test compound to modulate HEAT activity can be accomplished by monitoring, for example: (i) interaction with a HEAT substrate or target molecule (e.g., a Ca²⁺ ion; ATP; or a non-HEAT protein;); (ii) transport of a HEAT substrate or target molecule (e.g., a Ca²⁺ ion) from one side of a biological membrane to the other; (iii) ability to be phosphorylated or dephosphorylated; (iv) adoption of an E1 conformation or an E2 conformation; (v) conversion of a HEAT substrate or target molecule to a product (e.g., hydrolysis of ATP to ADP and free phosphate); (vi) interaction with a second non-HEAT protein; (vii) modulation of intra- or inter-cellular signaling and/or gene transcription (e.g., either directly or indirectly); (viii) modulation of vascular smooth muscle tone; (ix) modulation of cellular growth and/or proliferation; and/or (x) modulation of angiogenesis.

Determining the ability of the test compound to modulate angiogenesis can be accomplished by testing the compound in a chicken choirioallantoic membrane (CAM) assay. In a CAM assay, the ability of test compounds to modulate bFGF induced angiogenesis from the CAM, can be determined. The CAM assay is performed essentially as described in Liekens, S. et al. (1997) Oncology Res. 9:173-181, the contents of which are incorporated herein by reference, and may be performed with the modifications described below. Briefly, fresh fertilized chicken eggs are incubated for 3 days at 37° C. On the third day, the shell is cracked and the egg is placed into a tissue culture plate and incubated at 38° C. For the assay, bFGF and the compound to be tested are attached on a matrix of collagen on a nylon mesh. The mesh is then used to cover the chorioallantoic membrane and the eggs are incubated at 37° C. If angiogenesis occurs, new capillaries form and grow through the mesh within 24 hours. The ability of the test compounds (at various concentrations) to modulate the bFGF-induced angiogenesis can then be determined.

The ability of a HEAT protein to be phosphorylated (e.g. be autophosphorylated) can be determined by, for example, an in vitro kinase assay. Briefly, a HEAT protein, e.g., an immunoprecipitated HEAT protein from a cell line expressing such a protein, can be incubated with radioactive ATP, e.g., [γ-³²P]ATP, in a buffer containing MgCl₂ and MnCl₂, e.g., 10 mM MgCl₂ and 5 mM MnCl₂. Following the incubation, the immunoprecipitated HEAT protein can be separated by SDS-polyacrylamide gel electrophoresis under reducing conditions, transferred to a membrane, e.g., a PVDF membrane, and autoradiographed. The appearance of detectable bands on the autoradiograph indicates that the HEAT protein has been phosphorylated. Phosphoaminoacid analysis of the phosphorylated HEAT protein can also be performed in order to determine which residues on the HEAT protein are phosphorylated (e.g., to test whether the D amino acid residue in the E1-E2 ATPases phosphorylation site has been phosphorylated). Briefly, the radiophosphorylated protein band can be excised from the SDS gel and subjected to partial acid hydrolysis. The products can then be separated by one-dimensional electrophoresis and analyzed on, for example, a phosphorimager and compared to ninhydrin-stained phosphoaminoacid standards.

The ability of the test compound to modulate HEAT binding to a substrate or to bind to another HEAT protein or subunit can also be determined. Determining the ability of the test compound to modulate HEAT binding to a substrate can be accomplished, for example, by coupling the HEAT substrate with a radioisotope or enzymatic label such that binding of the HEAT substrate to HEAT can be determined by detecting the labeled HEAT substrate in a complex. Alternatively, HEAT could be coupled with a radioisotope or enzymatic label to monitor the ability of a test compound to modulate HEAT binding to a HEAT substrate in a complex. Determining the ability of the test compound to bind HEAT can be accomplished, for example, by coupling the compound with a radioisotope or enzymatic label such that binding of the compound to HEAT can be determined by detecting the labeled HEAT compound in a complex. For example, compounds (e.g., HEAT substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g, a HEAT substrate) to interact with HEAT without the labeling of any of the interactants. For example, a microphysiometer can be used to detect the interaction of a compound with HEAT without the labeling of either the compound or the HEAT. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and HEAT.

In another embodiment, an assay is a cell-based assay comprising contacting a cell expressing a HEAT target molecule (e.g., a HEAT substrate) with a test compound and determining the ability of the test compound to modulate (e.g., stimulate or inhibit) the activity of the HEAT target molecule. Determining the ability of the test compound to modulate the activity of a HEAT target molecule can be accomplished, for example, by determining the ability of a HEAT protein to bind to or interact with the HEAT target molecule, by determining the cellular location of the target molecule, or determining whether the target molecule (e.g., ATP) has been hydrolyzed.

In one embodiment, the ability of a test compound to modulate the activity of a HEAT protein or a HEAT target molecule can be determined by measuring microvessel contraction (as described in, for example, Example 4 and in Bischoff, A. et al. (2000) Br. J. Pharmacol. 130:1871-1877 and in Volpe and Cosentino (2000) J. Cardiovasc. Pharmacol. 35 (4 Suppl 2):S45-48). In another embodiment, the ability of a test compound to modulate the activity of a HEAT protein or a HEAT target molecule can be determined by measuring intracellular Ca²⁺ concentration (as described in, for example, Example 5 and in Bischoff, A. et al. (2000) supra). In still another embodiment, the ability of a test compound to modulate the activity of a HEAT protein or a HEAT target molecule can be determined by measuring calcium transport by the HEAT protein (as described in, for example, Example 6 and in Maruyama, K. and MacLennan, D. H. (1988) Proc. Natl. Acad. Sci. USA 85:3314-3318).

Determining the ability of the HEAT protein, or a biologically active fragment thereof, to bind to or interact with a HEAT target molecule can be accomplished by one of the methods described above for determining direct binding. In a preferred embodiment, determining the ability of the HEAT protein to bind to or interact with a HEAT target molecule can be accomplished by determining the activity of the target molecule. For example, the activity of the target molecule can be determined by detecting the cellular location of the target molecule, detecting a metabolite of the target molecule (e.g., detecting the byproducts of ATP hydrolysis), detecting catalytic/enzymatic activity of the target molecule upon an appropriate substrate, detecting the induction of a reporter gene (comprising a target-responsive regulatory element operatively linked to a nucleic acid encoding a detectable marker, e.g., luciferase), or detecting a target-regulated cellular response (e.g., a change in vascular smooth muscle tone).

In yet another embodiment, an assay of the present invention is a cell-free assay in which a HEAT protein or biologically active portion thereof is contacted with a test compound and the ability of the test compound to bind to the HEAT protein or biologically active portion thereof is determined. Preferred biologically active portions of the HEAT proteins to be used in assays of the present invention include fragments which participate in interactions with non-HEAT molecules, e.g., fragments with high surface probability scores (see, for example, FIGS. 2, 13, and 24). Binding of the test compound to the HEAT protein can be determined either directly or indirectly as described above. In a preferred embodiment, the assay includes contacting the HEAT protein or biologically active portion thereof with a known compound which binds HEAT to form an assay mixture, contacting the assay mixture with a test compound, and determining the ability of the test compound to interact with a HEAT protein, wherein determining the ability of the test compound to interact with a HEAT protein comprises determining the ability of the test compound to preferentially bind to HEAT or biologically active portion thereof as compared to the known compound.

In another embodiment, the assay is a cell-free assay in which a HEAT protein or biologically active portion thereof is contacted with a test compound and the ability of the test compound to modulate (e.g., stimulate or inhibit) the activity of the HEAT protein or biologically active portion thereof is determined. Determining the ability of the test compound to modulate the activity of a HEAT protein can be accomplished, for example, by determining the ability of the HEAT protein to bind to a HEAT target molecule by one of the methods described above for determining direct binding. Determining the ability of the HEAT protein to bind to a HEAT target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a HEAT protein can be accomplished by determining the ability of the HEAT protein to further modulate the activity of a downstream effector of a HEAT target molecule. For example, the activity of the effector molecule on an appropriate target can be determined or the binding of the effector to an appropriate target can be determined as previously described.

In yet another embodiment, the cell-free assay involves contacting a HEAT protein or biologically active portion thereof with a known compound which binds the HEAT protein to form an assay mixture, contacting the assay mixture with a test compound, and determining the ability of the test compound to interact with the HEAT protein, wherein determining the ability of the test compound to interact with the HEAT protein comprises determining the ability of the HEAT protein to preferentially bind to or modulate the activity of a HEAT target molecule.

The cell-free assays of the present invention are amenable to use of both soluble and/or membrane-bound forms of isolated proteins (e.g., HEAT proteins or biologically active portions thereof). In the case of cell-free assays in which a membrane-bound form of an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either HEAT or its target molecule to facilitate separation of complexed from uncomplexed forms of one or both of the proteins, as well as to accommodate automation of the assay. Binding of a test compound to a HEAT protein, or interaction of a HEAT protein with a substrate or target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/HEAT fusion proteins or glutathione-S-transferase/target fusion proteins can be adsorbed onto glutathione sepharose beads (Sigma Chemical, St. Louis, Mo.) or glutathione derivatized micrometer plates, which are then combined with the test compound or the test compound and either the non-adsorbed target protein or HEAT protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of HEAT binding or activity determined using standard techniques.

Other techniques for immobilizing proteins on matrices can also be used in the screening assays of the invention. For example, either a HEAT protein or a HEAT substrate or target molecule can be immobilized utilizing conjugation of biotin and streptavidin. Biotinylated HEAT protein, substrates, or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with HEAT protein or target molecules but which do not interfere with binding of the HEAT protein to its target molecule can be derivatized to the wells of the plate, and unbound target or HEAT protein trapped in the wells by antibody conjugation. Methods for detecting such complexes, in addition to those described above for the GST-immobilized complexes, include immunodetection of complexes using antibodies reactive with the HEAT protein or target molecule, as well as enzyme-linked assays which rely on detecting an enzymatic activity associated with the HEAT protein or target molecule.

In another embodiment, modulators of HEAT expression are identified in a method wherein a cell is contacted with a candidate compound and the expression of HEAT mRNA or protein in the cell is determined. The level of expression of HEAT mRNA or protein in the presence of the candidate compound is compared to the level of expression of HEAT mRNA or protein in the absence of the candidate compound. The candidate compound can then be identified as a modulator of HEAT expression based on this comparison. For example, when expression of HEAT mRNA or protein is greater (statistically significantly greater) in the presence of the candidate compound than in its absence, the candidate compound is identified as a stimulator of HEAT mRNA or protein expression. Alternatively, when expression of HEAT mRNA or protein is less (statistically significantly less) in the presence of the candidate compound than in its absence, the candidate compound is identified as an inhibitor of HEAT mRNA or protein expression. The level of HEAT mRNA or protein expression in the cells can be determined by methods described herein for detecting HEAT mRNA or protein.

In yet another aspect of the invention, the HEAT proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300) to identify other proteins which bind to or interact with HEAT (“HEAT-binding proteins” or “HEAT-bp”) and are involved in HEAT activity. Such HEAT-binding proteins are also likely to be involved in the propagation of signals by the HEAT proteins or HEAT targets as, for example, downstream elements of a HEAT-mediated signaling pathway. Alternatively, such HEAT-binding proteins may be HEAT inhibitors.

The two-hybrid system is based on the modular nature of most transcription factors, which consist of separable DNA-binding and activation domains. Briefly, the assay utilizes two different DNA constructs. In one construct, the gene that codes for a HEAT protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a HEAT-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the HEAT protein.

In another aspect, the invention pertains to a combination of two or more of the assays described herein. For example, a modulating agent can be identified using a cell-based or a cell-free assay, and the ability of the agent to modulate the activity of a HEAT protein can be confirmed in vivo, e.g., in an animal such as an animal model for vascular disease, hypertension, angiogenesis, and/or cellular transformation or tumorigenesis.

For example, the following animal models may be used in the methods of the invention: the mouse model of cardiomyopathy in the HIV-1 transgenic mouse treated with zidovudine (Lewis, W. et al. (2000) Lab Invest. 80:187-97); the pressure-overloaded guinea pig model with cardiac hypertrophy and failure (Ahmmed, G. U. et al. (2000) Circ. Res. 86:558-70); the hypertensive transgenic mouse model that lacks fat and has lipoatrophic diabetes (Reitmann, M. L. et al. (1999) Ann N.Y. Acad. Sci. 192:289-96; Moitra J. et al. (1998) Genes Dev. 12:3168-81); various mouse models of hypertension (Cvetokovic, B. and Sigmund, C. D. (2000) Kidney Int. 57:863-74; Merrill, D. C. et al. (1997) Proc. Assoc. Am. Physicians 109:533-46); various rat models for hypertension (Rapp, J. P. (2000) Physiol. Rev. 80:135-72; Yamori, Y. (1999) Clin. Exp. Pharmacol. Physiol. 26:568-72; Nara, Y. et al. (1999) Clin. Exp. Pharmacol. Physiol. 17:481-7; Boulanger, C. M. (1999) J. Mol. Cell. Cardiol. 31:39-49; Wookey, P. J. et al. (1998) Miner Electrolyte Metab. 24:389-99; Aitman, T. J. (1998) Pathol. Biol. (Paris) 46:693-4; Engler, S. et al. (1998) Regul. Pept, 77:3-8; Zolk, O. et al. (1998) Cardiovasc. Res. 39:242-56; Pinto, Y. M. et al. (1998) Cardiovasc. Res. 39:77-88; Yagil, Y. and Yagil, C. (1998) Kidney Int. 53:1493-500; Packer, C. S. (1994) Proc. Soc. Exp. Biol. Med. 207:148-74; Griffith, S. L. et al. (1994) J. Appl. Physiol. 77:406-14); animal models of lower extremity chronic venous disease (Dalsing, M. C. et al. (1998) Ann. Vasc. Surg. 12:487-94); the pecten oculi of the chicken, a model system for studying vascular differentiation and barrier maturation (Wolburg, H. et al. (1999) Int. Rev. Cytol. 187:111-59); a VEGF transgenic animal model for atherosclerosis and angiogenesis (Sueishi, K. et al. (1997) Ann N.Y. Acad. Sci. 811:311-324); the chick embryo chorioallantoic membrane, a model for in vivo research on angiogenesis (Ribatti, D. et al. (1996) Int. J. Dev. Biol. 40:1189-97); various rat models of angiogenesis (Fan, T. P. et al. (1992) E. X. S. 61:308-14; Norrby, K. (1992) E. X. S. 61:282-6); the porcine model for coronary artery spasm (Shimokawa, H. (2000) Jpn. Circ. J. 64:1-12; Kuga, T. et al. (2000) J. Cardiovasc. Pharmacol. 35:822-8; Kandabashi, T. et al. (2000) Circulation 101:1319-23); various animal models of neointima formation in atherosclerosis-prone arteries (De Meyer, G. R. and Bult, H. (1997) Vasc. Med. 2:179-89); the hamster cheek pouch model for vascular smooth muscle function and microcirculation research (Svensjo, E. (1990) Eur. Respir. J. Suppl. 12:595s-600s); and the squid axon model for studying plasma membrane mechanism for calcium regulation (DiPolo, R. and Beauge, L. (1987) Hypertension 10:115-9). There are also numerous animal models for tumorigenesis, including: mice that develop spontaneous tumors, either naturally, or as a result of addition of an exogenous, tumor causing transgene, a knock-out of an endogenous gene, or injection of exogenous tumor cells, and mice that develop tumors as a result of infection with oncogene-containing viruses.

This invention further pertains to novel agents identified by the above-described screening assays. Accordingly, it is within the scope of this invention to further use an agent identified as described herein in an appropriate animal model (e.g., an animal model such as any of those described above). For example, an agent identified as described herein (e.g., a HEAT modulating agent, an antisense HEAT nucleic acid molecule, a HEAT-specific antibody, or a HEAT binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

In another aspect, cell-based systems, as described herein, may be used to identify compounds which may act to ameliorate cardiovascular disease symptoms. For example, such cell systems may be exposed to a compound, suspected of exhibiting an ability to ameliorate cardiovascular disease symptoms, at a sufficient concentration and for a time sufficient to elicit such an amelioration of cardiovascular disease symptoms in the exposed cells. After exposure, the cells are examined to determine whether one or more of the cardiovascular disease cellular phenotypes has been altered to resemble a more normal or more wild type, non-cardiovascular disease phenotype. Cellular phenotypes that are associated with cardiovascular disease states include aberrant vascular tone, angiogenesis, or tube formation.

In addition, animal-based cardiovascular disease systems, such as those described herein, may be used to identify compounds capable of ameliorating cardiovascular disease symptoms. Such animal models may be used as test substrates for the identification of drugs, pharmaceuticals, therapies, and interventions which may be effective in treating cardiovascular disease. For example, animal models may be exposed to a compound, suspected of exhibiting an ability to ameliorate cardiovascular disease symptoms, at a sufficient concentration and for a time sufficient to elicit such an amelioration of cardiovascular disease symptoms in the exposed animals. The response of the animals to the exposure may be monitored by assessing the reversal of disorders associated with cardiovascular disease, for example, by measuring blood pressure, by measuring the degree of vascularization of a tumor or eye, or by measuring the size of atherosclerotic plaques before and after treatment. In addition, the animals may be monitored by assessing the reversal of disorders associated with cardiovascular disease, for example, reduction in hypertension, reduction in atherosclerosis, or reduction in tumor burden, tumor size, and invasive and/or metastatic potential before and after treatment.

With regard to intervention, any treatments which reverse any aspect of cardiovascular disease symptoms should be considered as candidates for human cardiovascular disease therapeutic intervention. Dosages of test agents may be determined by deriving dose-response curves.

Additionally, gene expression patterns may be utilized to assess the ability of a compound to ameliorate cardiovascular disease symptoms. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Such conditions may include, but are not limited to, the presence of hypertension, the presence of atherosclerotic plaques, or the presence of a vascularized tumor, e.g, a colon or lung tumor, including any of the control or experimental conditions described herein. Other conditions may include, for example, tube formation or shear stress. Gene expression profiles may be generated, for example, by utilizing a differential display procedure, Northern analysis and/or RT-PCR. In one embodiment, HEAT gene sequences may be used as probes and/or PCR primers for the generation and corroboration of such gene expression profiles.

Gene expression profiles may be characterized for known states, either cardiovascular disease or normal, within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of a cardiovascular disease model system to more closely resemble the control system. Administration of a compound may, alternatively, cause the gene expression profile of a control system to begin to mimic a cardiovascular disease state. Such a compound may, for example, be used in further characterizing the compound of interest, or may be used in the generation of additional animal models.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the HEAT nucleotide sequences, described herein, can be used to map the location of the HEAT genes on a chromosome. The mapping of the HEAT sequences to chromosomes is an important first step in correlating these sequences with genes associated with disease.

Briefly, HEAT genes can be mapped to chromosomes by preparing PCR primers (preferably 15-25 bp in length) from the HEAT nucleotide sequences. Computer analysis of the HEAT sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the HEAT sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes (D'Eustachio, P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the HEAT nucleotide sequences to design oligonucleotide primers, sublocalization can be achieved with panels of fragments from specific chromosomes. Other mapping strategies which can similarly be used to map a HEAT sequence to its chromosome include in situ hybridization (described in Fan, Y. et al. (1990) Proc. Natl. Acad. Sci. USA 87:6223-27), pre-screening with labeled flow-sorted chromosomes, and pre-selection by hybridization to chromosome-specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data (such data are found, for example, in McKusick, V., Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature 325:783-787.

Moreover, differences in the DNA sequences between individuals affected and unaffected with a disease associated with the HEAT gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The HEAT sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the HEAT nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The HEAT nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, 5, or 8 can comfortably provide positive individual identification with a panel of perhaps 10 to 1,000 primers which each yield a noncoding amplified sequence of 100 bases. If predicted coding sequences, such as those in SEQ ID NO:3, 7, or 10 are used, a more appropriate number of primers for positive individual identification would be 500-2,000.

If a panel of reagents from HEAT nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of Partial HEAT Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1, 5, or 8 are particularly appropriate for this use as greater numbers of polymorphisms occur in the noncoding regions, making it easier to differentiate individuals using this technique. Examples of polynucleotide reagents include the HEAT nucleotide sequences or portions thereof, e.g., fragments derived from the noncoding regions of SEQ ID NO:1, 5, or 8 having a length of at least 20 bases, preferably at least 30 bases.

The HEAT nucleotide sequences described herein can further be used to provide polynucleotide reagents, e.g., labeled or labelable probes which can be used in, for example, an in situ hybridization technique, to identify a specific tissue, e.g., vascular smooth muscle tissue. This can be very useful in cases where a forensic pathologist is presented with a tissue of unknown origin. Panels of such HEAT probes can be used to identify tissue by species and/or by organ type.

In a similar fashion, these reagents, e.g., HEAT primers or probes can be used to screen tissue culture for contamination (i.e., screen for the presence of a mixture of different types of cells in a culture).

C. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining HEAT protein and/or nucleic acid expression as well as HEAT activity, in the context of a biological sample (e.g., blood, serum, cells, or tissue) to thereby determine whether an individual is afflicted with a disease or disorder, or is at risk of developing a disorder, associated with aberrant or unwanted HEAT expression or activity. The invention also provides for prognostic (or predictive) assays for determining whether an individual is at risk of developing a disorder associated with HEAT protein, nucleic acid expression, or activity. For example, mutations in a HEAT gene can be assayed in a biological sample. Such assays can be used for prognostic or predictive purpose to thereby prophylactically treat an individual prior to the onset of a disorder characterized by or associated with HEAT protein, nucleic acid expression or activity.

Another aspect of the invention pertains to monitoring the influence of agents (e.g., drugs, compounds) on the expression or activity of HEAT in clinical trials.

These and other agents are described in further detail in the following sections.

1. Diagnostic Assays

An exemplary method for detecting the presence or absence of HEAT protein, polypeptide or nucleic acid in a biological sample involves obtaining a biological sample from a test subject and contacting the biological sample with a compound or an agent capable of detecting HEAT protein, polypeptide or nucleic acid (e.g., mRNA, genomic DNA) that encodes HEAT protein such that the presence of HEAT protein or nucleic acid is detected in the biological sample. In another aspect, the present invention provides a method for detecting the presence of HEAT activity in a biological sample by contacting the biological sample with an agent capable of detecting an indicator of HEAT activity such that the presence of HEAT activity is detected in the biological sample. A preferred agent for detecting HEAT mRNA or genomic DNA is a labeled nucleic acid probe capable of hybridizing to HEAT mRNA or genomic DNA. The nucleic acid probe can be, for example, a full-length HEAT nucleic acid, such as the nucleic acid of SEQ ID NO:1, 3, 5, 7, 8, or 10, or the DNA insert of the plasmid deposited with ATCC as Accession Number ______, ______, or ______, or a portion thereof, such as an oligonucleotide of at least 15, 30, 50, 100, 250 or 500 nucleotides in length and sufficient to specifically hybridize under stringent conditions to HEAT mRNA or genomic DNA. Other suitable probes for use in the diagnostic assays of the invention are described herein.

A preferred agent for detecting HEAT protein is an antibody capable of binding to HEAT protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect HEAT mRNA, protein, or genomic DNA in a biological sample in vitro as well as in vivo. For example, in vitro techniques for detection of HEAT mRNA include Northern hybridizations and in situ hybridizations. In vitro techniques for detection of HEAT protein include enzyme linked immunosorbent assays (ELISAs), Western blots, immunoprecipitations and immunofluorescence. In vitro techniques for detection of HEAT genomic DNA include Southern hybridizations. Furthermore, in vivo techniques for detection of a HEAT protein include introducing into a subject a labeled anti-HEAT antibody. For example, the antibody can be labeled with a radioactive marker whose presence and location in a subject can be detected by standard imaging techniques.

The present invention also provides diagnostic assays for identifying the presence or absence of a genetic alteration characterized by at least one of (i) aberrant modification or mutation of a gene encoding a HEAT protein; (ii) aberrant expression of a gene encoding a HEAT protein; (iii) mis-regulation of the gene; and (iii) aberrant post-translational modification of a HEAT protein, wherein a wild-type form of the gene encodes a protein with a HEAT activity. “Misexpression or aberrant expression”, as used herein, refers to a non-wild type pattern of gene expression, at the RNA or protein level. It includes, but is not limited to, expression at non-wild type levels (e.g., over or under expression); a pattern of expression that differs from wild type in terms of the time or stage at which the gene is expressed (e.g., increased or decreased expression (as compared with wild type) at a predetermined developmental period or stage); a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene (e.g., a pattern of increased or decreased expression (as compared with wild type) in the presence of an increase or decrease in the strength of the stimulus).

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting HEAT protein, mRNA, or genomic DNA, such that the presence of HEAT protein, mRNA or genomic DNA is detected in the biological sample, and comparing the presence of HEAT protein, mRNA or genomic DNA in the control sample with the presence of HEAT protein, mRNA or genomic DNA in the test sample.

The invention also encompasses kits for detecting she presence of HEAT in a biological sample. For example, the kit can comprise a labeled compound or agent capable of detecting HEAT protein or mRNA in a biological sample; means for determining the amount of HEAT in the sample; and means for comparing the amount of HEAT in the sample with a standard. The compound or agent can be packaged in a suitable container. The kit can further comprise instructions for using the kit to detect HEAT protein or nucleic acid.

2. Prognostic Assays

The diagnostic methods described herein can furthermore be utilized to identify subjects having or at risk of developing a disease or disorder associated with aberrant or unwanted HEAT expression or activity. As used herein, the term “aberrant” includes a HEAT expression or activity which deviates from the wild type HEAT expression or activity. Aberrant expression or activity includes increased or decreased expression or activity, as well as expression or activity which does not follow the wild type developmental pattern of expression or the subcellular pattern of expression. For example, aberrant HEAT expression or activity is intended to include the cases in which a mutation in the HEAT gene causes the HEAT gene to be under-expressed or over-expressed and situations in which such mutations result in a non-functional HEAT protein or a protein which does not function in a wild-type fashion, e.g., a protein which does not interact with or transport a HEAT substrate, or one which interacts with or transports a non-HEAT substrate. As used herein, the term “unwanted” includes an unwanted phenomenon involved in a biological response such as deregulated cation transport. For example, the term unwanted includes a HEAT expression or activity which is undesirable in a subject.

The assays described herein, such as the preceding diagnostic assays or the following assays, can be utilized to identify a subject having or at risk of developing a disorder associated with a misregulation in HEAT protein activity or nucleic acid expression, such as a vascular disorder, an angiogenesis disorder, or a cell growth or proliferation disorder. Alternatively, the prognostic assays can be utilized to identify a subject having or at risk for developing a disorder associated with a misregulation in HEAT protein activity or nucleic acid expression, such as a vascular disorder, an angiogenesis disorder, or a cell growth or proliferation disorder. Thus, the present invention provides a method for identifying a disease or disorder associated with aberrant or unwanted HEAT expression or activity in which a test sample is obtained from a subject and HEAT protein or nucleic acid (e.g., mRNA or genomic DNA) is detected, wherein the presence of HEAT protein or nucleic acid is diagnostic for a subject having or at risk of developing a disease or disorder associated with aberrant or unwanted HEAT expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted HEAT expression or activity. For example, such methods can be used to determine whether a subject can be effectively treated with an agent for a drug or toxin sensitivity disorder or a cell proliferation and/or differentiation disorder. Thus, the present invention provides methods for determining whether a subject can be effectively treated with an agent for a disorder associated with aberrant or unwanted HEAT expression or activity in which a test sample is obtained and HEAT protein or nucleic acid expression or activity is detected (e.g., wherein the abundance of HEAT protein or nucleic acid expression or activity is diagnostic for a subject that can be administered the agent to treat a disorder associated with aberrant or unwanted HEAT expression or activity).

The methods of the invention can also be used to detect genetic alterations in a HEAT gene, thereby determining if a subject with the altered gene is at risk for a disorder characterized by misregulation in HEAT protein activity or nucleic acid expression, such as a vascular disorder, an angiogenesis disorder, or a cell growth or proliferation disorder. In preferred embodiments, the methods include detecting, in a sample of cells from the subject, the presence or absence of a genetic alteration characterized by at least one of an alteration affecting the integrity of a gene encoding a HEAT-protein, or the mis-expression of the HEAT gene. For example, such genetic alterations can be detected by ascertaining the existence of at least one of 1) a deletion of one or more nucleotides from a HEAT gene; 2) an addition of one or more nucleotides to a HEAT gene; 3) a substitution of one or more nucleotides of a HEAT gene, 4) a chromosomal rearrangement of a HEAT gene; 5) an alteration in the level of a messenger RNA transcript of a HEAT gene, 6) aberrant modification of a HEAT gene, such as of the methylation pattern of the genomic DNA, 7) the presence of a non-wild type splicing pattern of a messenger RNA transcript of a HEAT gene, 8) a non-wild type level of a HEAT-protein, 9) allelic loss of a HEAT gene, and 10) inappropriate post-translational modification of a HEAT-protein. As described herein, there are a large number of assays known in the art which can be used for detecting alterations in a HEAT gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the HEAT-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a HEAT gene under conditions such that hybridization and amplification of the HEAT gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a HEAT gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in HEAT can be identified by hybridizing a sample and control nucleic acids, e.g., DNA or RNA, to high density arrays containing hundreds or thousands of oligonucleotides probes (Cronin, M. T. et al. (1 996) Hum. Mutat. 7:244-255; Kozal, M. J. et al. (1 996) Nat. Med. 2:753-759). For example, genetic mutations in HEAT can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the HEAT gene and detect mutations by comparing the sequence of the sample HEAT with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays (Naeve, C. W. (1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the HEAT gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type HEAT sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in HEAT cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a HEAT sequence, e.g., a wild-type HEAT sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in HEAT genes. For example, single strand conformation polymorphism (SSCP) may be used to detect differences in electrophoretic mobility between mutant and wild type nucleic acids (Orita et al. (1989) Proc. Natl. Acad. Sci. USA 86:2766, see also Cotton (1993) Mutat. Res. 285:125-144; and Hayashi (1992) Genet. Anal. Tech. Appl. 9:73-79). Single-stranded DNA fragments of sample and control HEAT nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a HEAT gene.

Furthermore, any cell type or tissue in which HEAT is expressed (e.g., vessels, endothelial cells, and/or vascular smooth muscle cells) may be utilized in the prognostic assays described herein.

3. Monitoring of Effects During Clinical Trials

Monitoring the influence of agents (e.g., drugs) on the expression or activity of a HEAT protein (e.g., the modulation of cellular signaling, Ca²⁺ transport, gene expression, vascular smooth muscle tone regulation, angiogenesis, and/or cell growth or proliferation mechanisms) can be applied not only in basic drug screening, but also in clinical trials. For example, the effectiveness of an agent determined by a screening assay as described herein to increase HEAT gene expression, protein levels, or upregulate HEAT activity, can be monitored in clinical trials of subjects exhibiting decreased HEAT gene expression, protein levels, or downregulated HEAT activity. Alternatively, the effectiveness of an agent determined by a screening assay to decrease HEAT gene expression, protein levels, or downregulate HEAT activity, can be monitored in clinical trials of subjects exhibiting increased HEAT gene expression, protein levels, or upregulated HEAT activity. In such clinical trials, the expression or activity of a HEAT gene, and preferably, other genes that have been implicated in, for example, a cardiovascular disorder can be used as a “read out” or markers of the phenotype of a particular cell.

For example, and not by way of limitation, genes, including HEAT, that are modulated in cells by treatment with an agent (e.g., compound, drug or small molecule) which modulates HEAT activity (e.g., identified in a screening assay as described herein) can be identified. Thus, to study the effect of agents on cardiovascular disorders (e.g., disorders characterized by deregulated cellular signaling, Ca²⁺ transport, gene expression, vascular smooth muscle tone regulation, angiogenesis, and/or cell growth or proliferation mechanisms), for example, in a clinical trial, cells can be isolated and RNA prepared and analyzed for the levels of expression of HEAT and other genes implicated in the cardiovascular disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of HEAT or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a HEAT protein, mRNA, or genomic DNA in the preadministration sample; (iii) obtaining one or more post-administration samples from the subject; (iv) detecting the level of expression or activity of the HEAT protein, mRNA, or genomic DNA in the post-administration samples; (v) comparing the level of expression or activity of the HEAT protein, mRNA, or genomic DNA in the pre-administration sample with the HEAT protein, mRNA, or genomic DNA in the post administration sample or samples; and (vi) altering the administration of the agent to the subject accordingly. For example, increased administration of the agent may be desirable to increase the expression or activity of HEAT to higher levels than detected, i.e., to increase the effectiveness of the agent. Alternatively, decreased administration of the agent may be desirable to decrease expression or activity of HEAT to lower levels than detected, i.e., to decrease the effectiveness of the agent. According to such an embodiment, HEAT expression or activity may be used as an indicator of the effectiveness of an agent, even in the absence of an observable phenotypic response.

D. Methods of Treatment

The present invention provides for both prophylactic and therapeutic methods of treating a subject at risk of (or susceptible to) or having a cardiovascular disorder, e.g., a disorder associated with aberrant or unwanted HEAT expression or activity (e.g., hypertension or atherosclerosis). As used herein, “treatment” of a subject includes the application or administration of a therapeutic agent to a subject, or application or administration of a therapeutic agent to a cell or tissue from a subject, who has a diseases or disorder, has a symptom of a disease or disorder, or is at risk of (or susceptible to) a disease or disorder, with the purpose of curing, healing, alleviating, relieving, altering, remedying, ameliorating, improving, or affecting the disease or disorder, the symptom of the disease or disorder, or the risk of (or susceptibility to) the disease or disorder. As used herein, a “therapeutic agent” includes, but is not limited to, small molecules, peptides, polypeptides, antibodies, ribozymes, and antisense oligonucleotides.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the HEAT molecules of the present invention or HEAT modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant or unwanted HEAT expression or activity, by administering to the subject a HEAT or an agent which modulates HEAT expression or at least one HEAT activity. Subjects at risk for a disease which is caused or contributed to by aberrant or unwanted HEAT expression or activity can be identified by, for example, any or a combination of diagnostic or prognostic assays as described herein. Administration of a prophylactic agent can occur prior to the manifestation of symptoms characteristic of the HEAT aberrancy, such that a disease or disorder is prevented or, alternatively, delayed in its progression. Depending on the type of HEAT aberrancy, for example, a HEAT molecule, HEAT agonist or HEAT antagonist agent can be used for treating the subject. The appropriate agent can be determined based on screening assays described herein.

2. Therapeutic Methods

Another aspect of the invention pertains to methods of modulating HEAT expression or activity for therapeutic purposes. Accordingly, in an exemplary embodiment, the modulatory method of the invention involves contacting a cell capable of expressing HEAT with an agent that modulates one or more of the activities of HEAT protein activity associated with the cell, such that HEAT activity in the cell is modulated. An agent that modulates HEAT protein activity can be an agent as described herein, such as a nucleic acid or a protein, a naturally-occurring target molecule of a HEAT protein (e.g., a HEAT substrate), a HEAT antibody, a HEAT agonist or antagonist, a peptidomimetic of a HEAT agonist or antagonist, or other small molecule. In one embodiment, the agent stimulates one or more HEAT activities. Examples of such stimulatory agents include active HEAT protein and a nucleic acid molecule encoding HEAT that has been introduced into the cell. In another embodiment, the agent inhibits one or more HEAT activities. Examples of such inhibitory agents include antisense HEAT nucleic acid molecules, anti-HEAT antibodies, and HEAT inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a HEAT protein or nucleic acid molecule. In one embodiment, the method involves administering an agent (e.g., an agent identified by a screening assay described herein), or combination of agents that modulates (e.g., upregulates or downregulates) HEAT expression or activity. In another embodiment, the method involves administering a HEAT protein or nucleic acid molecule as therapy to compensate for reduced, aberrant, or unwanted HEAT expression or activity.

Stimulation of HEAT activity is desirable in situations in which HEAT is abnormally downregulated and/or in which increased HEAT activity is likely to have a beneficial effect. For example, stimulation of HEAT activity is desirable in situations in which a HEAT is downregulated and/or in which increased HEAT activity is likely to have a beneficial effect. Likewise, inhibition of HEAT activity is desirable in situations in which HEAT is abnormally upregulated and/or in which decreased HEAT activity is likely to have a beneficial effect.

3. Pharmacogenomics

The HEAT molecules of the present invention, as well as agents, or modulators which have a stimulatory or inhibitory effect on HEAT activity (e.g., HEAT gene expression) as identified by a screening assay described herein can be administered to individuals to treat (prophylactically or therapeutically) cardiovascular disorders (e.g., disorders characterized by aberrant modulation of cellular signaling, Ca²⁺ transport, gene expression, vascular smooth muscle tone, angiogenesis, and/or cell growth or proliferation mechanisms) associated with aberrant or unwanted HEAT activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a HEAT molecule or HEAT modulator as well as tailoring the dosage and/or therapeutic regimen of treatment with a HEAT molecule or HEAT modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate transporter deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug's target is known (e.g., a HEAT protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-transporter 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a HEAT molecule or HEAT modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a HEAT molecule or HEAT modulator, such as a modulator identified by one of the exemplary screening assays described herein.

4. Use of HEAT Molecules as Surrogate Markers

The HEAT molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the HEAT molecules of the invention may be detected, and may be correlated with one or more biological states in vivo. For example, the HEAT molecules of the invention may serve as surrogate markers for one or more disorders or disease states or for conditions leading up to disease states.

As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the causation of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35:258-264; and James (1994) AIDS Treatment News Archive 209.

The HEAT molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a HEAT marker) transcription or expression, the amplified marker may be in a quantity which is more readily detectable than the drug itself. Also, the marker may be more easily detected due to the nature of the marker itself; for example, using the methods described herein, anti-HEAT antibodies may be employed in an immune-based detection system for a HEAT protein marker, or HEAT-specific radiolabeled probes may be used to detect a HEAT mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al., U.S. Pat. No. 6,033,862; Hattis et al. (1991) Env. Health Perspect. 90:229-238; Schentag (1999) Am. J. Health-Syst. Pharm. 56 Suppl. 3:S21-S24; and Nicolau (1999) Am. J. Health-Syst. Pharm. 56 Suppl. 3:S16-S20.

The HEAT molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12):1650-1652). The presence or quantity of the pharmacogenomic marker is related to the predicted response of the subject in a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., HEAT protein or RNA) for specific tumor markers in a subject, a drug or course of treatment may be selected that is optimized for the treatment of the specific tumor likely to be present in the subject. Similarly, the presence or absence of a specific sequence mutation in HEAT DNA may correlate HEAT drug response. The use of pharmacogenomic markers therefore permits the application of the most appropriate treatment for each subject without having to administer the therapy.

E. Electronic Apparatus Readable Media and Arrays

Electronic apparatus readable media comprising HEAT sequence information is also provided. As used herein, “HEAT sequence information” refers to any nucleotide and/or amino acid sequence information particular to the HEAT molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” said HEAT sequence information includes detection of the presence or absence of a sequence (e.g., detection of expression of a sequence, fragment, polymorphism, etc.), determination of the level of a sequence (e.g., detection of a level of expression, for example, a quantitative detection), detection of a reactivity to a sequence (e.g., detection of protein expression and/or levels, for example, using a sequence-specific antibody), and the like. As used herein, “electronic apparatus readable media” refers to any suitable medium for storing, holding, or containing data or information that can be read and accessed directly by an electronic apparatus. Such media can include, but are not limited to: magnetic storage media, such as floppy discs, hard disc storage medium, and magnetic tape; optical storage media such as compact discs; electronic storage media such as RAM, ROM, EPROM, EEPROM and the like; and general hard disks and hybrids of these categories such as magnetic/optical storage media. The medium is adapted or configured for having recorded thereon HEAT sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatuses; networks, including a local area network (LAN), a wide area network (WAN) Internet, Intranet, and Extranet; electronic appliances such as a personal digital assistants (PDAs), cellular phone, pager and the like: and local and distributed processing systems.

As used herein, “recorded” refers to a process for storing or encoding information on the electronic apparatus readable medium. Those skilled in the art can readily adopt any of the presently known methods for recording information on known media to generate manufactures comprising the HEAT sequence information. A variety of software programs and formats can be used to store the sequence information on the electronic apparatus readable medium. For example, the sequence information can be represented in a word processing text file, formatted in commercially-available software such as WordPerfect and Microsoft Word, represented in the form of an ASCII file, or stored in a database application, such as DB2, Sybase, Oracle, or the like, as well as in other forms. Any number of dataprocessor structuring formats (e.g, text file or database) may be employed in order to obtain or create a medium having recorded thereon the HEAT sequence information.

By providing HEAT sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a cardiovascular disease or disorder or a pre-disposition to a cardiovascular disease or disorder, wherein the method comprises the steps of determining HEAT sequence information associated with the subject and based on the HEAT sequence information, determining whether the subject has a cardiovascular disease or disorder or a pre-disposition to a cardiovascular disease or disorder, and/or recommending a particular treatment for the disease, disorder, or pre-disease condition.

The present invention further provides in an electronic system and/or in a network, a method for determining whether a subject has a cardiovascular disease or disorder or a pre-disposition to a disease associated with HEAT wherein the method comprises the steps of determining HEAT sequence information associated with the subject, and based on the HEAT sequence information, determining whether the subject has a cardiovascular disease or disorder or a pre-disposition to a cardiovascular disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has a cardiovascular disease or disorder or a pre-disposition to a cardiovascular disease or disorder associated with HEAT, said method comprising the steps of receiving HEAT sequence information from the subject and/or information related thereto, receiving phenotypic information associated with the subject, acquiring information from the network corresponding to HEAT and/or a cardiovascular disease or disorder, and based on one or more of the phenotypic information, the HEAT information (e.g., sequence information and/or information related thereto), and the acquired information, determining whether the subject has a cardiovascular disease or disorder or a pre-disposition to a cardiovascular disease or disorder. The method may further comprise the step of recommending a particular treatment for the disease, disorder or pre-disease condition.

The present invention also provides a business method for determining whether a subject has a cardiovascular disease or disorder or a pre-disposition to a cardiovascular disease or disorder, said method comprising the steps of receiving information related to HEAT (e.g., sequence information and/or information related thereto), receiving phenotypic information associated with the subject, acquiring information from the network related to HEAT and/or related to a cardiovascular disease or disorder, and based on one or more of the phenotypic information, the HEAT information, and the acquired information, determining whether the subject has a cardiovascular disease or disorder or a pre-disposition to a cardiovascular disease or disorder. The method may further comprise the step of recommending a particular treatment for the disease, disorder or pre-disease condition.

The invention also includes an array comprising a HEAT sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be HEAT. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a cardiovascular disease or disorder, progression of cardiovascular disease or disorder, and processes, such a cellular transformation associated with the cardiovascular disease or disorder.

The array is also useful for ascertaining the effect of the expression of a gene on the expression of other genes in the same cell or in different cells (e.g., ascertaining the effect of HEAT expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including HEAT) that could serve as a molecular target for diagnosis or therapeutic intervention.

This invention is further illustrated by the following examples which should not be construed as limiting. The contents of all references, patents and published patent applications cited throughout this application, as well as the sequence listing and the figures, are incorporated herein by reference.

### EXAMPLES

### Example 1

### Identification and Characterization of Human Heat cDNA

In this example, the identification and characterization of the genes encoding human HEAT-1 (clone 49937), human HEAT-2 (clone 49931), and human HEAT-3 (clone 49933) is described.

Isolation of the Human HEAT cDNAs

The invention is based, at least in part, on the discovery of genes encoding novel members of the E1-E2 ATPase family. The entire sequence of human clones Fbh49937, Fbh49931, and Fbh49933 were determined and found to contain open reading frames termed human “HEAT-1,” human “HEAT-2,” and human “HEAT-3,” respectively.

The nucleotide sequence encoding the human HEAT-1 is shown in FIGS. 1A-1D and is set forth as SEQ ID NO:1. The protein encoded by this nucleic acid molecule comprises about 1180 amino acids and has the amino acid sequence shown in FIGS. 1A-1D and set forth as SEQ ID NO:2. The coding region (open reading frame) of SEQ ID NO:1 is set forth as SEQ ID NO:3. Clone Fbh49937, comprising the coding region of human HEAT-1, was deposited with the American Type Culture Collection (ATCC®), 10801 University Boulevard, Manassas, Va. 20110-2209, on ______, and assigned Accession No. ______.

The nucleotide sequence encoding the human HEAT-2 is shown in FIGS. 11A-11E and is set forth as SEQ ID NO:5. The protein encoded by this nucleic acid molecule comprises about 1256 amino acids and has the amino acid sequence shown in FIGS. 11A-11E and set forth as SEQ ID NO:6. The coding region (open reading frame) of SEQ ID NO:1 is set forth as SEQ ID NO:7. Clone Fbh49931, comprising the coding region of human HEAT-2, was deposited with the American Type Culture Collection (ATCC®), 10801 University Boulevard, Manassas, Va. 20110-2209, on ______, and assigned Accession No. ______.

The nucleotide sequence encoding the human HEAT-3 is shown in FIGS. 22A-22D and is set forth as SEQ ID NO:8. The protein encoded by this nucleic acid molecule comprises about 1204 amino acids and has the amino acid sequence shown in FIGS. 22A-22D and set forth as SEQ ID NO:9. The coding region (open reading frame) of SEQ ID NO:8 is set forth as SEQ ID NO:10. Clone Fbh49933, comprising the coding region of human HEAT-3, was deposited with the American Type Culture Collection (ATCC®), 10801 University Boulevard, Manassas, Va. 20110-2209, on ______, and assigned Accession No. ______.

Analysis of the Human HEAT Molecules

The amino acid sequences of human HEAT-1, HEAT-2, and HEAT-3 were analyzed using the program PSORT (available online; see Nakai, K. and Kanehisa, M. (1992) Genomics 14:897-911) to predict the localization of the proteins within the cell. This program assesses the presence of different targeting and localization amino acid sequences within the query sequence. The results of the analyses show that human HEAT-1 may be localized to the endoplasmic reticulum, milochondria, secretory vesicles, or vacuoles. The results of these analyses further show that human HEAT-2 may be localized to the endoplasmic reticulum or the mitochondria and that human HEAT-3 may be localized to endoplasmic reticulum, the mitochondria or vacuoles.

Analyses of the amino acid sequence of human HEAT-1, HEAT-2, and HEAT-3 were performed using MEMSAT. These analyses resulted in the identification of twelve possible transmembrane domains in the amino acid sequence of human HEAT-2 at residues 29-50, 211-227, 234-253, 294-317, 410-434, 449-469, 941-960, 968-985, 1000-1020, 1076-1092, 1105-1129, and 1144-1160 of SEQ ID NO:6 (FIGS. 13 and 14). These analyses further resulted in the identification of twelve possible transmembrane domains in the amino acid sequence of human HEAT-3 at residues 65-89, 99-116, 242-258, 265-281, 445-464, 493-509, 990-1007, 1015-1031, 1049-1073, 1049-1073, 1103-1119, 1134-1151, and 1171-1187 of SEQ ID NO:9 (FIGS. 24 and 25). The analysis of human HEAT-1 predicted twelve possible transmembrane domains in the amino acid sequence of human HEAT-1 (SEQ ID NO:2) at about residues 8-25, 47-65, 256-276, 428-448, 464-484, 900-920, 936-954, 963-987, 994-1015, 1049-1065, 1079-1102, and 1118-1134. The potential transmembrane domain at about residues 900-920 has a notably low score of only 0.4 by MEMSAT analysis. Further analysis of the amino acid sequence of SEQ ID NO:2 (e.g., alignment with, for example, a known C. elegans E1-E2 ATPase cation transporter) resulted in the identification of a twelfth transmembrane domain at about amino acid residues 231-253 of SEQ ID NO:2. Accordingly, the human HEAT-1 protein of SEQ ID NO:2 is predicted to have at least twelve transmembrane domains, for example, at about residues 8-25, 47-65, 231-253, 256-276, 428-448, 464-484, 936-954, 963-987, 994-1015, 1049-1065, 1079-1102, and 1118-1134.

Searches of the amino acid sequences of human HEAT-1, HEAT-2, and HEAT-3 were also performed against the HMM database (FIGS. 3, 12, and 23, respectively). These searches resulted in the identification of an “E1-E2 ATPase” domain in the amino acid sequence of HEAT-1 at about residues 299-387 (score=51.4) of SEQ ID NO:2 (FIG. 3). These searches also resulted in the identification of an “E1-E2 ATPase” domain in the amino acid sequence of human HEAT-2 at about residues 278-365 (score=53.4) of SEQ ID NO:6. These searches further resulted in the identification of an “E1-E2 ATPase” domain in the amino acid sequence of human HEAT-3 at about residues 302-392 (score=37.0) of SEQ ID NO:9.

Searches of the amino acid sequence of human HEAT-1 were performed against the Prosite database. These searches resulted in the identification of an “E1-E2 ATPases phosphorylation site” at about residues 513-519 of SEQ ID NO:2. These searches also resulted in the identification in the amino acid sequence of human HEAT-1 of a number of potential N-glycosylation sites, cAMP- and cGMP-dependent protein kinase phosphorylation sites, protein kinase C phosphorylatiou sites, casein kinase II phosphorylation sites, and N-myristoylation sites.

Searches of the amino acid sequence of human HEAT-2 were also performed against the Prosite database. These searches resulted in the identification of an “E1-E2 ATPases phosphorylation site” at about residues 498-504 of SEQ ID NO:6 (FIGS. 14A-14B). These searches also resulted in the identification in the amino acid sequence of human HEAT-2 of a number of potential N-glycosylation sites, cAMP- and cGMP-dependent protein kinase phosphorylation sites, protein kinase C phosphorylation sites, casein kinase II phosphorylation sites, tyrosine phosphorylation sites, and N-myristoylation sites.

Searches of the amino acid sequence of human HEAT-3 were further performed against the Prosite database. These searches resulted in the identification of an “E1-E2 ATPases phosphorylation site” at about residues 533-539 of SEQ ID NO:9 (FIGS. 25A-25B). These searches also resulted in the identification in the amino acid sequence of human HEAT-3 of a number of potential N-glycosylation sites, cAMP- and cGMP-dependent protein kinase phosphorylation sites, protein kinase C phosphorylation sites, casein kinase II phosphorylation sites, and N-myristoylation sites.

The amino acid sequence of human HEAT-2 was used as a database query using the BLASTP program. This search established that human HEAT-2 has the highest homology to a putative yeast Ca²⁺ -transporting ATPase (high score=798, probability=2.9e-87).

Tissue Expression Analysis of HEAT-1, HEAT-2, and HEAT-3 mRNA Using Transcriptional Profiling and Tagman Analysis

This example describes the tissue distribution of human HEAT-2 mRNA, as determined using transcriptional profiling analysis and the TaqMan™ procedure. For transcriptional profiling analysis, an array of several thousand cDNA clones are spotted onto a nylon membrane and probed with a complex probe prepared by radiolabeling cDNA made from mRNA from, for example, normal tissue, and another, separate probe made from mRNA from another tissue, for example, diseased tissue. Expression levels of each gene in the first (e.g., normal) and the second (e.g., diseased) tissue are then compared. Transcriptional profiling thus allows assessment of the expression level of several thousand genes in an mRNA sample at the same time.

The Taqman™ procedure is a quantitative, reverse transcription PCR-based approach for detecting mRNA. The RT-PCR reaction exploits the 5′ nuclease activity of AmpliTaq GoId™ DNA Polymerase to cleave a TaqMan™ probe during PCR. Briefly, cDNA is generated from the samples of interest and used as the starting material for PCR amplification. In addition to the 5′ and 3′ gene-specific primers, a gene-specific oligonucleotide probe (complementary to the region being amplified) is included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluoresceinl), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

Endothelial Cell Paradigms

To induce tube formation, human microvascular endothelial cells isolated from the lung (HMVECs) were plated on Matrigel to induce capillary-like tube formation. At 5 hours, the cells were actively forming tubes, and RNA was harvested. Additional RNA samples were prepared from cells 25 hours after plating on Matrigel when tube formation was complete, and from actively proliferating and confluent HMVECs grown on plastic.

Cells were also treated with laminar shear stress (LSS) of 7 dyn/cm² for 24-30 hours, LSS plus one or six additional hours of 12 dyn/cm² (“1 h up” or “6 h up”), or LSS plus one or six additional hours of 2 dyn/cm² (“1 h down” or “6 h down”).

HEAT-1

The expression levels of human HEAT-1 mRNA in various human and monkey cell types and tissues was first determined using the Taqman procedure. As shown in FIG. 5, HEAT-1 is highly expressed in coronary artery vascular smooth muscle cells, prostate epithelial cells, pancreas, and brain (including cortex, hypothalamus, dorsal root ganglion cells, and glial cells/astrocytes).

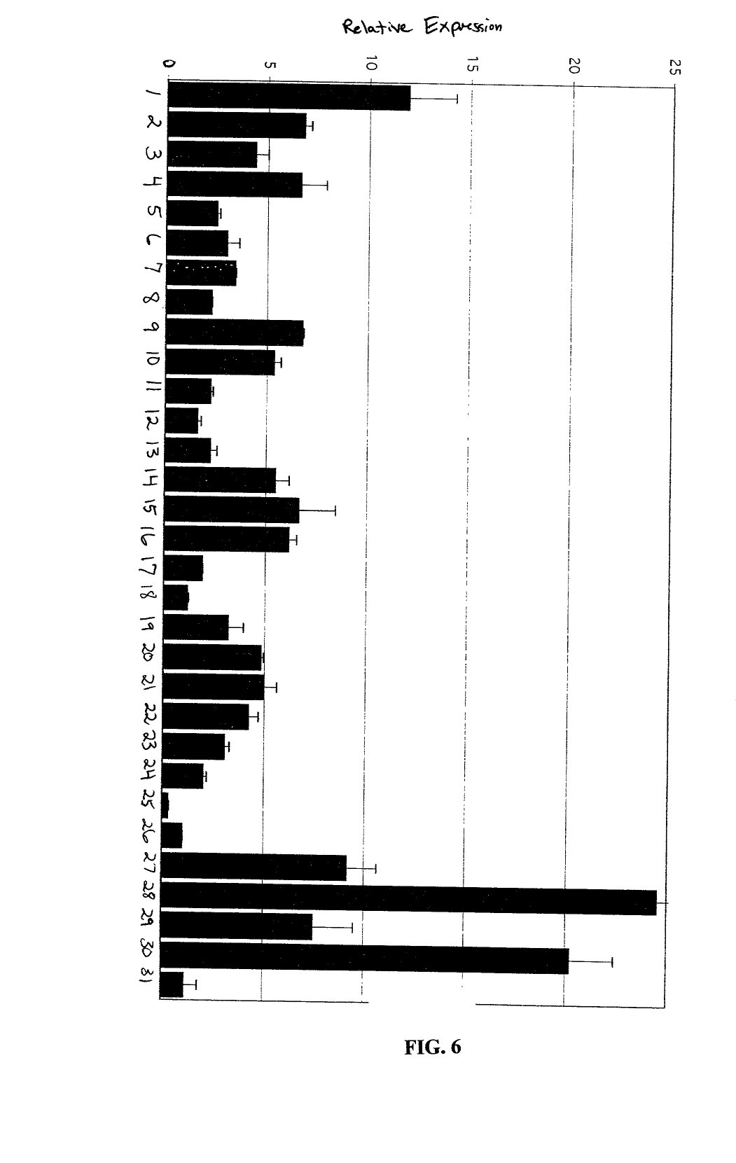

The expression levels of human HEAT-1 mRNA in various human vascular rich organs was then determined using the Taqman procedure. As shown in FIG. 6, HEAT-1 is highly expressed in Wilms' tumor, normal spinal cord, and microvascular endothelial cells.

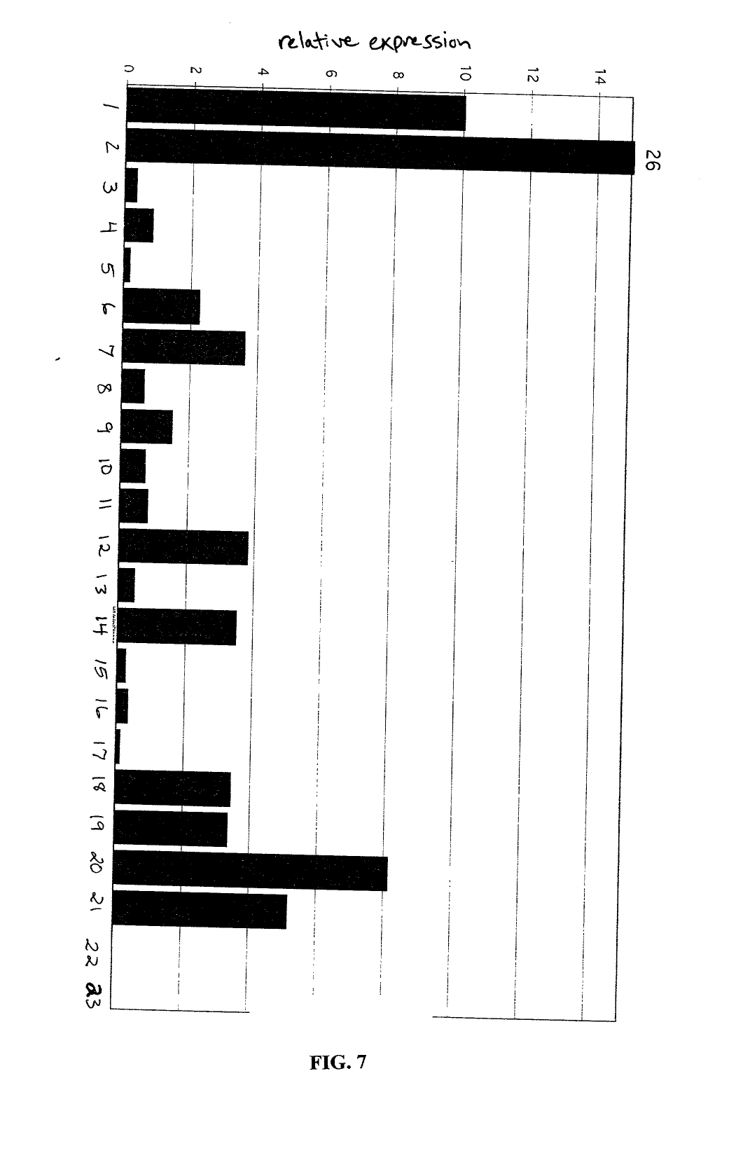

In another experiment, the expression levels of human HEAT-1 mRNA in various human and monkey vessels was determined using the Taqman procedure. As shown in FIG. 7, HEAT-1 is highly expressed in vessels such as arteries and veins.

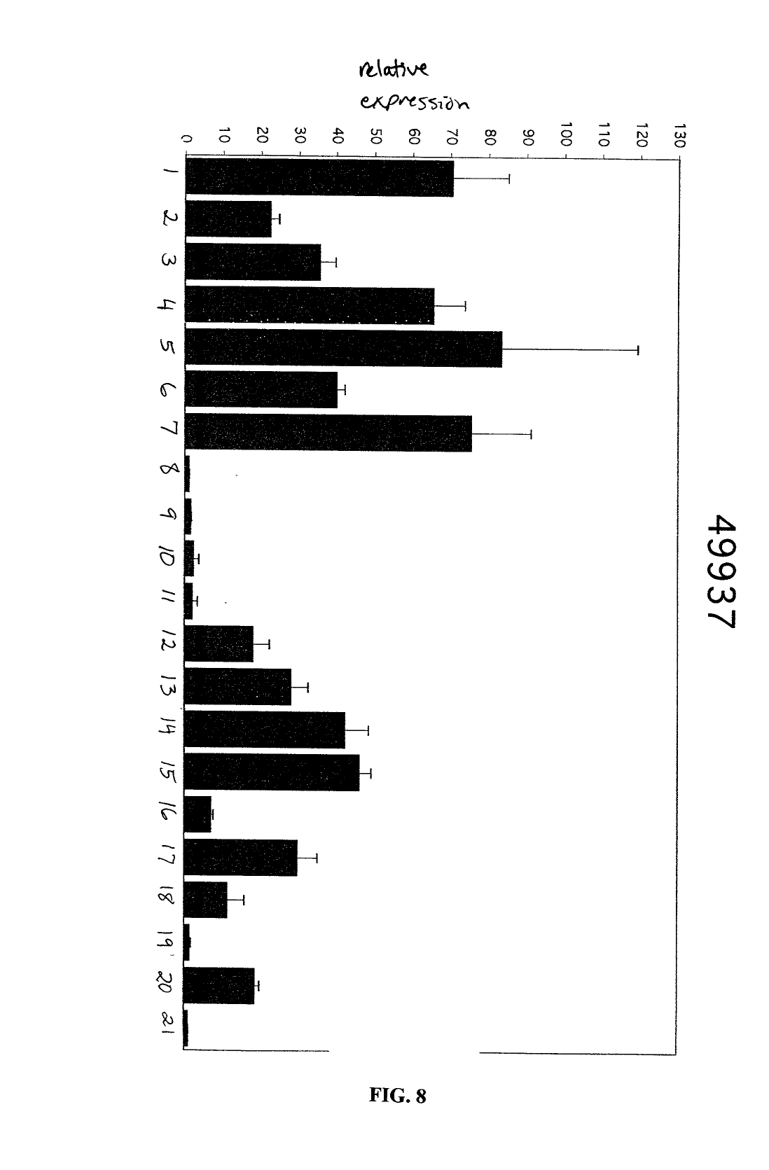

The expression levels of human HEAT-1 mRNA in various human coronary vascular cell types was also determined using the Taqman procedure. As shown in FIG. 8, HEAT-1 is highly expressed in coronary and vascular smooth muscle cells, as compared to other cell types.

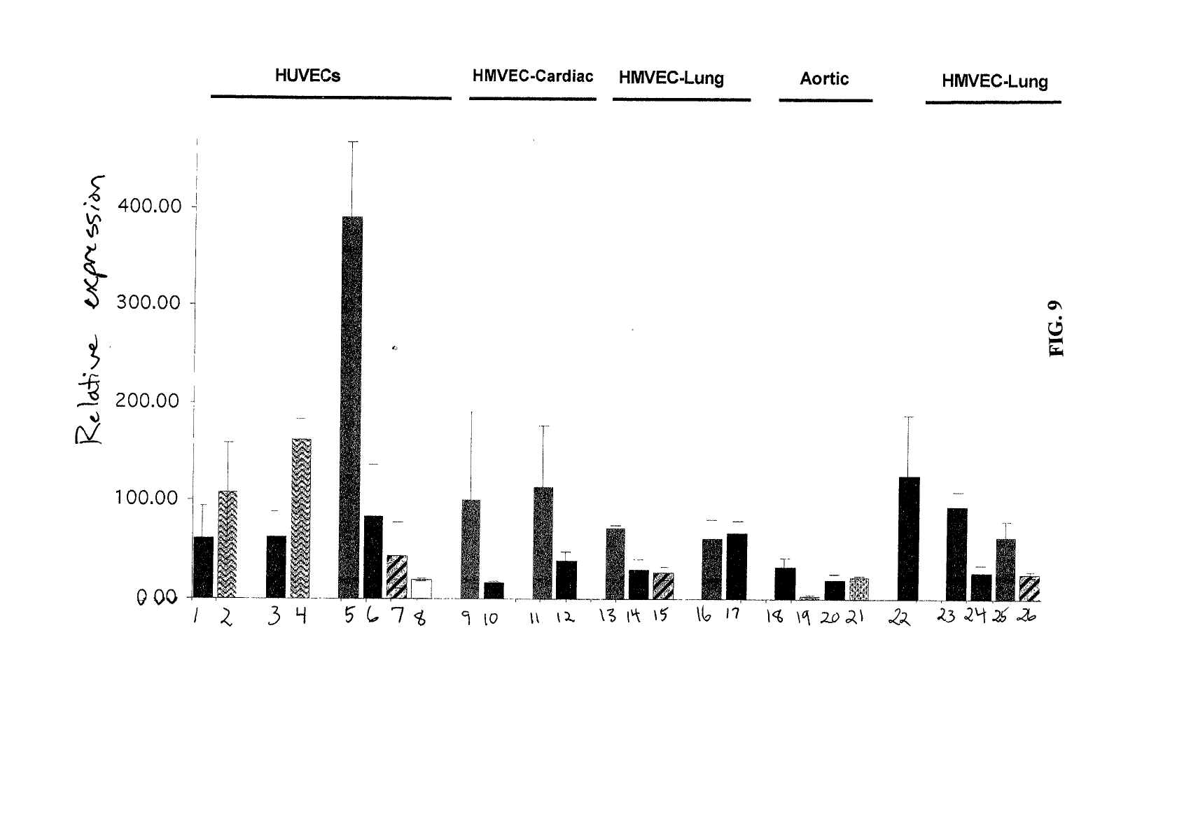

The expression levels of human HEAT-1 mRNA in various human endothelial cell paradigms was determined using the Taqman procedure. As shown in FIGS. 5, 9, and 10, human HEAT-1 is upregulated during shear stress of endothelial cells. As shown in FIG. 9, human HEAT-1 is upregulated during proliferation and tube formation of endothelial cells. These data strongly link human HEAT-1 to a role in angiogenesis.

HEAT-2

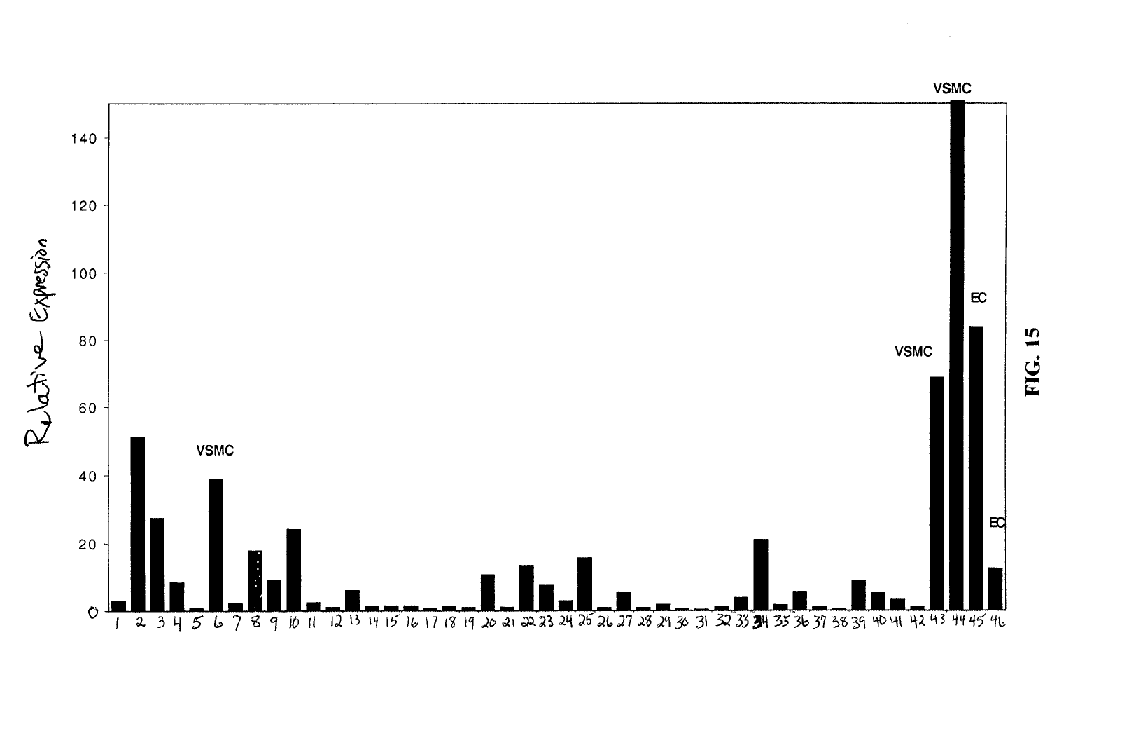

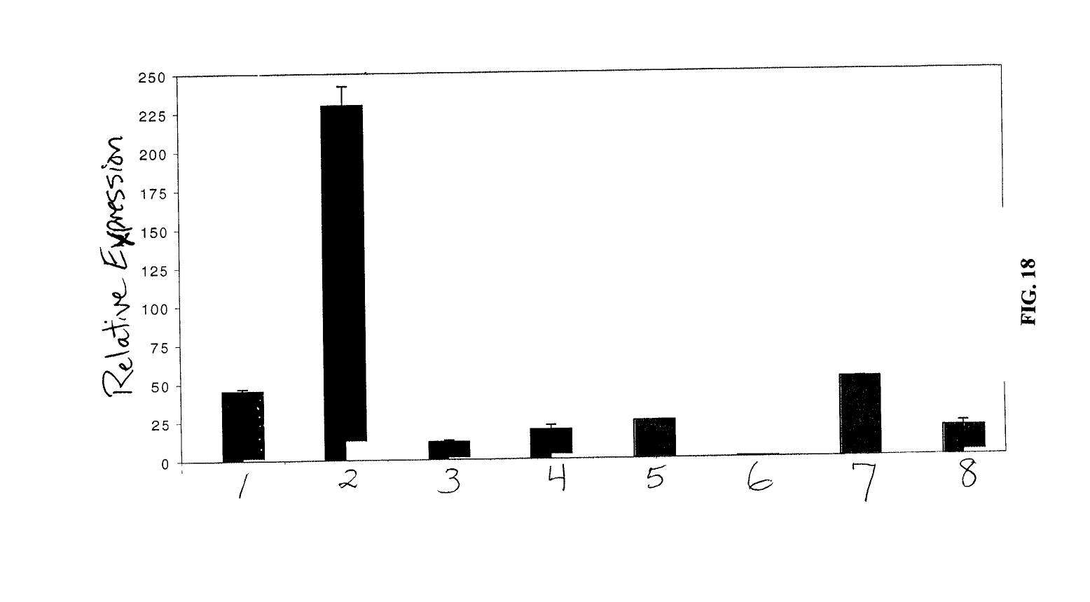

The expression levels of human HEAT-2 mRNA in various human and monkey cell types and tissues was first determined using transcriptional profiling. As shown in FIG. 18, HEAT-2 is highly expressed in coronary artery vascular smooth muscle cells, as compared to other tissues such as aortic vascular smooth muscle cells, umbilical vein endothelial cells, microvascular endothelial cells, heart, liver, aorta, and vein. The expression levels of human HEAT-2 mRNA in various human cell types and tissues was confirmed in a second experiment using the Taqman procedure (see FIG. 15).

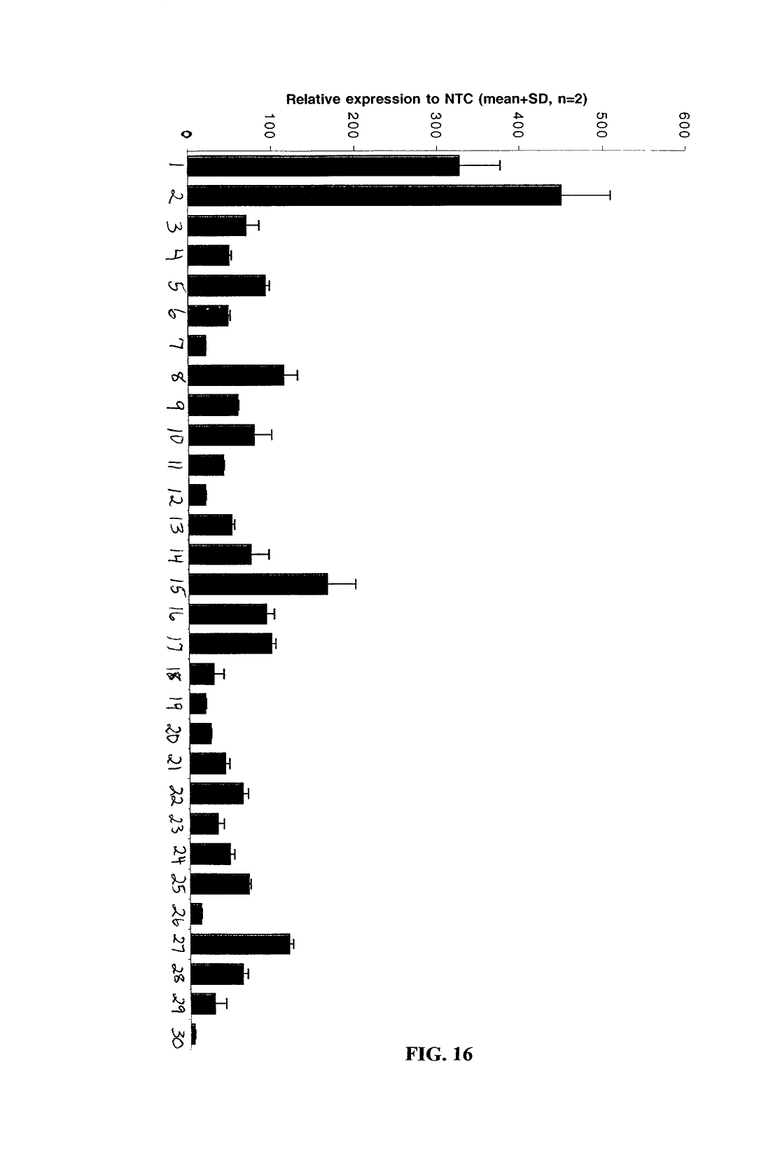

The expression levels of human HEAT-2 mRNA in various human vascular rich organs was then determined using the Taqman procedure. As shown in FIG. 16, HEAT-2 is highly expressed in the heart.

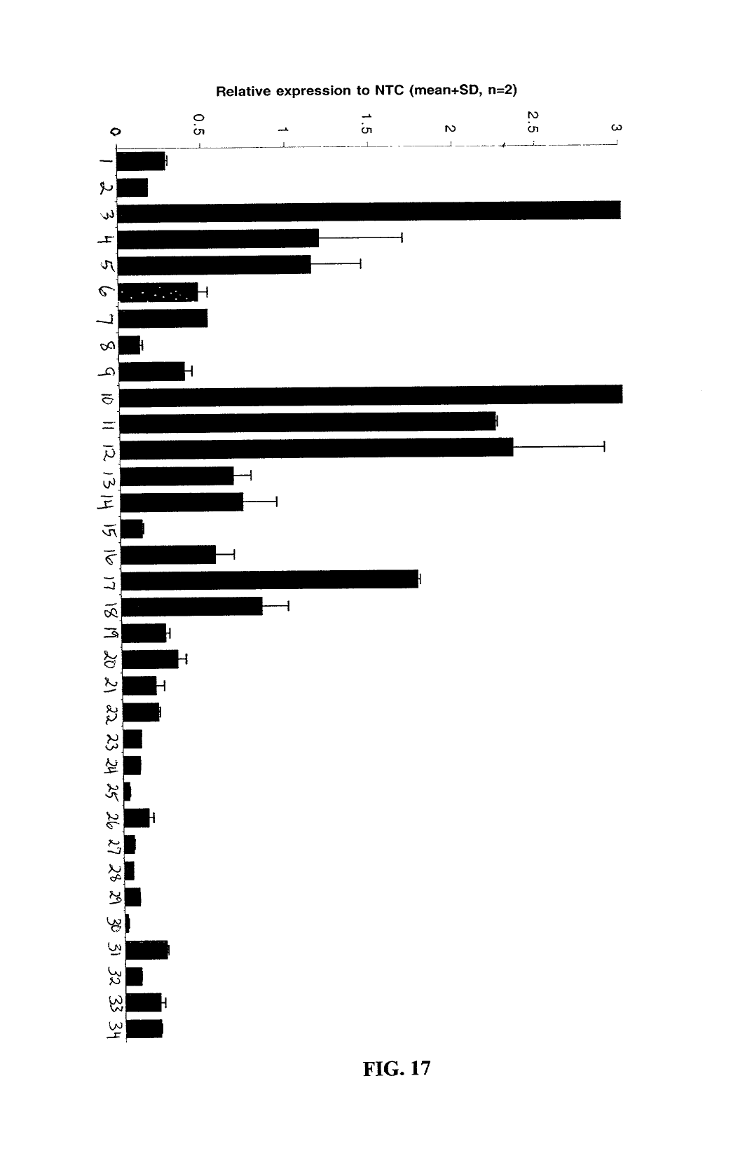

In another experiment, the expression levels of human HEAT-2 nRNA in various human and monkey vessels was determined using the Taqman procedure and in situ hybridization. As shown in FIG. 17, HEAT-2 is highly expressed in vessels such as arteries and veins.

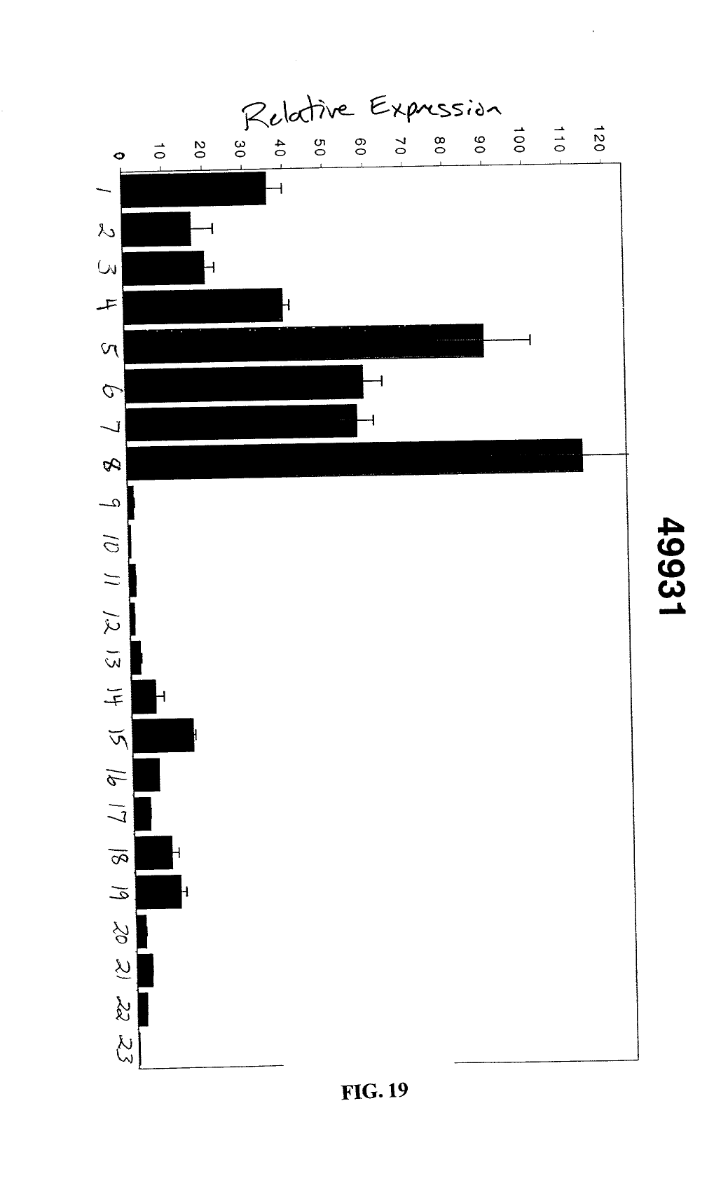

The expression levels of human HEAT-2 mRNA in various human coronary vascular cell types was also determined using the Taqman procedure. As shown in FIG. 19, HEAT-2 is highly expressed in coronary vascular smooth muscle cells, as compared to other cell types.

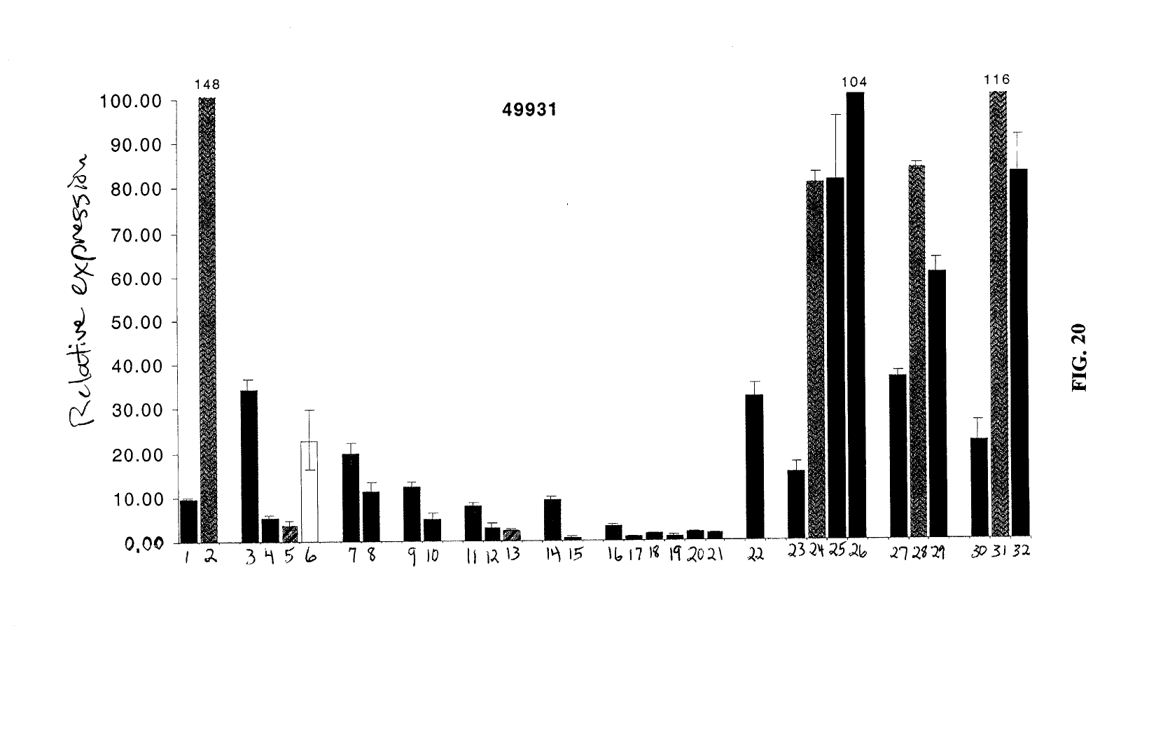

The expression levels of human HEAT-2 mRNA in various human endothelial cell paradigms was determined using the Taqman procedure. As shown in FIGS. 15 and 20, HEAT-2 is upregulated during shear and proliferation of endothelial cells. These data strongly link HEAT-2 to a role in angiogenesis.

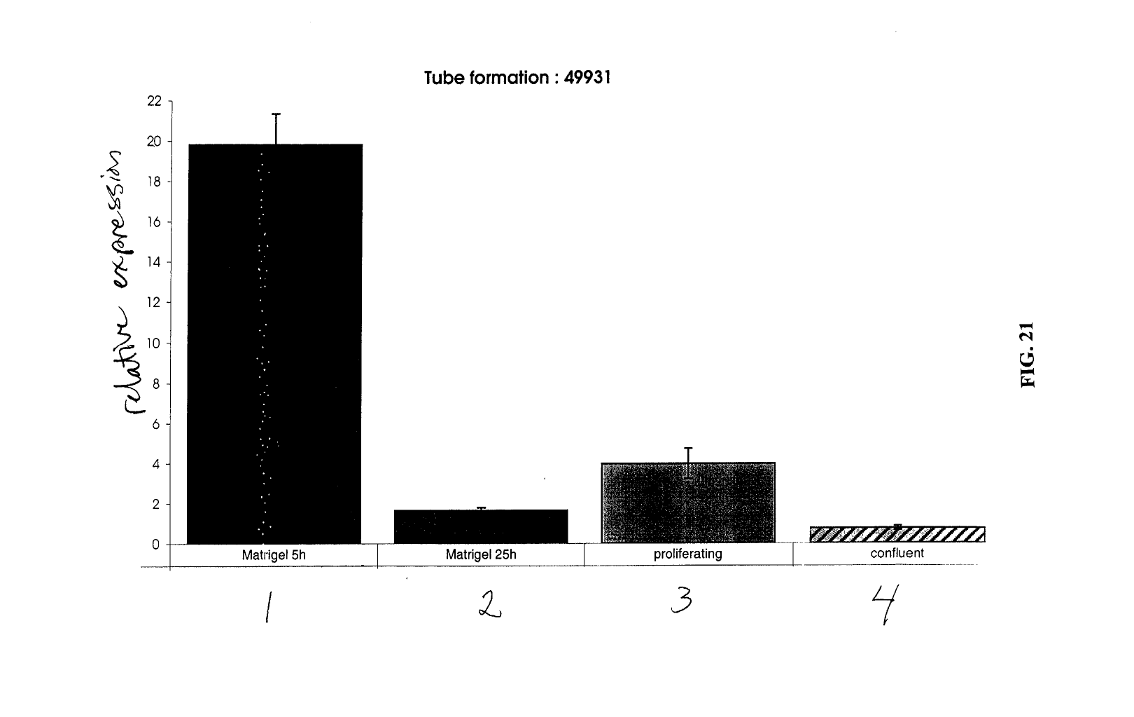

As shown in FIG. 21, human HEAT-2 is upregulated during tube formation of endothelial cells. The expression level of human HEAT-2 is 5-fold higher in the 5-hour Matrigel sample than in any other sample, indicating that expression is significantly induced during the process of capillary-like tube formation. There is also significantly higher expression in proliferating HMVECs than in confluent HMVECs grown on plastic. These results indicate a pro-angiogenic function for human HEAT-2.

Human HEAT-2 mRNA expression was also detected by in situ hybridization analysis in human endothelial cells and myocytes in the heart and in endothelial cells and inflammatory cells in ApoE knockout mouse diseased aortic roots.

HEAT-3

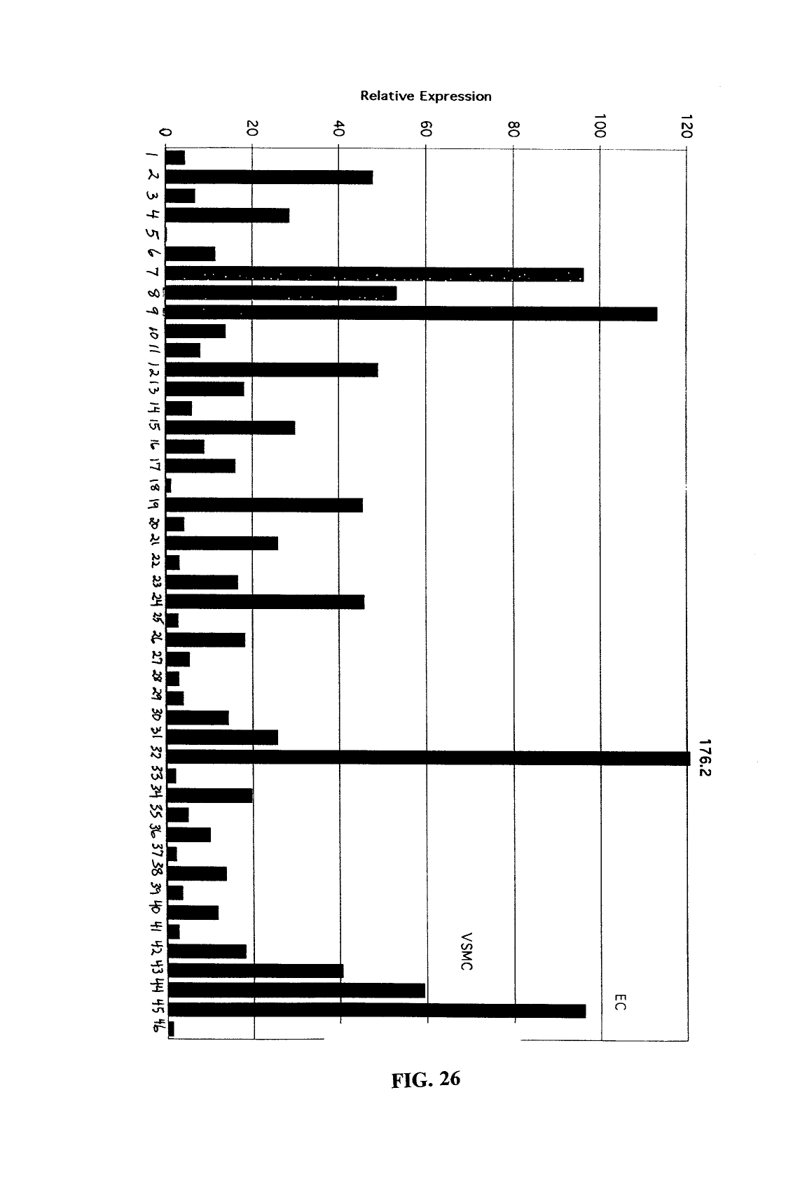

The expression levels of human HEAT-3 mRNA in various human and monkey cell types and tissues was first determined using the Taqman procedure. As shown in FIG. 26, HEAT-3 is highly expressed in coronary artery vascular smooth muscle cells, prostate epithelial cells, pancreas, and brain (including cortex, hypothalamus, and glial cells/astrocytes).

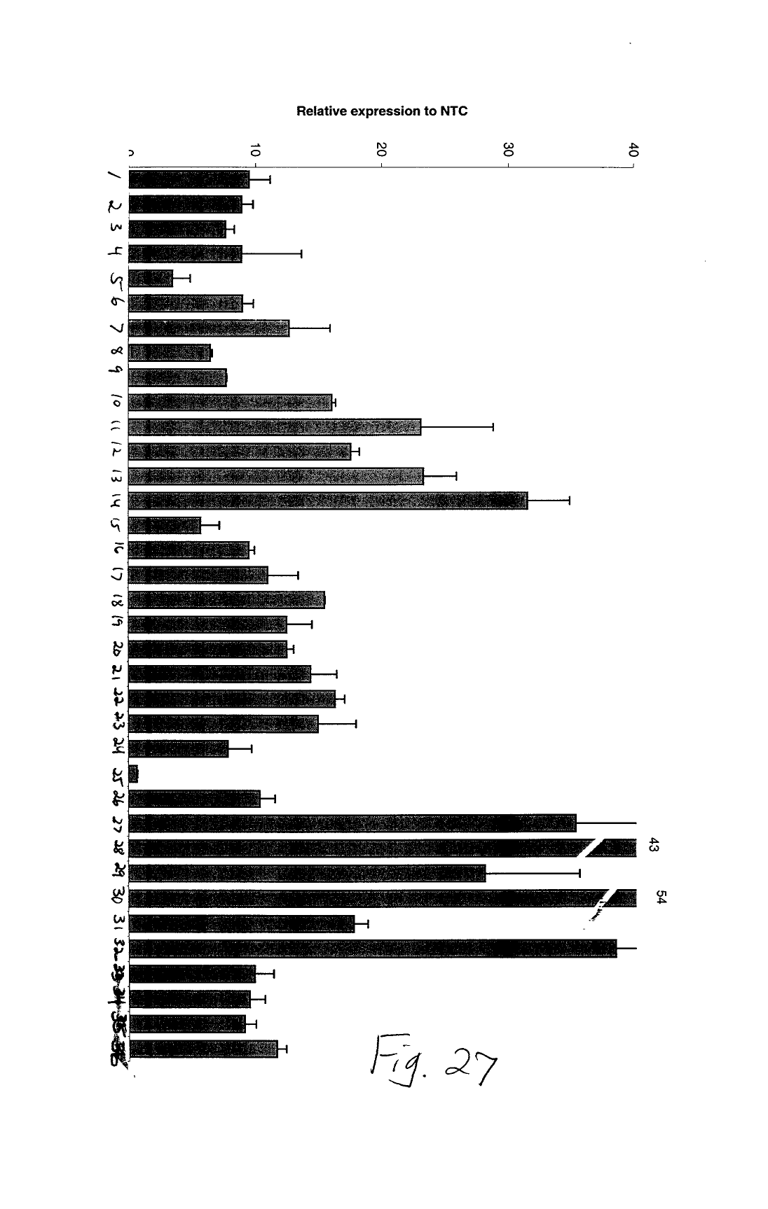

The expression levels of human HEAT-3 mRNA in various human vascular rich organs was then determined using the Taqman procedure. As shown in FIG. 27, HEAT-3 is expressed in the heart, kidney, and skeletal muscle.

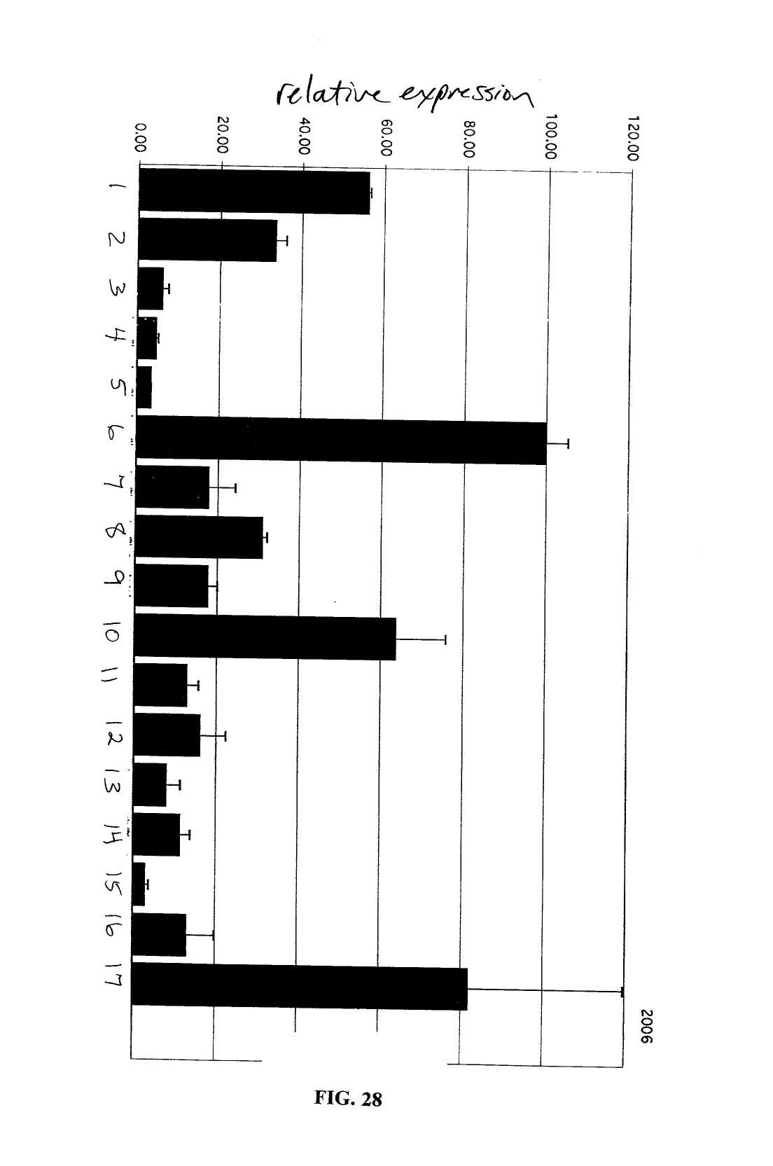

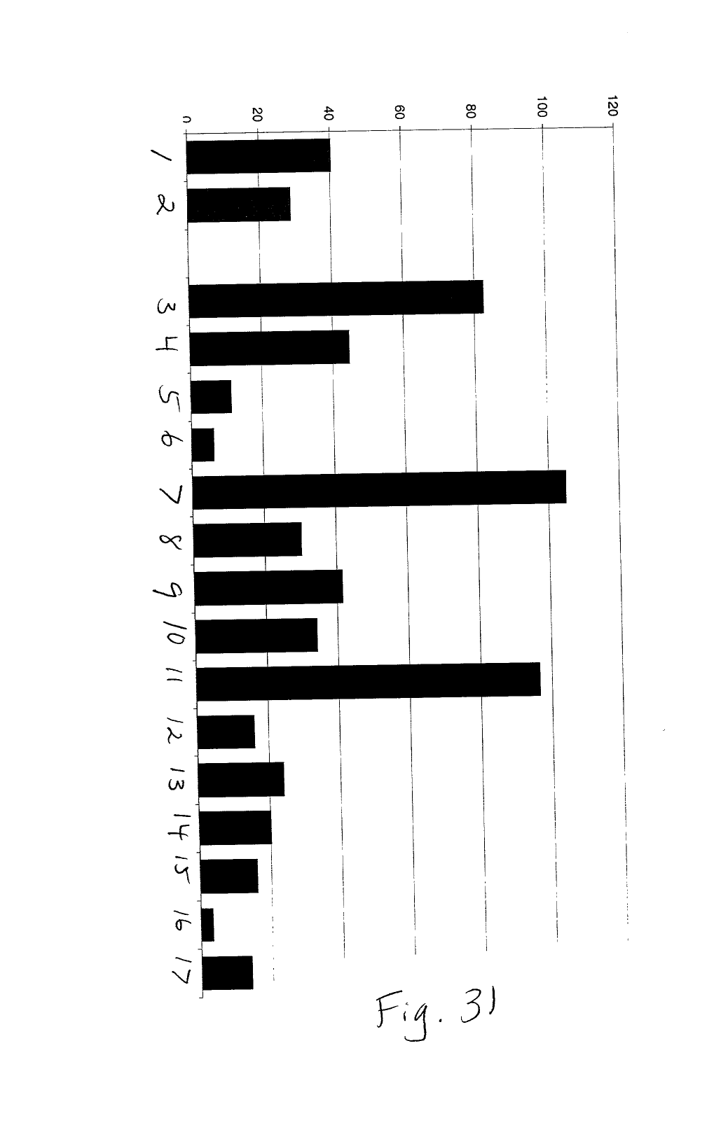

In another experiment, the expression levels of human HEAT-3 mRNA in various vessels was determined using the Taqman procedure. As shown in FIGS. 28 and 31, HEAT-3 is highly expressed in vessels such as arteries and veins.

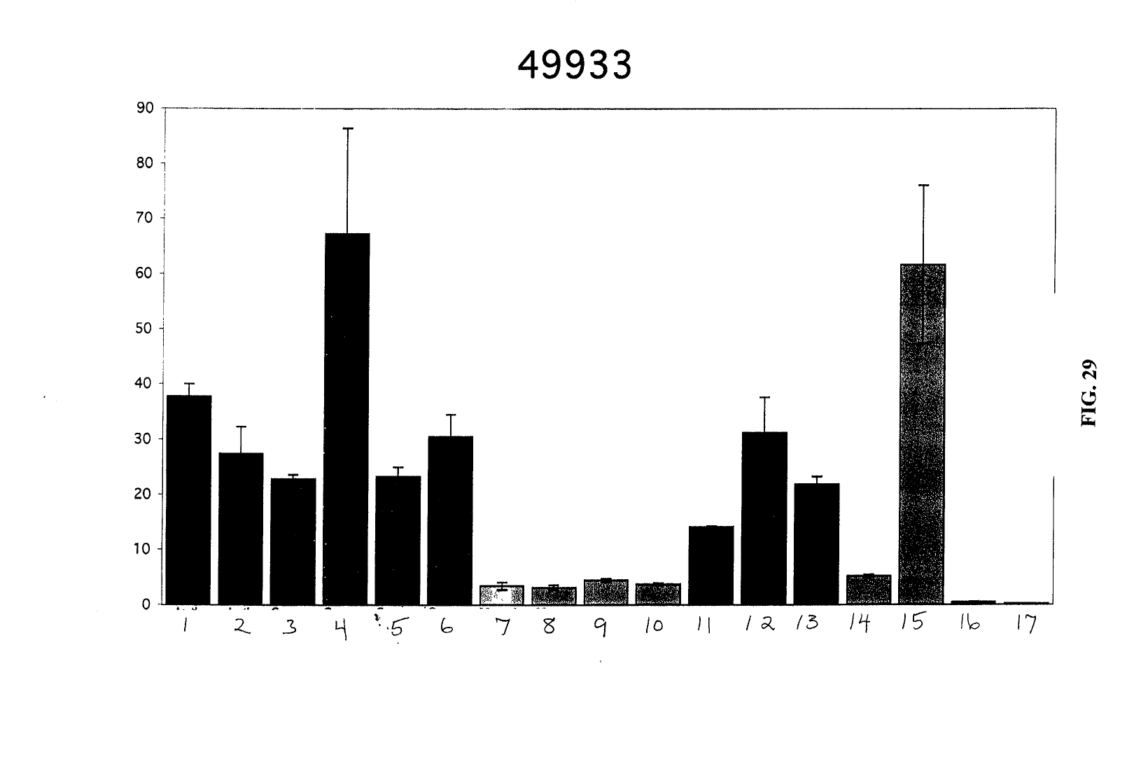

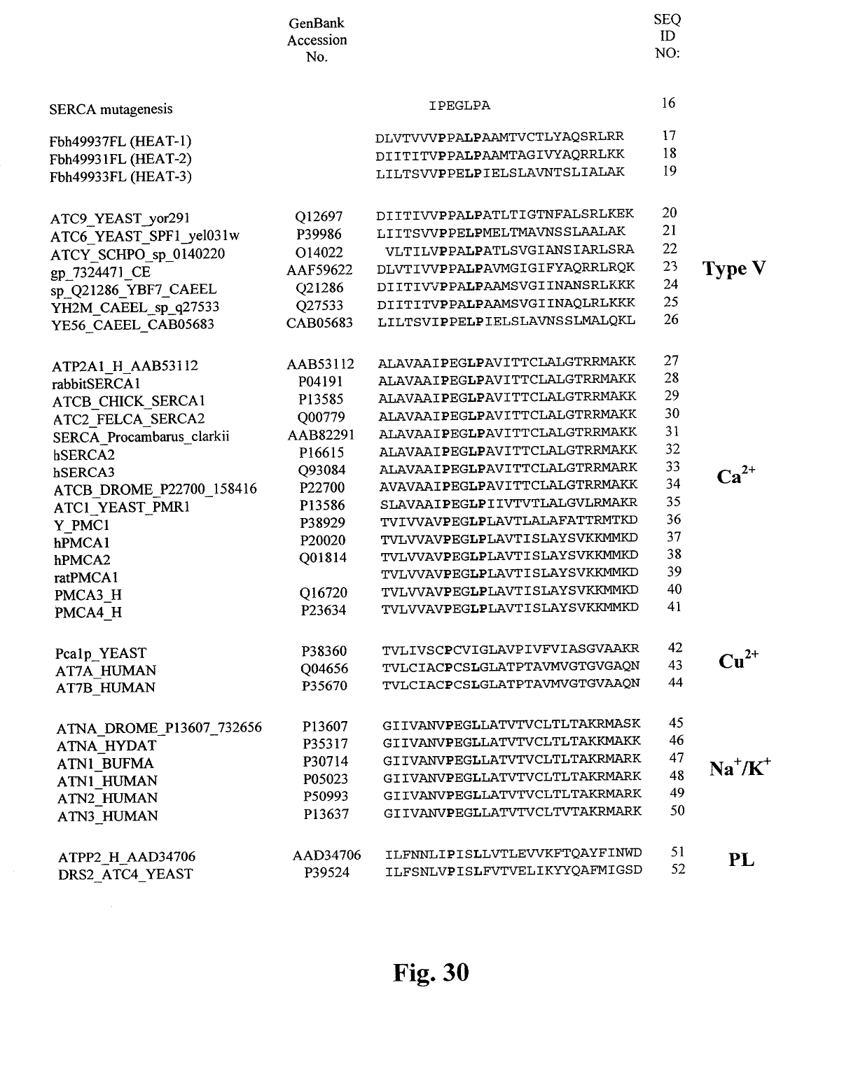

The expression levels of human HEAT-3 mRNA in various human coronary vascular cell types was also determined using the Taqman procedure. As shown in FIG. 29, HEAT-3 is highly expressed in coronary and aortic vascular smooth muscle ceils, as well as in renal proximal tubule epithelium, ts compared to other cell types.

Tissue Distribution of HEAT mRNA Using in situ Analysis

This example describes the tissue distribution of human HEAT mRNA, as was determined using in situ hybridization analysis. For in situ analysis, various tissues, e.g., vascular tissue or heart tissue, were first frozen on dry ice. Ten-micrometer-thick sections of the tissues were postfixed with 4% formaldehyde in DEPC-treated 1×phosphate-buffered saline at room temperature for 10 minutes before being rinsed twice in DEPC 1×phosphate-buffered saline and once in 0.1 M triethanolamine-HCl (pH 8.0). Following incubation in 0.25% acetic anhydride-0.1 M triethanolamine-HCl for 10 minutes, sections were rinsed in DEPC 2×SSC (1×SSC is 0.15 M NaCl plus 0.015 M sodium citrate). Tissue was then dehydrated through a series of ethanol washes, incubated in 100% chloroform for 5 minutes, and then rinsed in 100% ethanol for 1 minute and 95% ethanol for 1 minute and allowed to air dry.

Hybridizations were performed with ³⁵S-radiolabeled (5×10⁷ cpm/ml) cRNA probes. Probes were incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1×, 1×Denhardt's solution, 50% formamide, 10% dextran sulfate, 100 mM dithiothreitol, 0.1% sodium dodecyl sulfate (SDS), and 0. 1% sodium thiosulfate for 18 hours at 55° C.

After hybridization, slides were washed with 2×SSC. Sections were then sequentially incubated at 37° C. in TNE (a solution containing 10 mM Tris-HCl (pH 7.6), 500 mM NaCl, and 1 mM EDTA), for 10 minutes, in TNE with 10 μg of RNase A per ml for 30 minutes, and finally in TNE for 10 minutes. Slides were then rinsed with 2×SSC at room temperature, washed with 2×SSC at 50° C. for 1 hour, washed with 0.2×SSC at 55° C. for 1 hour, and 0.2×SSC at 60° C. for 1 hour. Sections were then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

Using in situ hybridization analysis, HEAT-2 mRNA was found to be expressed in human endothelial cells and myocytes in the heart and in endothelial cells and inflammatory cells in ApoE knockout mice diseased aortic roots.

### Example 2

### Expression of Recombinant HEAT Protein in Bacterial Cells

In this example, human HEAT is expressed as a recombinant glutathione-S-transferase (GST) fusion polypeptide in E. coli and the fusion polypeptide is isolated and characterized. Specifically, human HEAT is fused to GST and this fusion polypeptide is expressed in E. coli, e.g., strain PEB199. Expression of the GST-HEAT fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expression of Recombinant HEAT Protein in COS Cells

To express the human HEAT gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire HEAT protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the HEAT DNA sequence is amplified by PCR using two primers. The 5′ primer contains the restriction site of interest followed by approximately twenty nucleotides of the HEAT coding sequence starting from the initiation codon; the 3′ end sequence contains complementary sequences to the other restriction site of interest, a translation stop codon, the HA tag or FLAG tag and the last 20 nucleotides of the HEAT coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the HEAT gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the HEAT-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J. et al., Molecular Cloning: A Laboratory Manual 2^{nd} ed., Cold Spring Harbor Laboratory, Cold Spring Harbor Laboratory Press, Cold Spring Harbor, N.Y., 1989. The expression of the HEAT polypeptide is detected by radiolabeling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the HEAT coding sequence is cloned directly into the polylinker of the pCDNA/Amp vector using the appropriate restriction sites. The resulting plasmid is transfected into COS cells in the manner described above, and the expression of the HEAT polypeptide is detected by radiolabeling and immunoprecipitation using a HEAT specific monoclonal antibody.

### Example 4

### Assesment of Microvessel Contraction

This example describes the assessment of microvessel contraction using rat microvessels, as described in, for example, Bischoff, A. et al. (2000) Br. J. Pharmacol. 130:1871-1877. Microvessels (e.g., mesenteric or renal microvessels such as interlobar arteries) are prepared from rats (e.g., adult Wistar rats) as described in Chen et al. (1996) Naunyn-Schmiedeberg's Arch. Pharmacol. 353:314-323 and Chen et al. (1997) J. Auton. Pharmacol. 17:137-146. Rats are killed by either decapitation or an overdose of thiobutabarbitone. The vessels are mounted on 40 μm diameter stainless steel wires in a myograph chamber for isometric recording of tension development. The vessels are then bathed in Krebs-Henseleit buffer of the following composition: 119 mM NaCl, 25 mM NaHCO₃, 4.7 mM KCl, 1.18 mM KH₂PO₄, 1.17 mM MgSO₄, 2.5 mM CaCl₂, 0.026 mM EDTA, and 5.5 mM D-glucose. The buffer temperature is maintained at 37° C., and the chamber is gassed with 5% CO2/95% O2 to maintain a pH of 7.4. Additionally, 5 μM cocaine and 1 μM (±)-propranolol may be added to block neuronal catecholoamine uptake and β-adrenoceptor activation by high noradrenaline concentration. Following equilibration, the vessels are challenged several times with 125 mM KCl and 10 μM noradrenaline. The vessels are then treated with 100 μM carbachol; vessels with a relaxation response of at least 50% indicate a functionally intact epithelium.

### Example 5

### Assesment of Intracellular Free Calcium Concentrations in Cultured Rat Aortic Smooth Muscle Cells

This example describes the assessment of intracellular free calcium concentrations in cultured rat aortic smooth muscle cells, as described in, for example, Bischoff, A. et al. (2000) Br. J. Pharmacol. 130:1871-1877. Vascular smooth muscle cells are prepared from rat thoracic aorta according to Rosskoph et al. (1995) Cell Physiol. Biochem. 5:276-285). Briefly, freshly prepared aortae are incubated for 30 minutes at room temperature with 125 U/ml collagenase I in Hank's balanced salt solution (HBSS) of the following composition: 118 mM NaCl, 5 mM KCl, 1 mM CaCl₂, 1 mM MgCl₂, 5 mM D-glucose, and 15 mM HEPES pH 7.4. Thereafter, remaining connective tissue and endothelium are removed, the aortae are cut into small pieces and incubated for 4-6 hours at 37° C. in DMEM/F12 medium with 100 U/ml penicillin, 100 μg/ml streptomycin, and 250 ng/ml amphotericin B. Treatment with collagenase (125 U/ml) and elastase (0.5 mg/ml) in HBSS without Ca²⁺ and Mg²⁺ follow for 2 hours at 37° C. . The reaction is stopped by addition of DMEM/F12 medium containing 20% fetal calf serum and penicillin, streptomycin, and amphotericin B, and the cells are plated onto 60-mm cell culture plates. The cells are used between passage 3 and 6. The Ca²⁺ concentration measurements are performed as described in Meyer zu Heringdorf et al. (1996) Naunyn-Schmiedeberg 's Arch. Pharmacol. 354:397-403. Briefly, the cells are loaded with 1 μM fura2/AM for 1 hour at room temperature in HBSS, washed with HBSS, and used for fluorescence measurements within the next hour. Ca²⁺ concentrations are measured in a continuously stirred cell suspension at room temperature in a Hitachi F2000 spectrofluorometer as described in Meyer zu Heringdorf et al. (1996) supra.

### Example 6

### Calcium Transport Assay

This example describes the assessment of calcium transport by HEAT molecules in cultured COS-1 cells, as described in, for example, Maruyama, K. and MacLennan, D. H. (1988) Proc. Natl. Acad. Sci. USA 85:3314-3318.

Cell Culture and DNA Transfection

COS-1 or HEK-293 cells are maintained in Dulbecco's modified Eagle's medium (DMEM) with 0.1 mM α-MEM nonessential amino acids, 4 mM L-glutamine, 100 units of pennicillin per ml, 100 μg of streptomycin per ml, and 10% fetal calf serum under 5% CO₂/95% air at 37° C. Transfection of HEAT-containing DNA is carried out by the DEAE dextran-chloroquine shock method (Sompayrac, L. M. and Danna, K. J. (1981) Proc. Natl. Acad. Sci. USA 78:7575-7579; Gorman, C. (1985) in DNA Cloning: A Practical Approach, ed. Gover, D. M. (IRL, Washington, D.C.), Vol. 2, pp. 143-190) with 25 μg of cesium chloride gradient-purified DNA and 1.5 mg of DEAE dextran per 10 cm Petri dish. Cells are then incubated for 3 hours at 37° C. in 6 ml of DMEM containing 300 μg of chloroquine, washed, and cultured in DMEM for 48 or 72 hours. Control cells are treated in the same way with vector DNA or with no added DNA.

Isolation of Microsomal Fraction

For isolation of a microsomal fraction (Resh, M. D. and Erikson, R. L. (1985) J. Cell Biol. 100:409-417; Yamada, S. and Ikemoto, N. (1980) J. Biol. Chem. 255:3108-3119), cells from five 10 cm Petri dishes are washed twice with 5 ml of a solution of 0.137 M NaCl/2.7 mM KCl/8 mM Na₂HPO₄/1.5 mM KH₂PO₄ (PBS), harvested in a solution of 5 mM EDTA in PBS and washed with 5 ml of PBS. The cells are swollen at 0° C. for 10 minutes in 2 ml of a hypotonic solution of 10 mM Tris-HCl, pH 7.5/0.5 mM MgCl₂, and then phenylmethylsolfonyl fluoride and Trasylol are added to 0.1 mM and 100 units/ml, respectively. The cells are homogenized with 30 strokes in a glass Dounce homogenizer, and the homogenate is diluted with an equal volume of a solution of 0.5 M sucrose/6 mM 2-mercaptoenthanol, 40 μM CaCl₂/300 mM KCl/10 mM Tris-HCl, pH 7.5. The suspension is centrifuged at 10,000×g for 20 minutes to pellet nuclei and mitochondria. The supernatant is brought to a concentration of 0.6 M KCl by the addition of 0.9 ml of a 2.5 M solution. The suspension is centrifuged at 100,000×g for 60 minutes to sediment the microsomal fraction. The pellet is suspended in a solution containing 0.25 M sucrose, 0.15 M KCl, 3 mM 2-mercaptoethanol, 20 μM CaCl₂, 10 mM Tris-HCl (pH 7.5), and centrifuged again at 100,000×g for 60 minutes. The final pellet, containing approximately 100 μg of protein, is suspended in the same solution at a protein concentration of 1 mg/ml.

Ca²⁺ Transport Assay

Ca²⁺ transport activity is assayed in a reaction mixture containing 20 μM Mops-KOH (pH 6.8), 100 mM KCl, 5 mM CaCl₂, 5 mM ATP, 0.45 mM CaCl₂ (containing ⁴⁵Ca at a specific activity of 10⁶ cpM/μmol), 0.5 mM EGTA, and 5 mM potassium oxalate. The uptake reaction is initiated by the addition of 10 μg of microsomal protein to 1 ml of reaction mixture at room temperature. At different time points, 0.15 ml samples are filtered through a 0.3 μm Millipore filter and washed with 10 ml of 0.15 M KCl. Radioactivity on the filter is measured by liquid scintillation counting. For the measurement of Ca²⁺ ion dependency, free Ca²⁺ concentration is calculated by the computer program of Fabiato and Fabiato ((1979) J. Physiol. (London) 75:463-505). For the measurement of ATP dependency, an ATP regenerating system consisting of 2.5 mM phosphoenolpyruvate and 50 μg of pyruvate kinase per ml is used.

Measurement of Phosphorylated HEAT Intermediate

Microsomal protein (5 μg) is added to 0.1 ml of a solution of 20 mM Mops, pH 6.8/100 mM KCl/5 mM MgCl₂/0.5 mM EGTA in the presence or absence of 0.5 mM CaCl_{2.} The reaction, at ice temperature, is started by the addition of 5 μM ATP (10⁶ cpm/nmol) and stopped after 5 seconds by the addition of 0.6 ml of a mixture of 5% trichloroacetic acid and 5 mM potassium phosphate. Incorporation of ³²P is determined either by collecting the protein on a filter for scintillation counting or by separating the protein in acidic NaDodSO₄/polyacrylamide gels for autoradiography Sarkadi, B. et al. (1986) J. Biol. Chem. 261:9552-9557).

### Example 7

### Analysis of HEAT-3 Activity

The full-length HEAT-3 was inserted into the multiple cloning site in the pCDNA3 vector. The DNA for the clone was amplified and transfected into HEK-293 cells using calcium phosphate precipitation. After 72 hours, the cells were harvested, microsomal fractions isolated, and ⁴⁵Ca-uptake measured as a function of calcium concentration using a filter assay.

Two different HEAT-3 fusion proteins were generated. One HEAT-3 fusion protein was created by inserting the 3×Flag epitope at the 3′ end of the HEAT-3 gene. Another HEAT-3 fusion protein was created by inserting the green fluorescent protein (GFP) at the 3′ end of the HEAT-3 gene. Fluorescence of this protein could be observed with the naked eye and by confocal microscopy. Measurement of expression using Western blotting with an anti-GFP antibody showed that HEAT-3 is well expressed in the microsomal fraction.

Confocal microscopy showed that the expression pattern of HEAT-3 is similar to SERCA1, indicating that HEAT-3 is targeted to and localized in the endoplasmic reticulum of HEK-293 cells.

Ca²⁺ uptake experiments (as described in Example 6) were performed using both the Flag and GFP fusion proteins. An increase in Ca²⁺ uptake of HEAT-3 was shown over GFP vector alone. Five independent experiments were performed to confirm the increase of calcium uptake with HEAT-3 as compared to vector alone. The Flag fusion protein showed an increase of calcium uptake as compared to Flag vector alone. In these experiments, the Vmax for HEAT-3 was lower (about 20×) than the Vmax for SERCA1 under the same conditions. The KCa for HEAT-3 was about 6.00 pCa units, as compared with about 6.38 pCa units for SERCA1. In the presence of ATP, there is 2-3 fold more calcium uptake compared to uptake in the absence of ATP, indicating that the calcium uptake by HEAT-3 is ATP dependent.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. An isolated nucleic acid molecule selected from the group consisting of:
(a) a nucleic acid molecule comprising the nucleotide sequence set forth in SEQ ID NO:1, 5, or 8; and
(b) a nucleic acid molecule comprising the nucleotide sequence set forth in SEQ ID NO:3, 7, or 10.

2. An isolated nucleic acid molecule which encodes a polypeptide comprising the amino acid sequence set forth in SEQ ID NO:2, 6, or 9.

3. An isolated nucleic acid molecule comprising the nucleotide sequence contained in the plasmid deposited with ATCC® as Accession Number ______, ______, or ______.

4. An isolated nucleic acid molecule which encodes a naturally-occurring allelic variant of a polypeptide comprising the amino acid sequence set forth in SEQ ID NO:2, 6, or 9.

5. An isolated nucleic acid molecule selected from the group consisting of:
(a) a nucleic acid molecule comprising a nucleotide sequence which is at least 60% identical to the nucleotide sequence of SEQ ID NO:1, 3, 5, 7, 8, or 10, or a complement thereof;
(b) a nucleic acid molecule comprising a fragment of at least 30 nucleotides of a nucleic acid comprising the nucleotide sequence of SEQ ID NO:1, 3, 5, 7, 8, or 10, or a complement thereof;
(c) a nucleic acid molecule which encodes a polypeptide comprising an amino acid sequence at least about 60% identical to the amino acid sequence of SEQ ID NO:2, 6, or 9; and
(d) a nucleic acid molecule which encodes a fragment of a polypeptide comprising the amino acid sequence of SEQ ID NO:2, 6, or 9, wherein the fragment comprises at least 10 contiguous amino acid residues of the amino acid sequence of SEQ ID NO:2, 6, or 9.

6. An isolated nucleic acid molecule which hybridizes to a complement of the nucleic acid molecule of any one of claims 1, 2, 3, 4, or 5 under stringent conditions.

7. An isolated nucleic acid molecule comprising a nucleotide sequence which is complementary to the nucleotide sequence of the nucleic acid molecule of any one of claims 1, 2, 3, 4, or 5.

8. An isolated nucleic acid molecule comprising the nucleic acid molecule of any one of claims 1, 2, 3, 4, or 5, and a nucleotide sequence encoding a heterologous polypeptide.

9. A vector comprising the nucleic acid molecule of any one of claims 1, 2, 3, 4, or 5.

10. The vector of claim 9, which is an expression vector.

11. A host cell transfected with the expression vector of claim 10.

12. A method of producing a polypeptide comprising culturing the host cell of claim 1 in an appropriate culture medium to, thereby, produce the polypeptide.

13. An isolated polypeptide selected from the group consisting of:
a) a fragment of a polypeptide comprising the amino acid sequence of SEQ ID NO:2, 6, or 9, wherein the fragment comprises at least 10 contiguous amino acids of SEQ ID NO:2, 6, or 9;
b) a naturally occurring allelic variant of a polypeptide comprising the amino acid sequence of SEQ ID NO:2, 6, or 9, wherein the polypeptide is encoded by a nucleic acid molecule which hybridizes to complement of a nucleic acid molecule consisting of SEQ ID NO:1, 3, 5, 7, 8, or 10 under stringent conditions;
c) a polypeptide which is encoded by a nucleic acid molecule comprising a nucleotide sequence which is at least 60% identical to a nucleic acid comprising the nucleotide sequence of SEQ ID NO:1, 3, 5, 7, 8, or 10; and
d) a polypeptide comprising an amino acid sequence which is at least 60% identical to the amino acid sequence of SEQ ID NO:2, 6, or 9.

14. The isolated polypeptide of claim 13 comprising the amino acid sequence of SEQ ID NO:2, 6, or 9.

15. The polypeptide of claim 13, further comprising heterologous amino acid sequences.

16. An antibody which selectively binds to a polypeptide of claim 13.

17. A method for detecting the presence of a polypeptide of claim 13 in a sample comprising:
a) contacting the sample with a compound which selectively binds to the polypeptide; and
b) determining whether the compound binds to the polypeptide in the sample to thereby detect the presence of a polypeptide of claim 13 in the sample.

18. The method of claim 17, wherein the compound which binds to the polypeptide is an antibody.

19. A kit comprising a compound which selectively binds to a polypeptide of claim 13 and instructions for use.

20. A method for detecting the presence of a nucleic acid molecule of any one of claims 1, 2, 3, 4, or 5 in a sample comprising:
a) contacting the sample with a nucleic acid probe or primer which selectively hybridizes to the nucleic acid molecule: and
b) determining whether the nucleic acid probe or primer binds to a nucleic acid molecule in the sample to thereby detect the presence of a nucleic acid molecule of any one of claims 1, 2, 3, 4, or 5 in the sample.

21. The method of claim 20, wherein the sample comprises mRNA molecules and is contacted with a nucleic acid probe.

22. A kit comprising a compound which selectively hybridizes to a nucleic acid molecule of any one of claims 1, 2, 3, 4, or 5 and instructions for use.

23. A method for identifying a compound which binds to a polypeptide of claim 13 comprising:
a) contacting the polypeptide, or a cell expressing the polypeptide with a test compound; and
b) determining whether the polypeptide binds to the test compound.

24. The method of claim 23, wherein the binding of the test compound to the polypeptide is detected by a method selected from the group consisting of:
a) detection of binding by direct detection of test compound/polypeptide binding;
b) detection of binding using a competition binding assay; and
c) detection of binding using an assay for HEAT activity.

25. A method for modulating the activity of a polypeptide of claim 13 comprising contacting the polypeptide or a cell expressing the polypeptide with a compound which binds to the polypeptide in a sufficient concentration to modulate the activity of the polypeptide.

26. A method for identifying a compound which modulates the activity of a polypeptide of claim 13 comprising:
a) contacting a polypeptide of claim 13 with a test compound; and
b) determining the effect of the test compound on the activity of the polypeptide to thereby identify a compound which modulates the activity of the polypeptide.

27. A method of identifying a subject having a cardiovascular disorder, or at risk for developing a cardiovascular disorder comprising:
a) contacting a sample obtained from said subject comprising nucleic acid molecules with a hybridization probe comprising at least 25 contiguous nucleotides of SEQ ID NO:1, 5, or 8; and
b) detecting the presence of a nucleic acid molecule in said sample that hybridizes to said probe, thereby identifying a subject having a cardiovascular disorder.

28. A method of identifying a subject having a cardiovascular disorder, or at risk for developing a cardiovascular disorder comprising:
a) contacting a sample obtained from said subject comprising nucleic acid molecules with a first and a second amplification primer, said first primer comprising at least 25 contiguous nucleotides of SEQ ID NO:1, 5, or 8 and said second primer comprising at least 25 contiguous nucleotides from the complement of SEQ ID NO:1, 5, or 8;
b) incubating said sample under conditions that allow nucleic acid amplification; and
c) detecting the presence of a nucleic acid molecule in said sample that is amplified, thereby identifying a subject having a cardiovascular disorder, or at risk for developing a cardiovascular disorder.

29. A method of identifying a subject having a cardiovascular disorder, or at risk for developing a cardiovascular comprising:
a) contacting a sample obtained from said subject comprising polypeptides with a HEAT binding substance; and
b) detecting the presence of a polypeptide in said sample that binds to said HEAT binding substance, thereby identifying a subject having a cardiovascular disorder, or at risk for developing a cardiovascular disorder.

30. A method for identifying a compound capable of treating a cardiovascular disorder characterized by aberrant HEAT nucleic acid expression or HEAT polypeptide activity comprising assaying the ability of the compound to modulate HEAT nucleic acid expression or HEAT polypeptide activity, thereby identifying a compound capable of treating a cardiovascular disorder characterized by aberrant HEAT nucleic acid expression or HEAT polypeptide activity.

31. A method for treating a subject having a cardiovascular disorder characterized by aberrant HEAT polypeptide activity or aberrant HEAT nucleic acid expression comprising administering to the subject a HEAT modulator, thereby treating said subject having a cardiovascular disorder.


SEQUENCE LISTING (52 sequences)

SEQ ID NO: 1 - DNA, 4055 bp, Homo sapiens
  CDS (210)..(3749)
tactatagggagtcgacccacgcgtccggccgcgccgagcctggtggcccaggtgccccgcccgcgtcagccctgctccagccccgcgctagcccagcgcccctcgccccgggccgtccggaccgcgcccccgcccagggccttgcgcacgccggggcccaggccgagggccgcagcgccggggccggcgatgagcgcgaggagccggcatgagcgcagacagcagccctctcgtgggcagcacgcccaccggttatgggaccctgacgatagggacatcaatagatcccctcagctcctcagtttcatccgtgaggctcagcggctactgtggcagtccatggagggtcatcggctatcacgtcgtggtctggatgatggctgggatccctttgctgctcttccgttggaagcccctgtggggggtgcggctgcggctccggccctgcaacctggcccacgccgaaacactcgttatcgaaataagagacaaagaggatagttcctggcagctcttcactgtccaggtgcagactgaggccatcggcgagggcagcctggagccgtccccacagtcccaggcagaggatggccggagccaggcggcagttggggcggtaccagagggtgcctggaaggatacggcccagctccacaagagcgaggaggcggtgagtgtcggacagaagcgggtgctgcggtattacctcttccagggccagcgctatatctggatcgagacccagcaagccttctaccaggtcagcctcctggaccatggccgctcttgtgacgacgtccaccgctcccgccatggcctcagcctccaggaccaaatggtgaggaaggccatttacggccccaacgtgatcagcataccggtcaagtcctacccccagctgctggtggacgaggcactgaacccctactatgggttccaggccttcagcatcgcgctgtggctggctgaccactactactggtacgccctgtgcatcttcctcatttcctccatctccatctgcctgtcgctgtacaagaccagaaagcaaagccagactctaagggacatggtcaagttgtccatgcgggtgtgcgtgtgccggccagggggagaggaagagtgggtggactccagtgagctagtgcccggagactgcctggtgctgccccaggagggtgggctgatgccctgtgatgccgccctggtggccggcgagtgcatggtgaatgagagctctctgacaggagagagcattccagtgctgaagacggcactgccggaggggctggggccctactgtgcagagacacaccggcggcacacactcttctgcgggaccctcatcttgcaggcccgggcctatgtgggaccgcacgtcctggcagtggtgacccgcacagggttctgcacggcaaaagggggcctggtgagctccatcttgcacccccggcccatcaacttcaagttctataaacacagcatgaagtttgtggctgccctctctgtcctggctctcctcggcaccatctacagcatcttcatcctctaccgaaaccgggtgcctctgaatgagattgtaatccgggctctcgacctggtgaccgtggtggtgccacctgccctgcctgctgccatgactgtgtgcacgctctacgcccagagccgactgcggagacagggcattttctgcatccacccactgcgcatcaacctggggggcaagctgcagctggtgtgtttcgacaagacgggcaccctcactgaggacggcttagacgtgatgggggtggtgcccctgaaggggcaggcattcctgcccctggtcccagagcctcgccgcctgcctgtggggcccctgctccgagcactggccacctgccatgccctcagccggctccaggacacccccgtgggcgaccccatggacttgaagatggtggagtctactggctgggtcctggaggaagagccggctgcagactcagcatttgggacccaggtcttggcagtgatgagacctccactttgggagccccagctgcaggcaatggaggagcccccggtgccagtcagcgtcctccaccgcttccccttctcttcggctctgcagcgcatgagtgtggtggtggcgtggccaggggccactcagcccgaggcctacgtcaaaggctccccggagctggtggcagggctctgcaaccccgagacagtgcccaccgacttcgcccagatgctgcagagctatacagctgctggctaccgtgtcgtggccctggccagcaagccactgcccactgtgcccagcctggaggcagcccagcaactgacgagggacactgtggaaggagacctgagcctcctggggctgctggtcatgaggaacctactgaagccgcagacaacgccagttatccaggctctgcgaaggacccgcatccgcgccgtcatggtgacaggggacaacctgcagacagcggtgactgtggcccggggctgtggcatggtggccccccaggagcatctgatcatcgtccacgccacccaccctgagcggggtcagcctgcctctctcgagttcctgccgatggagtcccccacagccgtgaatggcgttaaggatcctgaccaggctgcaagctacaccgtggagccagacccccgatccaggcacctggccctcagcgggcccacctttggtatcattgtgaagcacttccccaagctgctgcccaaggtcctggtccagggcactgtctttgcccgcatggcccctgagcagaagacagagctggtgtgcgagctacagaagcttcagtactgcgtgggcatgtgcggagacggtgccaatgactgtggggccctgaaggcggctgatgtcggcatctcgctgtcccaggcagaagcctcagtggtctcacccttcacctcgagcatggccagtattgagtgcgtgcccatggtcatcagggaggggcgctgttcccttgacacttcgttcagcgtcttcaagtacatggctctgtacagcctgacccagttcatctccgtcctgatcctctacacgatcaacaccaacctgggtgacctgcagttcctggccatcgacctggtcatcaccaccacagtggcagtgctcatgagccgcacggggccagcgctggtcctgggacgggtacggccaccgggggcgctgctcagcgtgcccgtgctcagcagcctgctgctgcagatggtcctggtgaccggcgtgcagctagggggctacttcctgaccctggcccagccatggttcgtgcctctgaacaggacagtggccgcaccagacaacctgcccaactacgagaacaccgtggtcttctctctgtccagcttccagtacctcatcctggctgcagccgtgtccaagggggcgcccttccgccggccgctctacaccaatgtgcccttcctggtggccctggcgctcctgagctccgtcctggtgggccttgtcctggtccccggcctcctgcaggggccgctggcgctgaggaacatcactgacaccggcttcaagctgctgctgctgggtctggtcaccctcaacttcgtgggggccttcatgctggagagcgtgctagaccagtgcctccccgcctgcctgcgccgcctccggcccaagcgggcctccaagaagcgcttcaagcagctggaacgagagctggccgagcagccctggccaccgctgcccgccggccccctgaggtagtgcaggcccacgggcaccccagacactggaactccctgcctctgagccaccaactggacccctctccagcaacaccaccgccaccacctcccacatccctgaggttggcgactgtctacactcctcccccgagaccacccccaccctggggaagcgttgactactgtcccctaccttggaccatcccgcgtaggggtggcagcccccagctcccctcagtgctgctgtcagtgtagcaaataaagtcatgatattttcctggcaaaaaaaaaaaaaaaaaaaaaaaaaaaaaaaaaaaaaaaa

SEQ ID NO: 2 - protein, 1180 aa, Homo sapiens
MSADSSPLVGSTPTGYGTLTIGTSIDPLSSSVSSVRLSGYCGSPWRVIGYHVVVWMMAGIPLLLFRWKPLWGVRLRLRPCNLAHAETLVIEIRDKEDSSWQLFTVQVQTEAIGEGSLEPSPQSQAEDGRSQAAVGAVPEGAWKDTAQLHKSEEAVSVGQKRVLRYYLFQGQRYIWIETQQAFYQVSLLDHGRSCDDVHRSRHGLSLQDQMVRKAIYGPNVISIPVKSYPQLLVDEALNPYYGFQAFSIALWLADHYYWYALCIFLISSISICLSLYKTRKQSQTLRDMVKLSMRVCVCRPGGEEEWVDSSELVPGDCLVLPQEGGLMPCDAALVAGECMVNESSLTGESIPVLKTALPEGLGPYCAETHRRHTLFCGTLILQARAYVGPHVLAVVTRTGFCTAKGGLVSSILHPRPINFKFYKHSMKFVAALSVLALLGTIYSIFILYRNRVPLNEIVIRALDLVTVVVPPALPAAMTVCTLYAQSRLRRQGIFCIHPLRINLGGKLQLVCFDKTGTLTEDGLDVMGVVPLKGQAFLPLVPEPRRLPVGPLLRALATCHALSRLQDTPVGDPMDLKMVESTGWVLEEEPAADSAFGTQVLAVMRPPLWEPQLQAMEEPPVPVSVLHRFPFSSALQRMSVVVAWPGATQPEAYVKGSPELVAGLCNPETVPTDFAQMLQSYTAAGYRVVALASKPLPTVPSLEAAQQLTRDTVEGDLSLLGLLVMRNLLKPQTTPVIQALRRTRIRAVMVTGDNLQTAVTVARGCGMVAPQEHLIIVHATHPERGQPASLEFLPMESPTAVNGVKDPDQAASYTVEPDPRSRHLALSGPTFGIIVKHFPKLLPKVLVQGTVFARMAPEQKTELVCELQKLQYCVGMCGDGANDCGALKAADVGISLSQAEASVVSPFTSSMASIECVPMVIREGRCSLDTSFSVFKYMALYSLTQFISVLILYTINTNLGDLQFLAIDLVITTTVAVLMSRTGPALVLGRVRPPGALLSVPVLSSLLLQMVLVTGVQLGGYFLTLAQPWFVPLNRTVAAPDNLPNYENTVVFSLSSFQYLILAAAVSKGAPFRRPLYTNVPFLVALALLSSVLVGLVLVPGLLQGPLALRNITDTGFKLLLLGLVTLNFVGAFMLESVLDQCLPACLRRLRPKRASKKRFKQLERELAEQPWPPLPAGPLR

SEQ ID NO: 3 - DNA, 3540 bp, Homo sapiens
atgagcgcagacagcagccctctcgtgggcagcacgcccaccggttatgggaccctgacgatagggacatcaatagatcccctcagctcctcagtttcatccgtgaggctcagcggctactgtggcagtccatggagggtcatcggctatcacgtcgtggtctggatgatggctgggatccctttgctgctcttccgttggaagcccctgtggggggtgcggctgcggctccggccctgcaacctggcccacgccgaaacactcgttatcgaaataagagacaaagaggatagttcctggcagctcttcactgtccaggtgcagactgaggccatcggcgagggcagcctggagccgtccccacagtcccaggcagaggatggccggagccaggcggcagttggggcggtaccagagggtgcctggaaggatacggcccagctccacaagagcgaggaggcggtgagtgtcggacagaagcgggtgctgcggtattacctcttccagggccagcgctatatctggatcgagacccagcaagccttctaccaggtcagcctcctggaccatggccgctcttgtgacgacgtccaccgctcccgccatggcctcagcctccaggaccaaatggtgaggaaggccatttacggccccaacgtgatcagcataccggtcaagtcctacccccagctgctggtggacgaggcactgaacccctactatgggttccaggccttcagcatcgcgctgtggctggctgaccactactactggtacgccctgtgcatcttcctcatttcctccatctccatctgcctgtcgctgtacaagaccagaaagcaaagccagactctaagggacatggtcaagttgtccatgcgggtgtgcgtgtgccggccagggggagaggaagagtgggtggactccagtgagctagtgcccggagactgcctggtgctgccccaggagggtgggctgatgccctgtgatgccgccctggtggccggcgagtgcatggtgaatgagagctctctgacaggagagagcattccagtgctgaagacggcactgccggaggggctggggccctactgtgcagagacacaccggcggcacacactcttctgcgggaccctcatcttgcaggcccgggcctatgtgggaccgcacgtcctggcagtggtgacccgcacagggttctgcacggcaaaagggggcctggtgagctccatcttgcacccccggcccatcaacttcaagttctataaacacagcatgaagtttgtggctgccctctctgtcctggctctcctcggcaccatctacagcatcttcatcctctaccgaaaccgggtgcctctgaatgagattgtaatccgggctctcgacctggtgaccgtggtggtgccacctgccctgcctgctgccatgactgtgtgcacgctctacgcccagagccgactgcggagacagggcattttctgcatccacccactgcgcatcaacctggggggcaagctgcagctggtgtgtttcgacaagacgggcaccctcactgaggacggcttagacgtgatgggggtggtgcccctgaaggggcaggcattcctgcccctggtcccagagcctcgccgcctgcctgtggggcccctgctccgagcactggccacctgccatgccctcagccggctccaggacacccccgtgggcgaccccatggacttgaagatggtggagtctactggctgggtcctggaggaagagccggctgcagactcagcatttgggacccaggtcttggcagtgatgagacctccactttgggagccccagctgcaggcaatggaggagcccccggtgccagtcagcgtcctccaccgcttccccttctcttcggctctgcagcgcatgagtgtggtggtggcgtggccaggggccactcagcccgaggcctacgtcaaaggctccccggagctggtggcagggctctgcaaccccgagacagtgcccaccgacttcgcccagatgctgcagagctatacagctgctggctaccgtgtcgtggccctggccagcaagccactgcccactgtgcccagcctggaggcagcccagcaactgacgagggacactgtggaaggagacctgagcctcctggggctgctggtcatgaggaacctactgaagccgcagacaacgccagttatccaggctctgcgaaggacccgcatccgcgccgtcatggtgacaggggacaacctgcagacagcggtgactgtggcccggggctgtggcatggtggccccccaggagcatctgatcatcgtccacgccacccaccctgagcggggtcagcctgcctctctcgagttcctgccgatggagtcccccacagccgtgaatggcgttaaggatcctgaccaggctgcaagctacaccgtggagccagacccccgatccaggcacctggccctcagcgggcccacctttggtatcattgtgaagcacttccccaagctgctgcccaaggtcctggtccagggcactgtctttgcccgcatggcccctgagcagaagacagagctggtgtgcgagctacagaagcttcagtactgcgtgggcatgtgcggagacggtgccaatgactgtggggccctgaaggcggctgatgtcggcatctcgctgtcccaggcagaagcctcagtggtctcacccttcacctcgagcatggccagtattgagtgcgtgcccatggtcatcagggaggggcgctgttcccttgacacttcgttcagcgtcttcaagtacatggctctgtacagcctgacccagttcatctccgtcctgatcctctacacgatcaacaccaacctgggtgacctgcagttcctggccatcgacctggtcatcaccaccacagtggcagtgctcatgagccgcacggggccagcgctggtcctgggacgggtacggccaccgggggcgctgctcagcgtgcccgtgctcagcagcctgctgctgcagatggtcctggtgaccggcgtgcagctagggggctacttcctgaccctggcccagccatggttcgtgcctctgaacaggacagtggccgcaccagacaacctgcccaactacgagaacaccgtggtcttctctctgtccagcttccagtacctcatcctggctgcagccgtgtccaagggggcgcccttccgccggccgctctacaccaatgtgcccttcctggtggccctggcgctcctgagctccgtcctggtgggccttgtcctggtccccggcctcctgcaggggccgctggcgctgaggaacatcactgacaccggcttcaagctgctgctgctgggtctggtcaccctcaacttcgtgggggccttcatgctggagagcgtgctagaccagtgcctccccgcctgcctgcgccgcctccggcccaagcgggcctccaagaagcgcttcaagcagctggaacgagagctggccgagcagccctggccaccgctgcccgccggccccctgagg

SEQ ID NO: 4 - protein, 1187 aa, Caenorhabditis elegans
MTLESGDHTLTLFAYRTGPFRTILFYALTVLTLGIFRLILHWKQKWDVKMRMVPCTFEAAEYIYIIDNHNVSELQPVLRKSNATIPTENGEMRKVPELRWFVYRKLEYVWIDDLNSDESVDEISDNDNCWKTSFEIANRIPCRSLLAVSESNFGLTLSEISRRLEFYGRNEIVVQLRPILYLLVMEVITPFYVFQIFSVTVWYNDEYAYYASLIVILSLGSIVMDVYQIRTQEIRLRSMVHSTESVEVIREGTEMTIGSDQLVPGDILLIPPHGCLMQCDSVLMNGTVIVNESVLTGESVPITKVALTDETNDSVFNIEKNSKNVLFCGTQVLQTRFYRGKKVKAIVLRTAYSTLKGQLVRSIMYPKPVDFRFTKDLFKFILFLACISGCGFIYTIIVMIMRGNTLRRIIVRSLDIITITVPPALPAAMSVGIINAQLRLKKKEIFCISPSTINTCGAINVVCFDKTGTLTEDGLDFHVVRPVMSAVNQEIQKVKLEKSNRTEFMGEMTELTSRNGLPFDGDLVKAIATCHSLTRINGVLHGDPLDLILFQKTGWTMEEGIEGDIEEETQRFDNVQPSIIKPTDDKSAEYSVIRQFTFSSSLQRMSVIVFDPREDRPDNMMLYSKGSPEMILSLCDPNTVPEDYLLQVNSYAQHGFRLIAVARRPLDLNFNKASKVKRDAVECDLEMLGLIVMENRVKPVTLGVINQLNRANIRTVMVTGDNLLTGLSVARECGIIRPSKRAFLVEHVPGELDEYGRTKIFVKQSVSSSDEVIEDDASVSISMCSSTWKGSSEGDGFSPTNTEVETPNPVTADSLGHLIASSYHLAISGPTFAVIVHEYPELVDQLCSVCDVFARMAPDQKQSLVEQLQQIDYTVAMCGDGANDCAALKAAHAGISLSDAEASIAAPFTSKVPDIRCVPTVISEGRAALVTSFGIFKYMAGYSLTQFVTVMHLYWISNILTDGQFMYIDMFLITMFALLFGNTPAFYRLAHTPPPTRLLSIASMTSVVGQLIIIGVVQFIVFFSTSQQPWFTPYQPPVDDEVEDKRSMQGTALFCVSMFQYIILALVYSKGPPFRGNLWSNKPIYKKKRSIEAIIDYVPTTNSDHIRRPSINGVTSSRTESTLLSAEGQQLHMTTSKNGKGGENPHSSALFERLISRIGGEPTWLTNPIPPHSLSEPEEPEKLERTY

SEQ ID NO: 5 - DNA, 7249 bp, Homo sapiens
  CDS (225)..(3995)
cacgcgtccgggctgggctgaggcgaggcggcggcggcgacagcggcggccgggtcccccgcggcccctggggctggtccggccgcgagggaggccgcggaggaggcggcgcggcggcggccagtgagcggccccgatctgacagacatccctgaatcttggtgtttggacataggagtgatcttccattgtttgaagcactggacctttaatccactgtaggtatggacagggaagaaaggaagaccatcaatcagggtcaagaagatgaaatggagatttatggttacaatttgagtcgctggaagcttgccatagtttctttaggagtgatttgctctgatgggtttctcctcctcctcctctattggatgcctgagtggcgggtgaaagcgacctgtgtcagagctgcaattaaagactgtgaagtagtgctgctgaggactactgatgaattcaaaatgtggttttgtgcaaaaattcgcgttctttctttggaaacttacccagtttcaagtccaaaatctatgtctaataagctttcaaatggccatgcagtttgtttaattgagaatcccactgaagaaaataggcacaggatcagtaaatattcacagactgaatcacaacagattcgttatttcacccaccatagtgtaaaatatttctggaatgataccattcacaattttgatttcttaaagggactggatgaaggtgtttcttgtacgtcaatttatgaaaagcatagtgcaggactgacaaaggggatgcatgcctacagaaaactgctttatggagtaaatgaaattgctgtaaaagtgccttctgtttttaagcttctaattaaagaggttctcaacccattttacattttccagctgttcagtgttatactgtggagcactgatgaatactattactatgctctagctattgtggttatgtccatagtatcaatcgtaagctcactatattccattagaaagcaatatgttatgttgcatgacatggtggcaactcatagtaccgtaagagtttcagtttgtagagtaaatgaagaaatagaagaaatcttttctaccgaccttgtgccaggagatgtcatggtcattccattaaatgggacaataatgccttgtgatgctgtgcttattaatggtacctgcattgtaaacgaaagcatgttaacaggagaaagtgttccagtgacaaagactaatttgccaaatccttcagtggatgtgaaaggaataggagatgaattatataatccagaaacacataaacgacatactttgttttgtgggacaactgttattcagactcgtttctacactggagaactcgtcaaagccatagttgttagaacaggatttagtacttccaaaggacagcttgttcgttccatattgtatcccaaaccaactgattttaaactctacagagatgcctacttgtttctactatgtcttgtggcagttgctggcattgggtttatctacactattattaatagcattttaaatgaggtacaagttggggtcataattatcgagtctcttgatattatcacaattactgtgccccctgcacttcctgctgcaatgactgctggtattgtgtatgctcagagaagactgaaaaaaatcggtattttctgtatcagtcctcaaagaataaatatttgtggacagctcaatcttgtttgctttgacaagactggaactctaactgaagatggtttagatctttgggggattcaacgagtggaaaatgcacgatttctttcaccagaagaaaatgtgtgcaatgagatgttggtaaaatcccagtttgttgcttgtatggctacttgtcattcacttacaaaaattgaaggagtgctctctggtgatccacttgatctgaaaatgtttgaggctattggatggattctggaagaagcaactgaagaagaaacagcacttcataatcgaattatgcccacagtggttcgtcctcccaaacaactgcttcctgaatctacccctgcaggaaaccaagaaatggagctgtttgaacttccagctacttatgagataggaattgttcgccagttcccattttcttctgctttgcaacgtatgagtgtggttgccagggtgctgggggataggaaaatggacgcctacatgaaaggagcgcccgaggccattgccggtctctgtaaacctgaaacagttcctgtcgattttcaaaacgttttggaagacttcactaaacagggcttccgtgtgattgctcttgcacacagaaaattggagtcaaaactgacatggcataaagtacagaatattagcagagatgcaattgagaacaacatggattttatgggattaattataatgcagaacaaattaaagcaagaaacccctgcagtacttgaagatttgcataaagccaacattcgcaccgtcatggtcacaggtgacagtatgttgactgctgtctctgtggccagagattgtggaatgattctacctcaggataaagtgattattgctgaagcattacctccaaaggatgggaaagttgccaaaataaattggcattatgcagactccctcacgcagtgcagtcatccatcagcaattgacccagaggctattccggttaaattggtccatgatagcttagaggatcttcaaatgactcgttatcattttgcaatgaatggaaaatcattctcagtgatactggagcattttcaagaccttgttcctaagttgatgttgcatggcaccgtgtttgcccgtatggcacctgatcagaagacacagttgatagaagcattgcaaaatgttgattattttgttgggatgtgtggtgatggcgcaaatgattgtggtgctttgaagagggcacacggaggcatttccttatcggagctcgaagcttcagtggcatctccctttacctctaagactcctagtatttcctgtgtgccaaaccttatcagggaaggccgtgctgctttaataacttccttctgtgtgtttaaattcatggcattgtacagcattatccagtacttcagtgttactctgctgtattctatcttaagtaacctaggagacttccagtttctcttcattgatctggcaatcattttggtagtggtatttacaatgagtttaaatcctgcctggaaagaacttgtggcacaaagaccaccttcgggtcttatatctggggcccttctcttctccgttttgtctcagattatcatctgcattggatttcaatctttgggttttttttgggtcaaacagcaaccttggtatgaagtgtggcatccaaaatcagatgcttgtaatacaacaggaagcgggttttggaattcttcacacgtagacaatgaaaccgaacttgatgaacataatatacaaaattatgaaaataccacagtgttttttatttccagttttcagtacctcatagtggcaattgccttttcaaaaggaaaacccttcaggcaaccttgctacaaaaattatttttttgttttttctgtgatttttttatatatttttatattattcatcatgttgtatccagttgcctctgttgaccaggttcttcagatagtgtgtgtaccatatcagtggcgtgtaactatgctcatcattgttcttgtcaatgcctttgtgtctatcacagtggagaacttcttccttgacatggtcctttggaaagttgtgttcaaccgagacaaacaaggagagtatcggttcagcaccacacagccaccgcaggagtcagtggatcggtggggaaaatgctgcttaccctgggccctgggctgtagaaagaagacaccaaaggcaaagtacatgtatctggcgcaggagctcttggttgatccagaatggccaccaaaacctcagacaaccacagaagctaaagctttagttaaggagaatggatcatgtcaaatcatcaccataacatagcagtgaatcagtctcagtggtattgctgatagcagtattcaggaatatgtgattttaggagtttctgatcctgtgtgtcagaatggcactagttcagtttatgtcccttctgatatagtagcttatttgacagctttgctcttccttaaaataaaaacagaaaaatatatcgtcctaacagttaaattaacaatcaatccataaagtcctatatcttcattcagcaacccaaatattacatacatttccagaattttcttgattgttactttcagtgatattctttatattgggtacaggagaagtttggtgtttggtaggtttttcaacattagtttttgagactagtttacctcttcacatttatgctcacaaccctcttgttagaaaagtctgtgtttatatacaggctgtaagtttgtgattgataaaaagaagatgagtgttaattagcctccagtgaaaatatactgaaagcctgttttcatttgattccaatgtttcttccaaagaattctgtataaacatatgccaattccctatgatggtctagagttaggaatgagtgtttatggtgttgcttatagaacaactcaggtaatctccatttctggttttatattttctgtacaaactgcctgggttttatttttctaatcagcaaggtgcttcactgccttcttgagacgcctctcaaagctcttaaatggctcctgtgctatgtgtggtgttggcagtctaatttgcttctgttaaatgttgtagaacctttttcactaggaaataagattcatttctttcggcagtagatgtagattcatcttttaacgtttcttcaaatttgtttctgtcaggctttgtgttattttaaatggttttttaaaattttcttctatgttttcaattacctaaagacataggataatagttttttttaagttagaattttacctcataaaattttttgaggtttgatgtatgtctctgtcttatcaataatgaggcttaaaaaatactggatttgaatggctgccgttttttcaaagcaatatgaatttgatgagtttgttttatgccattaggtggcgccagaggtcagaacatgtctattttgaattggatcgttacaaatgagcatatttgatgcggaaatttctgggagaaaaaaaattgaggaaataaagttaaaaaattgacattcattgagccaaaagagatgtggagaaacatttttcacctttctgtttggcctgattaacatttaaattcttgccaaaattaaggtaacttttaaaaaacaccttttataggtggatccagcagtctggcaacgcccacagttaccacaacacagaaaactgatcgtgctataaaatggacgctaaactatgaaaacagtgtgacattgttctctgttcttccagagccagtaacatgcttgctcgtgctttctacttctagctgatcattcttttcccaacatatatttacaaattaccaaattttacctagaattttaggaccaaatggttctcactctttatgctgcaaagacctggatgatgtttggtaactatagaaaaatagaaattacactcaggatcactgttactgctattgccactgatgattcctgcaaaaatataatcgaagttttccatcaaatgtataatatgctattaatacacattagatgataacagttgttccatgaatgattctatgaagctatgcatcttagacctcttgagctgtgaattagcactattttctatagttacttattctctggatcattttataatttccatattaatttcaaatatgctcgtgttattcttcagtgatttccacaattgtgcattttattctttggtttaagtactgaagcatataatgaaagtaattgctaagtagcagcttaaaaattcaattatccgattgtatttaacatctttaagagcatgatcataaagagctatttttgacaccccccccccacttttttaacatttagagttagtaagggttttatatctcttctgtccatattgttttcaaaggaatgaggtgtttaggtggctggaaaagcatttgtaggaagttagatttgaatatagacaaggtgggttattcacgttgagaatgttatttgaagaatgcctgtgaagccaggtgtgggttctactcagtgccatagatagactgagtcttctctcgtaggttaccattacatagtaattttgattctgaattacacattaaattatttgagtttatacagacctaaattttaaaatctgtacatatattattttgatgtattaagatgaatattgctgatttaaattttatttatgcacatacttaaaggacagaaatgtctgggaaagtaattgttaaataatgatatgtaactttttaactttttaaataaataacaagatttttaatgtgtgtctccctcagggttgtttaaagttttttttctccctcaagtataaatagtggtaactatatgttttgtatcttctagcaccaactgctgtaaagcaatgctgcaaataatgcttgaatacaagtggctaagccaacaacagaataaatacttttatagtagttttataatcctgaaattcgaaagctttcccaattgcacttgcatctaaacaaaactgttgcagtttttactctatttattttgttccccatgtttatgaaagtcctgcacagtttcaaaggcatggtaaataatatatcaatgtttatgtagtctgttacagaaacagctatagataacattatccagtgaagagcaaaattcaagctttagaaaatattcatgcatgcaattttgacatatctaaaaataggtttttgtatatttatggtgggaggtggttgggaacttttaacaaaatggggtgttaatttttgtacagtctgtgggcatttacacatttttaatgtattaaaatttggtaattatgtgtacattaaattaataaaagttacttctagttatgatttgtgaattccctaagaccttggatttttttaagtaactttatatcagaaatgatactgcatctttatatttttaaaattgtattgctgctcaagaatggtaccctcttgtcaaaaaggcatacattcataattgtacattcagcattgtaaataatcttatgaaaccttttttgattgaagctattcaaaataaaaattttaatgaatgaaaaaaaaaaaaaaaa

SEQ ID NO: 6 - protein, 1256 aa, Homo sapiens
MDREERKTINQGQEDEMEIYGYNLSRWKLAIVSLGVICSDGFLLLLLYWMPEWRVKATCVRAAIKDCEVVLLRTTDEFKMWFCAKIRVLSLETYPVSSPKSMSNKLSNGHAVCLIENPTEENRHRISKYSQTESQQIRYFTHHSVKYFWNDTIHNFDFLKGLDEGVSCTSIYEKHSAGLTKGMHAYRKLLYGVNEIAVKVPSVFKLLIKEVLNPFYIFQLFSVILWSTDEYYYYALAIVVMSIVSIVSSLYSIRKQYVMLHDMVATHSTVRVSVCRVNEEIEEIFSTDLVPGDVMVIPLNGTIMPCDAVLINGTCIVNESMLTGESVPVTKTNLPNPSVDVKGIGDELYNPETHKRHTLFCGTTVIQTRFYTGELVKAIVVRTGFSTSKGQLVRSILYPKPTDFKLYRDAYLFLLCLVAVAGIGFIYTIINSILNEVQVGVIIIESLDIITITVPPALPAAMTAGIVYAQRRLKKIGIFCISPQRINICGQLNLVCFDKTGTLTEDGLDLWGIQRVENARFLSPEENVCNEMLVKSQFVACMATCHSLTKIEGVLSGDPLDLKMFEAIGWILEEATEEETALHNRIMPTVVRPPKQLLPESTPAGNQEMELFELPATYEIGIVRQFPFSSALQRMSVVARVLGDRKMDAYMKGAPEAIAGLCKPETVPVDFQNVLEDFTKQGFRVIALAHRKLESKLTWHKVQNISRDAIENNMDFMGLIIMQNKLKQETPAVLEDLHKANIRTVMVTGDSMLTAVSVARDCGMILPQDKVIIAEALPPKDGKVAKINWHYADSLTQCSHPSAIDPEAIPVKLVHDSLEDLQMTRYHFAMNGKSFSVILEHFQDLVPKLMLHGTVFARMAPDQKTQLIEALQNVDYFVGMCGDGANDCGALKRAHGGISLSELEASVASPFTSKTPSISCVPNLIREGRAALITSFCVFKFMALYSIIQYFSVTLLYSILSNLGDFQFLFIDLAIILVVVFTMSLNPAWKELVAQRPPSGLISGALLFSVLSQIIICIGFQSLGFFWVKQQPWYEVWHPKSDACNTTGSGFWNSSHVDNETELDEHNIQNYENTTVFFISSFQYLIVAIAFSKGKPFRQPCYKNYFFVFSVIFLYIFILFIMLYPVASVDQVLQIVCVPYQWRVTMLIIVLVNAFVSITVENFFLDMVLWKVVFNRDKQGEYRFSTTQPPQESVDRWGKCCLPWALGCRKKTPKAKYMYLAQELLVDPEWPPKPQTTTEAKALVKENGSCQIITIT

SEQ ID NO: 7 - DNA, 3768 bp, Homo sapiens
atggacagggaagaaaggaagaccatcaatcagggtcaagaagatgaaatggagatttatggttacaatttgagtcgctggaagcttgccatagtttctttaggagtgatttgctctgatgggtttctcctcctcctcctctattggatgcctgagtggcgggtgaaagcgacctgtgtcagagctgcaattaaagactgtgaagtagtgctgctgaggactactgatgaattcaaaatgtggttttgtgcaaaaattcgcgttctttctttggaaacttacccagtttcaagtccaaaatctatgtctaataagctttcaaatggccatgcagtttgtttaattgagaatcccactgaagaaaataggcacaggatcagtaaatattcacagactgaatcacaacagattcgttatttcacccaccatagtgtaaaatatttctggaatgataccattcacaattttgatttcttaaagggactggatgaaggtgtttcttgtacgtcaatttatgaaaagcatagtgcaggactgacaaaggggatgcatgcctacagaaaactgctttatggagtaaatgaaattgctgtaaaagtgccttctgtttttaagcttctaattaaagaggttctcaacccattttacattttccagctgttcagtgttatactgtggagcactgatgaatactattactatgctctagctattgtggttatgtccatagtatcaatcgtaagctcactatattccattagaaagcaatatgttatgttgcatgacatggtggcaactcatagtaccgtaagagtttcagtttgtagagtaaatgaagaaatagaagaaatcttttctaccgaccttgtgccaggagatgtcatggtcattccattaaatgggacaataatgccttgtgatgctgtgcttattaatggtacctgcattgtaaacgaaagcatgttaacaggagaaagtgttccagtgacaaagactaatttgccaaatccttcagtggatgtgaaaggaataggagatgaattatataatccagaaacacataaacgacatactttgttttgtgggacaactgttattcagactcgtttctacactggagaactcgtcaaagccatagttgttagaacaggatttagtacttccaaaggacagcttgttcgttccatattgtatcccaaaccaactgattttaaactctacagagatgcctacttgtttctactatgtcttgtggcagttgctggcattgggtttatctacactattattaatagcattttaaatgaggtacaagttggggtcataattatcgagtctcttgatattatcacaattactgtgccccctgcacttcctgctgcaatgactgctggtattgtgtatgctcagagaagactgaaaaaaatcggtattttctgtatcagtcctcaaagaataaatatttgtggacagctcaatcttgtttgctttgacaagactggaactctaactgaagatggtttagatctttgggggattcaacgagtggaaaatgcacgatttctttcaccagaagaaaatgtgtgcaatgagatgttggtaaaatcccagtttgttgcttgtatggctacttgtcattcacttacaaaaattgaaggagtgctctctggtgatccacttgatctgaaaatgtttgaggctattggatggattctggaagaagcaactgaagaagaaacagcacttcataatcgaattatgcccacagtggttcgtcctcccaaacaactgcttcctgaatctacccctgcaggaaaccaagaaatggagctgtttgaacttccagctacttatgagataggaattgttcgccagttcccattttcttctgctttgcaacgtatgagtgtggttgccagggtgctgggggataggaaaatggacgcctacatgaaaggagcgcccgaggccattgccggtctctgtaaacctgaaacagttcctgtcgattttcaaaacgttttggaagacttcactaaacagggcttccgtgtgattgctcttgcacacagaaaattggagtcaaaactgacatggcataaagtacagaatattagcagagatgcaattgagaacaacatggattttatgggattaattataatgcagaacaaattaaagcaagaaacccctgcagtacttgaagatttgcataaagccaacattcgcaccgtcatggtcacaggtgacagtatgttgactgctgtctctgtggccagagattgtggaatgattctacctcaggataaagtgattattgctgaagcattacctccaaaggatgggaaagttgccaaaataaattggcattatgcagactccctcacgcagtgcagtcatccatcagcaattgacccagaggctattccggttaaattggtccatgatagcttagaggatcttcaaatgactcgttatcattttgcaatgaatggaaaatcattctcagtgatactggagcattttcaagaccttgttcctaagttgatgttgcatggcaccgtgtttgcccgtatggcacctgatcagaagacacagttgatagaagcattgcaaaatgttgattattttgttgggatgtgtggtgatggcgcaaatgattgtggtgctttgaagagggcacacggaggcatttccttatcggagctcgaagcttcagtggcatctccctttacctctaagactcctagtatttcctgtgtgccaaaccttatcagggaaggccgtgctgctttaataacttccttctgtgtgtttaaattcatggcattgtacagcattatccagtacttcagtgttactctgctgtattctatcttaagtaacctaggagacttccagtttctcttcattgatctggcaatcattttggtagtggtatttacaatgagtttaaatcctgcctggaaagaacttgtggcacaaagaccaccttcgggtcttatatctggggcccttctcttctccgttttgtctcagattatcatctgcattggatttcaatctttgggttttttttgggtcaaacagcaaccttggtatgaagtgtggcatccaaaatcagatgcttgtaatacaacaggaagcgggttttggaattcttcacacgtagacaatgaaaccgaacttgatgaacataatatacaaaattatgaaaataccacagtgttttttatttccagttttcagtacctcatagtggcaattgccttttcaaaaggaaaacccttcaggcaaccttgctacaaaaattatttttttgttttttctgtgatttttttatatatttttatattattcatcatgttgtatccagttgcctctgttgaccaggttcttcagatagtgtgtgtaccatatcagtggcgtgtaactatgctcatcattgttcttgtcaatgcctttgtgtctatcacagtggagaacttcttccttgacatggtcctttggaaagttgtgttcaaccgagacaaacaaggagagtatcggttcagcaccacacagccaccgcaggagtcagtggatcggtggggaaaatgctgcttaccctgggccctgggctgtagaaagaagacaccaaaggcaaagtacatgtatctggcgcaggagctcttggttgatccagaatggccaccaaaacctcagacaaccacagaagctaaagctttagttaaggagaatggatcatgtcaaatcatcaccataaca

SEQ ID NO: 8 - DNA, 3919 bp, Homo sapiens
  CDS (68)..(3679)
ttaccggaagtaaaacttcggaagtgaggcgttcctctgcccggaagtgagcgccgcgctaggaaagatggcggcagcggcggcggtgggcaacgcggtgccctgcggggcccggccttgcggggtccggcctgacgggcagcccaagcccgggccgcagccgcgcgcgctccttgccgccgggccggcgctcatagcgaacggtgacgagctggtggctgccgtgtggccgtaccggcggttggcgctgttgcggcgcctcacggtgctgccattcgccgggctgctttacccggcctggttgggtgccgcagccgctggctgctggggctggggcagcagttgggtgcagatccccgaagctgcgctgctcgtgcttgccaccatctgcctcgcgcacgcgctcactgtcctctcggggcattggtctgtgcacgcgcattgcgcgctcacctgcaccccggagtacgaccccagcaaagcgacctttgtgaaggtggcgccaacccccaacaatggctccacggagctcgtggccctgcaccgcaatgagggcgaagacgggcttgaggtgctgtccttcgaattccagaagatcaagtattcctacgatgccctggagaagaagcagtttctccccgtggcctttcctgtgggaaacgccttctcatactatcagagcaacagaggcttccaggaagactcagagatccgagcagctgagaagaaatttgggagcaacaaggccgagatggtggtgcctgacttctcggagcttttcaaggagagagccacagcccccttctttgtatttcaggtgttctgtgtggggctctggtgcctggatgagtactggtactacagcgtctttacgctatccatgctggtggcgttcgaggcctcgctggtgcagcagcagatgcggaacatgtcggagatccggaagatgggcaacaagccccacatgatccaggtctaccgaagccgcaagtggaggcccattgccagtgatgagatcgtaccaggggacatcgtctccatcggccgctccccacaggagaacctggtgccatgtgacgtgcttctgctgcgaggccgctgcatcgtagacgaggccatgctcacgggggagtccgtgccacagatgaaggagcccatcgaagacctcagcccagaccgggtgctggacctccaggctgattcccggctgcacgtcatcttcgggggcaccaaggtggtgcagcacatccccccacagaaagccaccacgggcctgaagccggttgacagcgggtgcgtggcctacgtcctgcggaccggattcaacacatctcagggcaagctgctgcgcaccatcctcttcggggtcaagagggtgactgcgaacaacctggagaccttcatcttcatcctcttcctcctggtgtttgccatcgctgcagctgcctatgtatggattgaaggtaccaaggaccccagccggaaccgctacaagctgtttctggagtgcaccctgatcctcacctcggtcgtgcctcctgagctgcccatcgagctgtccctggccgtcaacacctccctcatcgccctggccaagctctacatgtactgcacagagcccttccggatcccctttgctggcaaggtcgaggtgtgctgctttgacaagacggggacgttgaccagtgacagcctggtggtgcgcggtgtggccgggctgagagacgggaaggaggtgaccccagtgtccagcatccctgtagaaacacaccgggccctggcctcgtgccactcgctcatgcagctggacgacggcaccctcgtgggtgaccctctagagaaggccatgctgacggccgtggactggacgctgaccaaagatgagaaagtattcccccgaagtattaaaactcaggggctgaaaattcaccagcgctttcattttgccagtgccctgaagcgaatgtccgtgcttgcctcgtatgagaagctgggctccaccgacctctgctacatcgcggccgtgaagggggcccccgaaactctgcactccatgttctcccagtgcccgcccgactaccaccacatccacaccgagatctcccgggaaggagcccgcgtcctggcgctggggtacaaggagctgggacacctcactcaccagcaggcccgggaggtcaagcgggaggccctggagtgcagcctcaagttcgtcggcttcattgtggtctcctgcccgctcaaggctgactccaaggccgtgatccgggagatccagaatgcgtcccaccgggtggtcatgatcacgggagacaacccgctcactgcatgccacgtggcccaggagctgcacttcattgaaaaggcccacacgctgatcctgcagcctccctccgagaaaggccggcagtgcgagtggcgctccattgacggcagcatcgtgctgcccctggcccggggctccccaaaggcactggccctggagtacgcactgtgcctcacaggcgacggcttggcccacctgcaggccaccgacccccagcagctgctccgcctcatcccccatgtgcaggtgttcgcccgtgtggctcccaagcagaaggagtttgtcatcaccagcctgaaggagctgggctacgtgaccctcatgtgtggggatggcaccaacgacgtgggcgccctgaagcatgctgacgtgggtgtggcgctcttggccaatgcccctgagcgggttgtcgagcggcgacggcggccccgggacagcccaaccctgagcaacagtggcatcagagccacctccaggacagccaagcagcggtcggggctccctccctccgaggagcagccaacctcccagagggaccgcctgagccaggtgctgcgagacctcgaggacgagagtacgcccattgtgaaactgggggatgccagcatcgcagcacccttcacctccaagctctcatccatccagtgcatctgccacgtgatcaagcagggccgctgcacgctggtgaccacgctacagatgttcaagatcctggcgctcaatgccctcatcctggcctacagccagagcgtcctctacctggagggagtcaagttcagtgacttccaggccaccctacaggggctgctgctggccggctgcttcctcttcatctcccgttccaagcccctcaagaccctctcccgagaacggcccctgcccaacatcttcaacctgtacaccatcctcaccgtcatgctccagttctttgtgcacttcctgagccttgtctacctgtaccgtgaggcccaggcccggagccccgagaagcaggagcagttcgtggacttgtacaaggagtttgagccaagcctggtcaacagcaccgtctacatcatggccatggccatgcagatggccaccttcgccatcaattacaaaggcccgcccttcatggagagcctgcccgagaacaagcccctggtgtggagtctggcagtttcactcctggccatcattggcctgctcctcggctcctcgcccgacttcaacagccagtttggcctcgtggacatccctgtggagttcaagctggtcattgcccaggtcctgctcctggacttctgcctggcgctcctggccgaccgcgtcctgcagttcttcctggggaccccgaagctgaaagtgccttcctgagatggcagtgctggtacccactgcccaccctggctgccgctgggcgggaaccccaacagggccccgggagggaaccctgcccccaaccccccacagcaaggctgtacagtctcgcccttggaagactgagctgggacccccacagccatccgctggcttggccagcagaaccagccccaagccagcacctttggtaaataaagcagcatctgagattttaaaaaaaaaaaaaaaaaa

SEQ ID NO: 9 - protein, 1204 aa, Homo sapiens
MAAAAAVGNAVPCGARPCGVRPDGQPKPGPQPRALLAAGPALIANGDELVAAVWPYRRLALLRRLTVLPFAGLLYPAWLGAAAAGCWGWGSSWVQIPEAALLVLATICLAHALTVLSGHWSVHAHCALTCTPEYDPSKATFVKVAPTPNNGSTELVALHRNEGEDGLEVLSFEFQKIKYSYDALEKKQFLPVAFPVGNAFSYYQSNRGFQEDSEIRAAEKKFGSNKAEMVVPDFSELFKERATAPFFVFQVFCVGLWCLDEYWYYSVFTLSMLVAFEASLVQQQMRNMSEIRKMGNKPHMIQVYRSRKWRPIASDEIVPGDIVSIGRSPQENLVPCDVLLLRGRCIVDEAMLTGESVPQMKEPIEDLSPDRVLDLQADSRLHVIFGGTKVVQHIPPQKATTGLKPVDSGCVAYVLRTGFNTSQGKLLRTILFGVKRVTANNLETFIFILFLLVFAIAAAAYVWIEGTKDPSRNRYKLFLECTLILTSVVPPELPIELSLAVNTSLIALAKLYMYCTEPFRIPFAGKVEVCCFDKTGTLTSDSLVVRGVAGLRDGKEVTPVSSIPVETHRALASCHSLMQLDDGTLVGDPLEKAMLTAVDWTLTKDEKVFPRSIKTQGLKIHQRFHFASALKRMSVLASYEKLGSTDLCYIAAVKGAPETLHSMFSQCPPDYHHIHTEISREGARVLALGYKELGHLTHQQAREVKREALECSLKFVGFIVVSCPLKADSKAVIREIQNASHRVVMITGDNPLTACHVAQELHFIEKAHTLILQPPSEKGRQCEWRSIDGSIVLPLARGSPKALALEYALCLTGDGLAHLQATDPQQLLRLIPHVQVFARVAPKQKEFVITSLKELGYVTLMCGDGTNDVGALKHADVGVALLANAPERVVERRRRPRDSPTLSNSGIRATSRTAKQRSGLPPSEEQPTSQRDRLSQVLRDLEDESTPIVKLGDASIAAPFTSKLSSIQCICHVIKQGRCTLVTTLQMFKILALNALILAYSQSVLYLEGVKFSDFQATLQGLLLAGCFLFISRSKPLKTLSRERPLPNIFNLYTILTVMLQFFVHFLSLVYLYREAQARSPEKQEQFVDLYKEFEPSLVNSTVYIMAMAMQMATFAINYKGPPFMESLPENKPLVWSLAVSLLAIIGLLLGSSPDFNSQFGLVDIPVEFKLVIAQVLLLDFCLALLADRVLQFFLGTPKLKVPS

SEQ ID NO: 10 - DNA, 3612 bp, Homo sapiens
atggcggcagcggcggcggtgggcaacgcggtgccctgcggggcccggccttgcggggtccggcctgacgggcagcccaagcccgggccgcagccgcgcgcgctccttgccgccgggccggcgctcatagcgaacggtgacgagctggtggctgccgtgtggccgtaccggcggttggcgctgttgcggcgcctcacggtgctgccattcgccgggctgctttacccggcctggttgggtgccgcagccgctggctgctggggctggggcagcagttgggtgcagatccccgaagctgcgctgctcgtgcttgccaccatctgcctcgcgcacgcgctcactgtcctctcggggcattggtctgtgcacgcgcattgcgcgctcacctgcaccccggagtacgaccccagcaaagcgacctttgtgaaggtggcgccaacccccaacaatggctccacggagctcgtggccctgcaccgcaatgagggcgaagacgggcttgaggtgctgtccttcgaattccagaagatcaagtattcctacgatgccctggagaagaagcagtttctccccgtggcctttcctgtgggaaacgccttctcatactatcagagcaacagaggcttccaggaagactcagagatccgagcagctgagaagaaatttgggagcaacaaggccgagatggtggtgcctgacttctcggagcttttcaaggagagagccacagcccccttctttgtatttcaggtgttctgtgtggggctctggtgcctggatgagtactggtactacagcgtctttacgctatccatgctggtggcgttcgaggcctcgctggtgcagcagcagatgcggaacatgtcggagatccggaagatgggcaacaagccccacatgatccaggtctaccgaagccgcaagtggaggcccattgccagtgatgagatcgtaccaggggacatcgtctccatcggccgctccccacaggagaacctggtgccatgtgacgtgcttctgctgcgaggccgctgcatcgtagacgaggccatgctcacgggggagtccgtgccacagatgaaggagcccatcgaagacctcagcccagaccgggtgctggacctccaggctgattcccggctgcacgtcatcttcgggggcaccaaggtggtgcagcacatccccccacagaaagccaccacgggcctgaagccggttgacagcgggtgcgtggcctacgtcctgcggaccggattcaacacatctcagggcaagctgctgcgcaccatcctcttcggggtcaagagggtgactgcgaacaacctggagaccttcatcttcatcctcttcctcctggtgtttgccatcgctgcagctgcctatgtatggattgaaggtaccaaggaccccagccggaaccgctacaagctgtttctggagtgcaccctgatcctcacctcggtcgtgcctcctgagctgcccatcgagctgtccctggccgtcaacacctccctcatcgccctggccaagctctacatgtactgcacagagcccttccggatcccctttgctggcaaggtcgaggtgtgctgctttgacaagacggggacgttgaccagtgacagcctggtggtgcgcggtgtggccgggctgagagacgggaaggaggtgaccccagtgtccagcatccctgtagaaacacaccgggccctggcctcgtgccactcgctcatgcagctggacgacggcaccctcgtgggtgaccctctagagaaggccatgctgacggccgtggactggacgctgaccaaagatgagaaagtattcccccgaagtattaaaactcaggggctgaaaattcaccagcgctttcattttgccagtgccctgaagcgaatgtccgtgcttgcctcgtatgagaagctgggctccaccgacctctgctacatcgcggccgtgaagggggcccccgaaactctgcactccatgttctcccagtgcccgcccgactaccaccacatccacaccgagatctcccgggaaggagcccgcgtcctggcgctggggtacaaggagctgggacacctcactcaccagcaggcccgggaggtcaagcgggaggccctggagtgcagcctcaagttcgtcggcttcattgtggtctcctgcccgctcaaggctgactccaaggccgtgatccgggagatccagaatgcgtcccaccgggtggtcatgatcacgggagacaacccgctcactgcatgccacgtggcccaggagctgcacttcattgaaaaggcccacacgctgatcctgcagcctccctccgagaaaggccggcagtgcgagtggcgctccattgacggcagcatcgtgctgcccctggcccggggctccccaaaggcactggccctggagtacgcactgtgcctcacaggcgacggcttggcccacctgcaggccaccgacccccagcagctgctccgcctcatcccccatgtgcaggtgttcgcccgtgtggctcccaagcagaaggagtttgtcatcaccagcctgaaggagctgggctacgtgaccctcatgtgtggggatggcaccaacgacgtgggcgccctgaagcatgctgacgtgggtgtggcgctcttggccaatgcccctgagcgggttgtcgagcggcgacggcggccccgggacagcccaaccctgagcaacagtggcatcagagccacctccaggacagccaagcagcggtcggggctccctccctccgaggagcagccaacctcccagagggaccgcctgagccaggtgctgcgagacctcgaggacgagagtacgcccattgtgaaactgggggatgccagcatcgcagcacccttcacctccaagctctcatccatccagtgcatctgccacgtgatcaagcagggccgctgcacgctggtgaccacgctacagatgttcaagatcctggcgctcaatgccctcatcctggcctacagccagagcgtcctctacctggagggagtcaagttcagtgacttccaggccaccctacaggggctgctgctggccggctgcttcctcttcatctcccgttccaagcccctcaagaccctctcccgagaacggcccctgcccaacatcttcaacctgtacaccatcctcaccgtcatgctccagttctttgtgcacttcctgagccttgtctacctgtaccgtgaggcccaggcccggagccccgagaagcaggagcagttcgtggacttgtacaaggagtttgagccaagcctggtcaacagcaccgtctacatcatggccatggccatgcagatggccaccttcgccatcaattacaaaggcccgcccttcatggagagcctgcccgagaacaagcccctggtgtggagtctggcagtttcactcctggccatcattggcctgctcctcggctcctcgcccgacttcaacagccagtttggcctcgtggacatccctgtggagttcaagctggtcattgcccaggtcctgctcctggacttctgcctggcgctcctggccgaccgcgtcctgcagttcttcctggggaccccgaagctgaaagtgccttcc

SEQ ID NO: 11 - protein, 7 aa, Artificial Sequence
  misc_feature 6: Xaa may be Leu, Ile, Val, or Met
DKTGTXX

SEQ ID NO: 12 - protein, 1157 aa, Caenorhabditis elegans
MGVDQLVETIIPYNLRSIATHLYVPPFTIITAIWTYVWLNIFGYEEYYELGMLGYAAIFVILALVLLFCHWMMPVRCFLMCSKQEDVRIASHVCVIPTQNNGWPELVKLMRTTRDKQTKLWFEFQRVHYTWDEESREFQTKTLDTAKPMVFFQKSHGFEVEEHVKDAKYLLGDNKTEMIVPQFLEMFIERATAPFFVFQVFCVGLWCLEDMWYYSLFTLFMLMTFEATLVKQQMKNMSEIRNMGNKTYMINVLRGKKWQKIKIEELVAGDIVSIGRGAEEECVPCDLLLLRGPCIVDESMLTGESVPQMKEPIEDVEKDKIFDIETDSRLHVIFGGTKIVQHTAPGKAAEGMVKSPDGNCICYVIRTGFNTSQGKLLRTIMFGVKKATANNLETFCFILFLLIFAIAAAAYLWIKGSVDETRSKYKLFLECTLILTSVIPPELPIELSLAVNSSLMALQKLGIFCTEPFRIPFAGKVDICCFDKTGTLTTDNLVVEGVALNNQKEGMIRNAEDLPHESLQVLASCHSLVRFEEDLVGDPLEKACLSWCGWNLTKGDAVMPPKTAAKGISGIKIFHRYHFSSAMKRMTVVAGYQSPGTSDTTFIVAVKGAPEVLRNMYADLPSDYDETYTRLTRQGSRVLAMGIRKLGETRVGELRDKKRENFENDLAFAGFVVISCPLKSDTKTMIREIMDSSHVVAMITGDNPLTACHVSKVLKFTKKSLPTLVLDEPADGVDWMWKSVDGTIELPLKPETKNKMERKAFFNSHEFCLTGSAFHHLVHNEHTFLRELILHVKVFARMAPKQKERIINELKSLGKVTLMCGDGTNDVGALKHANVGVALLTNPYDAEKAAEKEKEKKAKIEEARSLVRSGAQLPQRPGAPGAPPAANAARPRLDNLMKELEEEEKAQVIKLGDASIAAPFTSKYTSIASICHVIKQGRCTLVTTLQMFKILALNALVSAYSLSALYLDGVKFSDTQATIQGLLLAACFLFISKSKPLKTLSRQRPMANIFNAYTLLTVTLQFIVHFSCLLYIVGLAHEANTEKAPVDLEAKFTPNILNTTVYIISMALQVCTFAVNYRGRPFMESLFENKAMLYSIMFSGGAVFTLASGQATDLMIQFELVVLPEALRNALLMCVTADLVICYIIDRGLNFLLGDMF

SEQ ID NO: 13 - protein, 9 aa, Artificial Sequence
  misc_feature 1: Xaa may be Asp, Asn, or Ser
XXXXXXGEX

SEQ ID NO: 14 - protein, 10 aa, Artificial Sequence
  misc_feature 1: Xaa may be Leu, Ile or Val
XXXDKTGTXT

SEQ ID NO: 15 - protein, 11 aa, Artificial Sequence
  misc_feature 1: Xaa may be Thr, Ile, or Val
XGDGXNDXPXL

SEQ ID NO: 16 - protein, 7 aa, Artificial Sequence
  Description of Artificial Sequenceamino acid residues important for calcium transport
IPEGLPA

SEQ ID NO: 17 - protein, 28 aa, Homo sapiens
DLVTVVVPPALPAAMTVCTLYAQSRLRR

SEQ ID NO: 18 - protein, 28 aa, Homo sapiens
DIITITVPPALPAAMTAGIVYAQRRLKK

SEQ ID NO: 19 - protein, 28 aa, Homo sapiens
LILTSVVPPELPIELSLAVNTSLIALAK

SEQ ID NO: 20 - protein, 29 aa, Saccharomyces cerevisiae
DIITIVVPPALPATLTIGTNFALSRLKEK

SEQ ID NO: 21 - protein, 28 aa, Saccharomyces cerevisiae
LIITSVVPPELPMELTMAVNSSLAALAK

SEQ ID NO: 22 - protein, 28 aa, Schizosaccharomyces pombe
VLTILVPPALPATLSVGIANSIARLSRA

SEQ ID NO: 23 - protein, 29 aa, Caenorhabditis elegans
DLVTIVVPPALPAVMGIGIFYAQRRLRQK

SEQ ID NO: 24 - protein, 29 aa, Caenorhabditis elegans
DIITIVVPPALPAAMSVGIINANSRLKKK

SEQ ID NO: 25 - protein, 29 aa, Caenorhabditis elegans
DIITITVPPALPAAMSVGIINAQLRLKKK

SEQ ID NO: 26 - protein, 29 aa, Caenorhabditis elegans
LILTSVIPPELPIELSLAVNSSLMALQKL

SEQ ID NO: 27 - protein, 29 aa, Homo sapiens
ALAVAAIPEGLPAVITTCLALGTRRMAKK

SEQ ID NO: 28 - protein, 29 aa, Oryctolagus cuniculus
ALAVAAIPEGLPAVITTCLALGTRRMAKK

SEQ ID NO: 29 - protein, 29 aa, Gallus gallus
ALAVAAIPEGLPAVITTCLALGTRRMAKK

SEQ ID NO: 30 - protein, 29 aa, Felis catus
ALAVAAIPEGLPAVITTCLALGTRRMAKK

SEQ ID NO: 31 - protein, 29 aa, Procambarus clarkii
ALAVAAIPEGLPAVITTCLALGTRRMAKK

SEQ ID NO: 32 - protein, 29 aa, Homo sapiens
ALAVAAIPEGLPAVITTCLALGTRRMAKK

SEQ ID NO: 33 - protein, 29 aa, Homo sapiens
ALAVAAIPEGLPAVITTCLALGTRRMARK

SEQ ID NO: 34 - protein, 29 aa, Drosophila melanogaster
AVAVAAIPEGLPAVITTCLALGTRRMAKK

SEQ ID NO: 35 - protein, 29 aa, Saccharomyces cerevisiae
SLAVAAIPEGLPIIVTVTLALGVLRMAKR

SEQ ID NO: 36 - protein, 29 aa, Saccharomyces cerevisiae
TVIVVAVPEGLPLAVTLALAFATTRMTKD

SEQ ID NO: 37 - protein, 29 aa, Homo sapiens
TVLVVAVPEGLPLAVTISLAYSVKKMMKD

SEQ ID NO: 38 - protein, 29 aa, Homo sapiens
TVLVVAVPEGLPLAVTISLAYSVKKMMKD

SEQ ID NO: 39 - protein, 29 aa, Rattus norvegicus
TVLVVAVPEGLPLAVTISLAYSVKKMMKD

SEQ ID NO: 40 - protein, 29 aa, Homo sapiens
TVLVVAVPEGLPLAVTISLAYSVKKMMKD

SEQ ID NO: 41 - protein, 29 aa, Homo sapiens
TVLVVAVPEGLPLAVTISLAYSVKKMMKD

SEQ ID NO: 42 - protein, 29 aa, Saccharomyces cerevisiae
TVLIVSCPCVIGLAVPIVFVIASGVAAKR

SEQ ID NO: 43 - protein, 29 aa, Homo sapiens
TVLCIACPCSLGLATPTAVMVGTGVGAQN

SEQ ID NO: 44 - protein, 29 aa, Homo sapiens
TVLCIACPCSLGLATPTAVMVGTGVAAQN

SEQ ID NO: 45 - protein, 29 aa, Drosophila melanogaster
GIIVANVPEGLLATVTVCLTLTAKRMASK

SEQ ID NO: 46 - protein, 29 aa, Hydra vulgaris
GIIVANVPEGLLATVTVCLTLTAKKMAKK

SEQ ID NO: 47 - protein, 29 aa, Bufo marinus
GIIVANVPEGLLATVTVCLTLTAKRMARK

SEQ ID NO: 48 - protein, 29 aa, Homo sapiens
GIIVANVPEGLLATVTVCLTLTAKRMARK

SEQ ID NO: 49 - protein, 29 aa, Homo sapiens
GIIVANVPEGLLATVTVCLTLTAKRMARK

SEQ ID NO: 50 - protein, 29 aa, Homo sapiens
GIIVANVPEGLLATVTVCLTVTAKRMARK

SEQ ID NO: 51 - protein, 29 aa, Homo sapiens
ILFNNLIPISLLVTLEVVKFTQAYFINWD

SEQ ID NO: 52 - protein, 29 aa, Saccharomyces cerevisiae
ILFSNLVPISLFVTVELIKYYQAFMIGSD